(19) United States
(12) Patent Application Publication
(10) Pub. No.: US 2025/0011781 A1
(43) Pub. Date: Jan. 9, 2025

(54) METHODS AND COMPOSITIONS TARGETING NUCLEUS ACCUMBENS-ASSOCIATED PROTEIN-1 FOR TREATMENT OF AUTOIMMUNE DISORDERS AND CANCERS

(71) Applicants: THE TEXAS A&M UNIVERSITY SYSTEM, College Station, TX (US); UNIVERSITY OF KENTUCKY RESEARCH FOUNDATION, Lexington, KY (US)

(72) Inventors: Jianxun SONG, Bryan, TX (US); Jin-Ming YANG, Lexington, KY (US)

(21) Appl. No.: 18/712,539

(22) PCT Filed: Nov. 22, 2022
(86) PCT No.: PCT/US22/80367

§ 371 Date: May 22, 2024

Related U.S. Application Data
(60) Provisional application No. 63/282,030, filed on Nov. 22, 2021.

(51) Int. Cl. C12N 15/113; A61K 31/165; A61P 35/00; C07K 16/18; C12N 9/22; C12N 15/11

(52) U.S. Cl. CPC C12N 15/113; A61K 31/165; A61P 35/00; C07K 16/18; C12N 9/22; C12N 15/111; C12N 2310/20

(57) ABSTRACT

Provided herein are methods of for enhancing or inducing an anti-tumor response or treating an autoimmune disorder by administering a therapeutically effective amount of an inhibitor of NAC 1. Also, provided herein are methods of enhancing effectiveness of a vaccine in a subject by administering to the subject a therapeutically effective amount of an inhibitor of NAC 1. Inhibitors of NAC1 can include a chemical agent, such as a composition containing NIC3, or a biological agent that inhibit the function of NAC1 protein, such as an isolated antibody or its binding fragment thereof that binds to NAC1. Inhibitors of NAC 1 can include a biological agent that reduces the expression of NAC1 gene, such as a NAC 1-targeted siRNA administered as a nanoliposome or a CRISPR/Cas-based genome editing composition targeting the NAC1 Gene.

### CROSS-REFERENCE TO RELATED APPLICATIONS

This application claims the benefit of and priority to U.S. Provisional Application No. 63/282,030, filed Nov. 22, 2021, which is incorporated by reference herein in its entirety.

### GOVERNMENT SUPPORT

This invention was made with United States government support under grant no. R01CA221867, R01AI121180, and R21AI167793 awarded by the National Institutes of Health and LC210150 by the Department of Defense. The Government has certain rights in the invention.

### TECHNICAL FIELD

The present disclosure related to methods and compositions for combating disorders involving the immune system by targeting nucleus accumbens-associated protein-1 (NAC1). The disclosure also includes chemical and biological agents that target NAC1 and are administered to a patient as a therapeutic strategy for treatment of a variety of disorders, including autoimmune disorders, cancers, and infections.

### BACKGROUND

Several disorders involve the immune system. For example, aberrant autoimmunity results in over 80 different autoimmune diseases that are often debilitating and life-threatening, for which there is no cure at present. Autoimmune diseases such as Crohn's disease, type-1 diabetes mellitus, rheumatoid arthritis, and ulcerative colitis are believed to result from interaction between genetic and environmental factors and to be a consequence of compromised immune tolerance versus adaptive immune response. Immune tolerance prevents an immune response to a particular antigen or tissues that cause autoimmune disorders, and a range of immune cell types participate in the control of hyposensitivity of the adaptive immune system to the self-antigen or non-self-antigen. Among these immune cells, FoxP3⁺ regulatory T cells (Tregs), a distinct and dynamic subset of CD4⁺ T cells, are an essential contributor to the immune tolerance, maintenance of immune cell homeostasis and the balance of the immune system. Defects in Tregs occur in virtually all the autoimmune disorders. The stability of the suppressor Tregs is critical for their function but is reduced in most of the autoimmune disorders. Therefore, maintenance of the Treg stability is crucial for immunologic tolerance. Yet, how impaired balance between immune response and tolerance is triggered and the key molecular determinants that affect Treg stability remain elusive.

As another example, immunotherapy has shown significant potential as a powerful approach to treat cancers by harnessing the body's immune system and numerous studies have demonstrated promising results of cancer immunotherapy. For instance, immunotherapy based on adoptive cell transfer (ACT) of ex vivo activated and expanded tumor-infiltrating T lymphocytes (TILs), has shown favorable clinical outcomes in patients with metastatic melanoma, one of the most aggressive and fatal neoplasms responsible for over 80% of skin cancer-related deaths. Yet, despite enormous advances in cancer immunotherapy, its clinical efficacy and benefits remain less satisfactory due to a variety of factors that limit antitumor immunity, and among those factors, the immune-suppressive tumor microenvironment (TME) is a major hindrance to successful treatment of cancer including melanoma. To circumvent the immune-evasive TME and enhance the efficacy of immunotherapeutic intervention, novel and effective TME targets are a pressing need.

Memory T cells are formed by the host in the process of eliminating invading pathogens. Upon repeated infection by the same pathogen, these memory T cells are able to respond quickly to provide protective immunity. This form of immunologic memory is vital for raising an immune response against many infectious agents such as viruses and bacteria. Memory CD8⁺ T cells play a critical role during acute or chronic viral infection and improving and prolonging CD8⁺ T cell memory could help strengthen the protective efficacy of vaccine design strategies and boost immune responses. Additionally, it has been appreciated that the memory T-cell-based immunotherapy has better efficacy than the effector T-cell-based immunotherapy in cancer treatments. Hence, the strategy to improve CD8⁺ T cell memory formation may provide effective prevention of virus reinfection and improve the efficacy of T-cell-based immunotherapy.

### SUMMARY

Applicant has developed compositions and methods of targeting NAC1 as a main treatment or an adjuvant therapy for several disorders.

Embodiments include methods for enhancing or inducing an anti-tumor response in a subject by administering to the subject a therapeutically effective amount of an inhibitor of expression or activity of NAC1. The anti-tumor response can be an increase in CD8⁺ T cell-mediated anti-tumor immunity or a persistent anti-tumor T cell memory. In certain embodiments, the subject has been administered an adoptive cell transfer therapy, such as a chimeric antigen receptor T-cell therapy or a tumor-infiltrating lymphocyte therapy. Embodiments include compositions containing a NAC1-targeted siRNA as an inhibitor of NAC1. The NAC1-targeted siRNA can be administered as a nanoliposome. The inhibitor of NAC1 can be a CRISPR/Cas-based genome editing composition comprising one or more vectors encoding: (a) one or more guide RNAs (gRNAs) that are complementary to one or more target sequences in a NAC1 gene and (b) a nucleic acid sequence encoding a Clustered Regularly interspaced Short Palindromic Repeat (CRISPR)-associated endonuclease, whereby the one or more gRNAs hybridize to the NAC1 gene and the CRISPR-associated endonuclease cleaves the NAC1 gene. The NAC1 sequence can be deleted in the patient relative to a patient to whom the CRISPR/Cas-based genome editing composition is not administered. The NAC1 expression or activity can be reduced in the patient relative to a patient to whom the CRISPR/Cas-based genome editing composition is not administered. The inhibitor of NAC1 can be an isolated antibody or its binding fragment thereof that binds to NAC1. The inhibitor of NAC1 corresponds to Formula I:

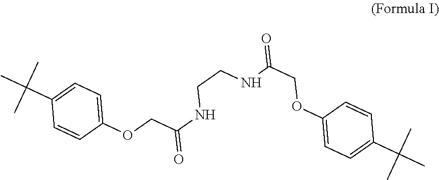 [SMILES: CC(C)(C)c1ccc(OCC(=O)NCCNC(=O)COc2ccc(C(C)(C)C)cc2)cc1]

Effective amounts of the inhibitor of NAC1 improve the tumor microenvironment through suppression of tumor cell metabolism and increases CD8⁺ T cell-mediated anti-tumor immunity. Methods also include administering to the patient an effective amount of NAC1-targeted siRNA nanoliposomes or CRISPR/Cas9 for enhancing or inducing an anti-tumor immune response including persistent anti-tumor T cell memory in the patient. The NAC1-targeted siRNA nanoliposomes or CRISPR/Cas9 compositions improve the tumor microenvironment through suppression of tumor cell metabolism and increase CD8⁺ T cell-mediated anti-tumor immunity. These chemical and biological agents that target NAC1 are also provided as adjuvants to T-cell-based immunotherapy.

Embodiments include methods of treating an autoimmune disorder by administering a therapeutically effective amount of an inhibitor of NAC1. In certain embodiments, the autoimmune disorder is autoimmune arthritis. In certain embodiments, the autoimmune disorder is autoimmune colitis.

Embodiments include methods of enhancing effectiveness of a vaccine in a subject by administering to the subject a therapeutically effective amount of an inhibitor of NAC1. The inhibitor of NAC1 can be administered before, after or concurrent with the vaccine. The vaccine can be a COVID-19 vaccine.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. To facilitate this description, like reference numerals designate like structural elements or procedures in a method. Embodiments are illustrated by way of example and not by way of limitation in the figures of the accompanying drawings.

FIGS. 1A-1O demonstrates the impact of the loss of NAC1 on overall T cell populations. FIGS. 1A and 1B are flow cytometry analyses of the CD4 and CD8 in the thymus of WT and NAC1^{-/-} mice. The double negative (DN) populations (shown as circles with arrows pointing outward) were analyzed for DN1 to DN4 stages based on CD44 and CD25. FIGS. 1C and 1D are flow cytometry analyses of for DN1 to DN4 stages based on CD44 and CD25. FIG. 1E is a graphical representation of the percentages of total thymocytes, CD4 or CD8 single positive (SP) and the percentages of DN2 or DN4 cells from WT and NAC1^{-/-} mice. FIGS. 1F and 1G are flow cytometry analyses of the CD4 and CD8 in the LNs and spleen of WT and NAC1^{-/-} mice. FIGS. 1H and 11 are flow cytometry analyses of the CD4 and CD8 cells gated on CD3⁺ populations in the LNs and spleen of WT and NAC1^{-/-} mice. FIGS. 1J and 1K are graphical representations of percentages of CD4 and CD8 T cells the LNs and spleen, respectively, of WT and NAC1^{-/-} mice. FIGS. 1L and 1M are flow cytometry analyses of the CD4 and FoxP3 cells in WT and NAC1^{-/-} mice, respectively. FIGS. 1N and 1O are graphical representations of percentages of Tregs cells in the LNs+spleen and thymus of WT and NAC1^{-/-} mice, respectively.

FIGS. 2A-2L depict the loss of NAC1 enhancing the induction of iT_{regs} and expression of CD36. The naive CD4⁺CD25⁻ T cells from the pooled spleen and LNs of WT or NAC1^{-/-} mice were induced to iT_{regs} in the presence of TGF-β. FIG. 2A is a photograph of an immunoblot showing expression of NAC1 in naive CD4 and iT_{regs} of WT T cells. FIG. 2B and FIG. 2C are flow cytometry analyses of expression of CD25 and FoxP3. FIG. 2D is a graphical representation of the percentages of CD25⁺FoxP3⁺ populations in WT and NAC1^{-/-} mice. FIG. 2E is a graphical representation of CD36 expression of untreated WT or NAC1^{-/-} Tregs when analyzed using flow cytometry. Tregs generated in vitro were treated with 10 mM lactic acid for various times and analyzed by flow cytometry for CD36 expression. FIG. 2F and FIG. 2G are flow cytometry analyses of expression of SSCA-A and CD36 in WT and NAC1^{-/-} mice, respectively, following 24 hours of lactic acid treatment. FIG. 2H and FIG. 2I are flow cytometry analyses of expression of SSCA-A and CD36 in WT and NAC1^{-/-} mice, respectively, following 48 hours of lactic acid treatment. FIG. 2J and FIG. 2K are flow cytometry analyses of expression of SSCA-A and CD36 in WT and NAC1^{-/-} mice, respectively, following 72 hours of lactic acid treatment. FIG. 2L is a graphical representation of the percentages of CD36⁺ Tregs following lactic acid treatment in WT and NAC1^{-/-} mice.

FIGS. 3A-3W depict the loss of NAC1 enhancing the functional activity of Tregs. Purified CD4⁺ Tregs from the pooled LNs and spleen of WT or NAC1^{-/-} mice were stimulated with anti-CD3 plus CD28 antibodies in the presence of rIL-2 for 6 hr. FIG. 3A is a graphical representation of CD4⁺ Tregs in WT or NAC1^{-/-} mice when analyzed using flow cytometry. FIGS. 3B and 3C are graphical representations of percentages and numbers, respectively, of cell recovery on various days of analyses. The numbers of T cells present on day 0 were assigned a value of 100%, and numbers surviving on various days were used to calculate the percentage recovery relative to day 0. FIGS. 3D and 3E are graphical representations of the Oxygen consumption rate (OCR, y axis) trace and tabulated data (FIG. 3E) of Tregs. FIGS. 3F and 3G are graphical representations of the glycolytic rate (glycoPER, y axis) trace and tabulated data (FIG. 3G) of Tregs. FIG. 3H, FIG. 3I, and FIG. 3J are flow cytometry analyses of expression of expression of TGF-β in control, WT, and NAC1^{-/-} Tregs, respectively. FIG. 3K is a graphical representation of number of FOXP3⁺ TGF-β⁺ cells in WT or NAC1^{-/-} Tregs when analyzed using flow cytometry. FIG. 3L, FIG. 3M, and FIG. 3N are flow cytometry analyses of expression of expression of IL-10 in control, WT, and NAC1^{-/-} Tregs, respectively. FIG. 3O is a graphical representation of number of FOXP3⁺IL-10⁺ cells in WT or NAC1^{-/-} Tregs when analyzed using flow cytometry. FIGS. 3P, 3Q, and 3R are graphical representations of results from in vitro suppressive assay. CD4⁺ CD25^{-/-} T effectors pre-labelled with CFSE were stimulated with anti-CD3 plus CD28 antibodies in the absence (FIG. 3P) or presence of WT (FIG. 3Q) or NAC1^{-/-} Tregs (FIG. 3R) (1:1) for three days. Cell proliferation was analyzed by flow cytometry, gating on CFSE⁺ population. FIG. 3S is a graphical representation of changes of body weight of Rag1^{-/-} host mice after adoptive cell transfer of naive CD4⁺ T effectors (Teffs) with or without Tregs from WT or NAC1^{-/-} mice. FIGS. 3T, 3U, 3V and 3W are representative photographic images of H&E-stained sections of cecum tissues collected from control mice (FIG. 3T) and the Rag1^{-/-} host mice after adoptive cell transfer of naive CD4⁺ T effectors (Teffs) (FIG. 3U) and with or without Tregs from WT or NAC1^{-/-} mice (FIG. 3V and FIG. 3W, respectively).

FIGS. 4A-4O demonstrate that NAC1^{-/-} mice are tolerant to induction of autoimmunity. WT or NAC1^{-/-} mice were challenged with either bovine type II collagen in complete Freund's adjuvant by one intradermal immunization at two sites in the base and slightly above of the tail on day 0, or by oral ingestion of 3% dextran sulfate sodium (DSS, MP Biomedicals) in drinking water for 5 days. FIGS. 4A-4D are photographic images of hematoxylin and safranin O staining of WT mice (FIGS. 4A and 4C) or NAC1^{-/-} mice (FIGS. 4B and 4D) to show the histology of the joints in an arthritic model. FIGS. 4E and 4F are graphical representations of arthritis incidence (FIG. 4E), and clinical score (FIG. 4F) as evaluated by examining the paws. FIGS. 4G-4J are photographic images of the histology of the colon in H₂O and DSS-challenged WT mice (FIGS. 4G and 4I) and H₂O and DSS-challenged NAC1^{-/-} mice (FIGS. 4H and 4J) to show a colitis model. The severity of colitis activity was graded on the designated dates. FIGS. 4K and 4L are graphical representations of animal body weight change (FIG. 4K) and survival (FIG. 4L) of the WT or NAC1^{-/-} mice. FIGS. 4M and 4N are photographic images of the WT or NAC1^{-/-} animals and their colon length, respectively. FIG. 4O is a graphical representation of the resultant IBD disease activity index.

FIGS. 5A-5H demonstrate the effects of NAC1 on proliferation of melanoma cells and development of melanoma. FIG. 5A is a graphical representation of the survival of melanoma patients with high expression (top 30%; red) and low expression of NAC1 (bottom 30%; blue), calculated with the ‘R2: Tumor Skin Cutaneous Melanoma-TCGA-470-rsem-tcgars’ dataset (http://r2.amc.nl) from the TCGA database. FIG. 5B is a graphical representation of the distribution of NAC1 expression in human skin cancer cell lines (melanoma, Merkel cell carcinoma, and skin squamous carcinoma), determined by the CCLE database (https://portals.broadinstitute.org/ccle). FIG. 5C is a photographical representation of the Western blot analysis of protein expression of NAC1 and β-actin in WT and NAC1-deficient (NAC1 KO) mouse B16-OVA melanoma cells. FIG. 5D is a photographical representation of the Western blot analysis of protein expression of NAC1 and j-actin in WT and NAC1 KO human A2058 melanoma cells. FIG. 5E and FIG. 5F are graphical representations of the cell division rates of WT and NAC1 KO mouse B16-OVA melanoma cells as analyzed by CFSE between day 1 and day 3. FIG. 5G and FIG. 5H are graphical representations of the cell division rates of WT and NAC1 KO human A2058 melanoma cells were analyzed by CFSE between day 1 and day 3. In these experiments, the tumor cells were labeled with CFSE dye (5 μM) prior to seeding onto the 48-well tissue culture plates. The CFSE intensities were analyzed by flow cytometer (FIG. 5E and FIG. 5G) and quantified (FIG. 5F and FIG. 511) by the mean fluorescence intensity (MFI) of CFSE. One representative of three identical experiments is shown.

FIGS. 6A-6J are graphical representations to demonstrate depleting NAC1 decreases the glycolytic rate of melanoma cells. FIG. 6A is a graphical representation of the time series of ECAR measurements in WT and NAC1 KO B16-OVA cells by the Seahorse Metabolic Analyzer. FIG. 6B is a graphical representation of the time series of ECAR measurements in WT and NAC1 KO A2058 cells by the Seahorse Metabolic Analyzer. Data are represented as mean±SEM; n=6 wells per condition from two independent experiments. FIG. 6C is a graphical representation of the basal glycolysis rates of WT and NAC1 KO B16-OVA cells. FIG. 6D is a graphical representation of the maximum glycolysis rates of WT and NAC1 KO B16-OVA cells. FIG. 6E is a graphical representation of the basal glycolysis rates of WT and NAC1 KO A2058 cells. FIG. 6F is a graphical representation of the maximum glycolysis rates of WT and NAC1 KO A2058 cells. In FIGS. 6C-6F, six wells per condition from two independent experiments were used and a one-way ANOVA with multiple comparisons correction was performed. FIG. 6G is a graphical representation of the quantification of relative glucose consumption in WT and NAC1 KO B16− OVA cells. FIG. 6H is a graphical representation of quantification of lactase production in the supernatant of WT and NAC1 KO B16-OVA cells. FIG. 6I is a graphical representation of the quantification of relative glucose consumption in WT and NAC1 KO A2058 cells. FIG. 6J is a graphical representation of the quantification of lactase production in the supernatant of WT and NAC1 KO A2058 cells.

FIGS. 7A-7Y demonstrate that depletion of tumorous NAC1 strengthens the cytotoxicity of CD8⁺ T cells. For FIG. 7A, WT or NAC1 KO B16-OVA cells (targets) were co-cultured with OT-I CD8⁺ T cells (effectors) (ratio: 1:5 and 1:10) for 6 hours. FIG. 7A is a graphical representation of the quantification of percent cytotoxicity when calculated as (1−(R5/R0))×100, where R5=(target cells as % of total of 6 hours)/(effector cells as % of total of 6 hours), R0=(target cells as % of total of 0 h)/(effector cells as % of total at 0 h). For FIG. 7B, WT or NAC1 KO A2058 cells were co-cultured with anti-tyrosinase TCR transduced human CD8⁺ T cells (effectors) (ratio: 1:5, 1:10, 1:20) for 6 hours. FIG. 7B is a graphical representation of the quantification of percent cytotoxicity was calculated as (1−(R5/R0))×100, where R5=(target cells as % of total at 6 hours)/(effector cells as % of total of 6 hours), R0=(target cells as % of total of 0 hour)/(effector cells as % of total of 0 hour). FIGS. 7C-7H are representative contour plots of Annexin V and Live-Dead expression after incubation with WT conditional medium (CM) or NAC1 KO B16-OVA CM for 12 hours (FIG. 7C and FIG. 7F), for 24 hours (FIG. 7D and FIG. 7G), and for 48 hours (FIG. 7E and FIG. 7H). FIG. 7I is a graphical representation of the frequencies of the indicated Annexin V⁺ and Live-Dead⁺ expressing populations after incubation with WT conditional medium (CM) or NAC1 KO B16-OVA CM for 12, 24, and 48 hours. Apoptotic rates were assessed by flow cytometry. One representative experiment out of three is shown. FIGS. 7J-70 are representative contour plots of Annexin V and Live-Dead expression on the CM-treated human CD8⁺ T cells after incubation with WT CM or NAC1 KO A2058 CM for 12 hours (FIG. 7J and FIG. 7M), for 24 hours (FIG. 7K and FIG. 7N), and for 48 hours (FIG. 7L and FIG. 7O). FIG. 7P is a graphical representation of the frequencies of the indicated Annexin V⁺ and Live-Dead⁺ expressing populations after incubation with WT CM or NAC1 KO A2058 CM for 12, 24, and 48 hours, respectively. Apoptotic rates were assessed by flow cytometry. One representative experiment out of three is shown. FIGS. 7Q-7V are histograms showing expression of the indicated cytokines after incubation with WT CM and NAC1 KO A2058 CM for 6 hours IL-2 (FIG. 7Q and FIG. 7T), IFN-γ (FIG. 7R and FIG. 7U), and Granzyme B (FIG. 7S and FIG. 7V), respectively. FIGS. 7Q-7V are histograms showing expression of the indicated cytokines after incubation with WT CM or NAC1 KO B16-OVA CM for 6 hours IL-2 (FIG. 7W), IFN-γ (FIG. 7X), and Granzyme B (FIG. 7Y), respectively. One representative of three identical experiments is shown.

FIGS. 8A-8J demonstrate NAC1 promotes the expression of LDHA in tumor cells. FIG. 8A is a heatmap of normalized RNA-seq reads (Z score), top upregulated glycolysis-associated genes in the TCGA SKCM database. FIG. 8B is a graphical representation of the qRT-PCR analysis of mRNA of LDHA, LDHB, LDHC, and HK2 in WT and NAC1 KO B16-OVA cells. FIG. 8C is a graphical representation of the qRT-PCR analysis of mRNA of LDHA, LDHB, LDHC, and HK2 in WT and NAC1 KO A2058 cells. The results are presented relative to the level of GAPDH. FIG. 8D is a photographical representation of the Western blot analyses of expression of LDHA in whole-cell extracts from WT and NAC1 KO B16-OVA cells (left) and WT and NAC1 KO A2058 cells (right). β-actin was used as a loading control. One representative experiment out of three is shown. FIG. 8E and FIG. 8F are graphical representations of the correlations of NACC1 with LDHA gene expression (Z-score) as determined in a dataset including 470 melanoma tumors and 368 melanoma-metastatic tumors, respectively (‘R2: Tumor Skin Cutaneous Melanoma-TCGA-470-rsem-tcgars’). Pearson's correlation was calculated. FIG. 8G and FIG. 8H are graphical representations of the correlations of CD8⁺ T cell infiltration with NACC1 expression in melanoma (SKCM, n=470) and metastasis melanoma (SKCM-metastasis, n=368). FIG. 8I and FIG. 8J are graphical representations of the correlations of CD8⁺ T cell infiltration level with LDHA expression in melanoma (SKCM, n=470) and metastasis melanoma (SKCM-metastasis, n=368).

FIGS. 9A-9Z and 9AA-9ZZ demonstrate that effects of LDHA expression and lactic acid level on cytokine production, apoptosis, and exhaustion of CD8⁺ T cells. Mouse or human CD8⁺ T cells were incubated with CM from mouse WT or NAC1 KO B16-OVA cells, or human WT or NAC1 KO A2058 tumor cells for 24 hours. In parallel, some T cells were incubated with CM from NAC1 KO tumor cells overexpressing LDHA (NAC1 KO LDHA OE) or an empty control (NAC1 KO Mock), or CM supplemented with LA (2-5 mM for mouse T cells; 5-10 mM for human T cells). FIGS. 9A-9D are graphical representations of the production of cytokines TNF-α (FIG. 9A) or IFN-7 (FIG. 9B) and expression of PD-1 (FIG. 9C) and TIM-3 (FIG. 9D) of mouse CD8⁺ T cells with CM from mouse WT or NAC1 KO B16-OVA cells, or CM supplemented with LA by flow cytometry. FIGS. 9E-9H are representations of the flow cytometry analysis of the apoptosis of mouse CD8⁺ T cells with CM from mouse WT (FIG. 9E) or NAC1 KO B16-OVA cells (FIG. 9F), or CM supplemented with LA (FIG. 9G and FIG. 9H). FIGS. 9I-9L are graphical representations of the production of cytokines IFN-7 (FIG. 9I) or TNF-α (FIG. 9J) and expression of Granzyme B (FIG. 9K) and PD-1 (FIG. 9L) of mouse CD8⁺ T cells with CM from mouse WT or NAC1 KO B16-OVA cells, or CM from NAC1 KO tumor cells overexpressing LDHA or an empty control by flow cytometry. FIGS. 9M-9P are representations of the flow cytometry analysis of the apoptosis of mouse CD8⁺ T cells with CM from mouse WT (FIG. 9M) or NAC1 KO B16-OVA cells (FIG. 9N), or CM from NAC1 KO tumor cells overexpressing LDHA (FIG. 9O) or an empty control (FIG. 9P) by Annexin V staining. FIGS. 9Q-9X are representations of the analysis of the apoptosis of human CD8⁺ T cells with CM from human WT (FIG. 9Q and FIG. 9U) or NAC1 KO A2058 cells (FIG. 9R and FIG. 9V), or CM supplemented with 5 mM LA (FIG. 9S) or 10 mM LA (FIG. 9T), or CM from human NAC1 KO tumor cells overexpressing LDHA (FIG. 9W) or an empty control (FIG. 9X) by Annexin V staining. FIGS. 9Y-9FF are representations of the analysis of the production of IFN-γ of human CD8⁺ T cells with CM from human WT (FIG. 9Y and FIG. 9CC) or NAC1 KO A2058 cells (FIG. 9Z and FIG. 9DD), CM supplemented with 5 mM LA (FIG. 9AA) or 10 mM LA (FIG. 9BB), or CM from human NAC1 KO tumor cells overexpressing LDHA (FIG. 9EE) or an empty control (FIG. 9FF). FIGS. 9GG-9NN are representations of the analysis of the production of IL-2 of human CD8⁺ T cells with CM from human WT (FIG. 9GG and FIG. 9KK) or NAC1 KO A2058 cells (FIG. 9HH and FIG. 9LL), CM supplemented with 5 mM LA (FIG. 9II) or 10 mM LA (FIG. 9JJ), or CM from human NAC1 KO tumor cells overexpressing LDHA (FIG. 9MM) or an empty control (FIG. 9NN). FIGS. 9OO-9VV are representations of the analysis of the expression of PD-1 of human CD8⁺ T cells with CM from human WT (FIG. 9OO and FIG. 9SS) or NAC1 KO A2058 cells (FIG. 9PP and FIG. 9TT), CM supplemented with 5 mM LA (FIG. 9QQ) or 10 mM LA (FIG. 9RR), or CM from human NAC1 KO tumor cells overexpressing LDHA (FIG. 9UU) or an empty control (FIG. 9VV). FIG. 9WW is a graphical representation of the ECAR of mouse tumor cells measured by the Seahorse Metabolic Analyzer. FIG. 9XX is a graphical representation of basal glycolysis rates (left panel) and maximum glycolysis rate (right panel) of mouse tumor cells. One-way ANOVA with multiple comparisons correction. ***p<0.001. FIG. 9YY is a graphical representation of ECAR of human tumor cells measured by the Seahorse Metabolic Analyzer. Data are represented as mean±SEM; n=6 per condition from two independent experiments. FIG. 9ZZ is a graphical representation of basal glycolysis rates (left panel) and maximum glycolysis rate (right panel) of human tumor cells.

FIGS. 10A-10M demonstrate NAC1 deficiency increases CTL infiltration in tumors following an ACT in immune-competent mice. Mouse WT B16-OVA or NAC1 KO B16-OVA tumor cells (1×106) were injected s.c. in the flank of B6. Thy1.1⁺ mice (n=5), followed by treatment with or without the ACT of OT-I CD8⁺ T cells. FIG. 10A is a graphical representation of the progression of tumor size from mouse WT B16-OVA or NAC1 KO B16-OVA tumor cells, followed by treatment with or without the ACT of OT-I CD8⁺ T cells. FIG. 10B is a set of immunofluorescence DAPI, CD8, Thy1.2, and a merged staining of OT-I CD8⁺ T cell infiltration (Thy1.2⁺CD8⁺) in tumors. FIG. 10C is a set of photographic images of the indicated tumors as harvested on day 23. FIG. 10D is a flow cytometric analysis of tumor-infiltrating CD4⁺ T cells and CD8⁺ T cells from WT B16-OVA or B16-OVA NAC1 KO mice, followed by treatment with or without the ACT of OT-I CD8⁺ T cells. FIGS. 10E and 10F are graphical representations the percentage of tumor-infiltrating CD4⁺ T cells and CD8⁺ T cells from WT B16-OVA or B16-OVA NAC1 KO mice, followed by treatment with or without the ACT of OT-I CD8⁺ T cells. FIGS. 10G and 10H are histograms showing expression of PD-1⁺ (FIG. 10G) and quantitation of PD-1⁺ cells of total infiltrated Thy1.2⁺ CD8⁺ cells (FIG. 10H). FIGS. 10I and 10J are histograms showing production of IL-2 (FIG. 10I) and quantitation of MFI of IL-2 of total infiltrated CD8⁺ cells (FIG. 10J). FIGS. 10K and 10L are histograms showing production of Granzyme B (FIG. 10L) and quantitation of MFI of Granzyme B of total infiltrated CD8⁺ cells (right panel). FIG. 10M is a schematic representation of experimental paradigm. B6 Thy 1.1 mice were injected with mouse or human tumor cells on Day 1 followed by antigen specific CD8⁺ T cell transfer on day 7. The tumor size was monitored until day 23 post injection of the melanoma cells. All the results shown are representative of three identical experiments.

FIGS. 11A-11O demonstrate NAC1 deficiency increases tumor CTL infiltration following an ACT in immune-deficient mice. Human WT A2058 or NAC1 KO A2058 tumor cells (1×106) were s.c. injected into the flank of NSG mice, followed by treatment with or without the ACT of Tyrosinase-specific CD8⁺ T cells. FIG. 11A is a graphical representation of the progression of tumor size. FIG. 11B is a graphical representation of the tumor weight as evaluated by scales and represented graphically. Values are mean±SEM of three independent experiments (n=5). FIG. 11C is a set of photographs of the indicated tumors as harvested and photographed at day 25. FIG. 11D is a set of photographs of the CD8⁺ T cell infiltration (CD8⁺; green) in tumors (tyrosinase; red) as determined by immunofluorescence staining. Images are representative images taken from the tumors of three different mice. FIGS. 11E-11H are flow cytometric analysis of infiltrating CD8⁺ T cells in WT (FIGS. 11E and 11F) and NAC1 KO mice (FIGS. 11G and 11H) with or without the ACT of Tyrosinase-specific CD8⁺ T cells, respectively. FIG. 11I is a graphical representation of the tumor infiltrating CD8⁺ T cells in WT and NAC1 KO mice with or without the ACT of Tyrosinase-specific CD8⁺ T cells, respectively. Values are the mean±SEM of 3 independent experiments (n=5). FIG. 11J is a graphical representation of the flow cytometric analysis of infiltrating TIM-3⁺ CD8⁺ T cells. FIG. 11K is a graphical representation of the flow cytometric analysis of infiltrating PD-1⁺CD8⁺ T cells. Values are the mean±SEM of 3 independent experiments (n=5). FIG. 11L is a graphical representation of the flow cytometric analysis of IFN-7 production of infiltrating CD8⁺ T cells. FIG. 11M is a graphical representation of the flow cytometric analysis of the IL-2 production of infiltrating CD8⁺ T cells. FIG. 11N is a graphical representation of the flow cytometric analysis of Granzyme B production of infiltrating CD8⁺ T cells. Values are the mean±SEM of four independent experiments (n=4). FIG. 11O is a schematic representation of the mechanism.

FIGS. 12A-12B demonstrate that NAC1^{-/-} Tregs show enhanced suppressive function. WT or NAC1^{-/-} Tregs (1×105) were injected s.c. in the flank region of the recipient mice with 1×106 B16 tumor cells on various days. FIG. 12A is a graphical representation of the tumor growth following injection of WT or NAC1^{-/-} Tregs. Data shown are the mean±S.E.M. of tumor sizes of a representative of three identical experiments (N=6). ***, P<0.0001, simple linear regression. FIG. 12B is a graphical representation of the survival curves following injection of WT or NAC1^{-/-} Tregs.

FIGS. 13A-13D illustrate the defects in glycolysis and oxidative phosphorylation rate of NAC1^{-/-} CD8⁺ T cells. Cell metabolism was tested using the Agilent Seahorse Assay on day 3 after activation. (A, C) The cells were activated with coated anti-CD3 and soluble anti-CD28 Abs. Then, 2×10⁵ cells were plated in each well of seahorse microplate for glycolytic rate testing. The drugs were injected into each well at the time indicated in the figures. FIG. 13A is a graphical representation of the Extra Cellular Acidification Rate (ECAR), which was measured as a proxy for glycolytic rate. FIG. 13C is a graphical representation of the ECAR level after ROT/AA injection as compared between WT and NAC1^{-/-} groups. Sample numbers (n=4), the p-value is 0.0076. The activated 2×105 T cells were seeded in a microplate before mitochondrial stress testing. The different drugs were added to the well according to the indicated time points as shown in FIG. 13B. FIG. 13B is a graphical representation of the Oxygen Consumption Rate (OCR), which was tested as a proxy for oxidative phosphorylation. The OCR level was then compared after FCCP injection between the two groups. FIG. 13D is a graphical representation of the OCR level after FCCP injection as compared between WT and NAC1^{-/-} groups.

FIGS. 14A-141 provide the comparison of development of viral Ag-specific CD8⁺ T cells. The VACV-specific CD8⁺ T cell frequency was monitored in WT or NAC1^{-/-} mice for 35 days after VACV challenge. Mice were challenged with VACV at 2×10⁶ PFU/mouse. The spleen and LNs (superficial cervical, axillary, brachial, and inguinal nodes) were dissected, smashed, and stained with CD8 Ab and B8R tetramer. On day 7, day 14, day 21, and day 35, Ag-specific cell frequencies were analyzed by flow cytometry. FIGS. 14A-14H are flow cytometry analyses of CD8 Ab and B8R tetramer in WT (FIGS. 14A-14D) or NAC1^{-/-} mice (FIGS. 14E-14H) on day 7, day 14, day 21, and day 35 after VACV challenge. VACV-uninfected mice were used as a control. The spleen and lymph nodes were also dissected, smashed, and stained with CD8 Ab and B8R tetramer. FIG. 14I is a flow cytometry analysis of CD8 Ab and B8R tetramer in VACV-uninfected mice.

FIGS. 15A-15B demonstrate the sustained VACV-specific CD8⁺ T cell survival in NAC1^{-/-} mice. Mice were challenged with VACV at 2×10⁶ PFU/mouse. The spleen and lymph nodes (superficial cervical, axillary, brachial, and inguinal nodes) were dissected and smashed. The total live cell number for each mouse was calculated with trypan blue staining using a Bio-Rad cell counter. T cells were stained with CD8 Ab and B8R tetramer and analyzed by flow cytometry. Total Ag-specific CD8⁺ T cells were calculated for each mouse (n=5). FIG. 15A is a graphical representation of the VACV-specific CD8⁺ T cell number during the 35 days post-infection. The p-values are 0.00038 (day 7), 8.2×10⁻⁵ (day 14), and 0.0066 (day 21). The Ag-specific CD8⁺ T cell frequency was monitored for 35 days. FIG. 15B is a graphical representation of the VACV-specific CD8⁺ T cell frequency during the 35 days post-infection. P-values are 0.011 (day 7), and 0.016 (day 21).

FIGS. 16A-16C provide analyses of CD8⁺ T cell memory formation. T cell memory population was investigated in WT or NAC1^{-/-} mice after VACV challenge. The mice were sacrificed on day 35. The spleen and LNs were dissected, smashed, and stained before flow cytometry. FIG. 16A is a flow cytometry analysis of memory CD8⁺ T cells (CD8⁺ CD44⁺) in WT (top) or NAC1^{-/-} mice (bottom) after VACV challenge. FIG. 16B is a flow cytometry analysis of VACV-specific memory CD8⁺ T cells (CD8⁺ CD44⁺ B8R⁺), gating on memory CD8⁺ T cells, in WT (top) or NAC1^{-/-} mice (bottom) after VACV challenge. FIG. 16C is a flow cytometry analysis of tissue-resident memory (CD44^{hi}CD69^{hi}CD197^{low}), central memory (CD44^{hi}CD69^{low}CD197^{hi}) and effector memory (CD44^{hi}CD69^{low}CD197^{low}) T cells, gating on VACV-specific memory CD8⁺ T cells, in WT (top) or NAC1^{-/-} mice (bottom) after VACV challenge.

FIGS. 17A-17C illustrate the enhanced CD8⁺ T cell memory formation in NAC1^{-/-} mice. Quantification for T cell memory population after 35 days. The mice were sacrificed on day 35. The spleen and lymph nodes were dissected and smashed. The total live cell number for each mouse was calculated with trypan blue staining through a Bio-Rad cell counter. T cell surface markers were stained before flow cytometry. FIG. 17A is a graphical representation of the VACV-specific memory CD8⁺ T cell (CD8⁺ CD44⁺ B8R⁺) number for each mouse, as calculated and compared between WT and NAC1^{-/-} groups. FIG. 17B is a graphical representation of the VACV-specific memory CD8⁺ T cell (CD8⁺ CD44⁺ B8R⁺) frequency for each mouse, as calculated and compared between WT and NAC1^{-/-} groups. The p-value is 0.040. FIG. 17C is a graphical representation of the tissue-resident memory (CD44^{hi}CD69^{hi}CD197^{low}) T cell frequency when analyzed between WT and NAC1^{-/-} groups.

FIGS. 18A-18C illustrate the regulation of IRF4 in CD8⁺ T cells by NAC1. CD8⁺ T cells were isolated from WT or NAC1^{-/-} mice and analyzed for expression of IRF4. FIG. 18A is a photographic representation of the Western blot analysis of IRF4 expression. For the day 0 sample, T cells were not activated. For the day 3 sample, T cells were activated and cultured for 3 days. FIG. 18B is a photographic representation of the Q-PCR analysis of mRNA expression. CD8⁺ T cells had been cultured for 3 days, and then RNA was extracted, and qPCR was performed (ns, p>0.05). FIG. 18C is a representation of the CHIP-seq analysis. WT CD8⁺ T cells were isolated from mice and CHIP was performed with anti-NAC1 and anti-IgG Abs (Input control). The sequenced data was visualized by IGV.

### DETAILED DESCRIPTION

The present disclosure describes various embodiments related to compositions and methods for management or treatment of autoimmune disorders.

Embodiments include methods for enhancing or inducing an anti-tumor response in a subject by administering to the subject a therapeutically effective amount of an inhibitor of expression or activity of NAC1. The anti-tumor response can be an increase in CD8⁺ T cell-mediated anti-tumor immunity or a persistent anti-tumor T cell memory. In certain embodiments, the subject has been administered an adoptive cell transfer therapy, such as a chimeric antigen receptor T-cell therapy or a tumor-infiltrating lymphocyte therapy. In certain embodiments, the subject has a solid tumor, such as a melanoma. In certain embodiments, the subject has a solid tumor, such as a carcinoma or a sarcoma. Effective amounts of the inhibitor of NAC1 improve the tumor microenvironment through suppression of tumor cell metabolism and increases CD8⁺ T cell-mediated anti-tumor immunity. Methods also include administering to the patient an effective amount of NAC1-targeted siRNA nanoliposomes or CRISPR/Cas9 for enhancing or inducing an anti-tumor immune response including persistent anti-tumor T cell memory in the patient. The NAC1-targeted siRNA nanoliposomes or CRISPR/Cas9 compositions improve the tumor microenvironment through suppression of tumor cell metabolism and increase CD8⁺ T cell-mediated anti-tumor immunity. These chemical and biological agents that target NAC1 are also provided as adjuvants to T-cell-based immunotherapy.

Embodiments include methods of treating an autoimmune disorder by administering a therapeutically effective amount of an inhibitor of NAC1. In certain embodiments, the autoimmune disorder is autoimmune arthritis. In certain embodiments, the autoimmune disorder is autoimmune colitis.

Embodiments include methods of enhancing effectiveness of a vaccine in a subject by administering to the subject a therapeutically effective amount of an inhibitor of NAC1. The inhibitor of NAC1 can be administered before, after or concurrent with the vaccine. The vaccine can be a COVID-19 vaccine, an influenza vaccine, a human papillomavirus vaccine, a hepatitis A or B vaccine, or a tumor vaccine.

Embodiments of inhibitors of expression of NAC1 include compositions containing a NAC1-targeted siRNA as an inhibitor of NAC1. The NAC1-targeted siRNA can have high silencing activity of NAC1. For example, the siRNA sequence can be of:

SEQ ID NO. 1:
5′-UGAUGUACACGUUGGUGCCUGUCACCA-3′
or
SEQ ID NO. 2:
5′-UGUAGCAGAAGCUGAGGAUCUGCUG-3′.

The NAC1-targeted siRNA can be administered as a nanoliposome. The inhibitor of NAC1 can be a CRISPR/Cas-based genome editing composition comprising one or more vectors encoding: (a) one or more guide RNAs (gRNAs) that are complementary to one or more target sequences in a NAC1 gene and (b) a nucleic acid sequence encoding a Clustered Regularly Interspaced Short Palindromic Repeat (CRISPR)-associated endonuclease, whereby the one or more gRNAs, hybridize to the NAC1 gene and the CRISPR-associated endonuclease cleaves the NAC1 gene. The NAC1 sequence can be deleted in the patient relative to a patient to whom the CRISPR/Cas-based genome editing composition is not administered. The NAC1 expression or activity can be reduced in the patient relative to a patient to whom the CRISPR/Cas-based genome editing composition is not administered. The inhibitor of NAC1 can be an isolated antibody or its binding fragment thereof that binds to NAC1. The inhibitor of NAC1 corresponds to Formula I.

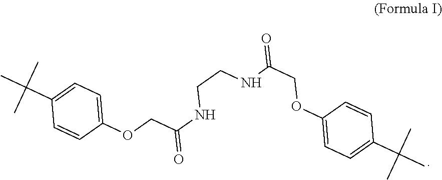 [SMILES: CC(C)(C)c1ccc(OCC(=O)NCCNC(=O)COc2ccc(C(C)(C)C)cc2)cc1]

In the following description, numerous details are set forth in order to provide a thorough understanding of the various embodiments. Before the present methods and compositions are described, it is to be understood that these embodiments are not limited to particular methods or compositions described, as such may, of course, vary. It is also to be understood that the terminology used herein is for the purpose of describing particular embodiments only, and is not intended to be limiting, as the scope of the present embodiments will be limited only by the appended claims. The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

A “patient” or a “subject” refers to an animal, such as a mammal, including a primate (such as a human, a non-human primate, e.g., a monkey) and a non-primate (such as a mouse). In some aspects, the patient or the subject is a human. In some aspects, the patient is a pediatric patient, such as a neonate, an infant, or a child. In other aspects, the patient is an adult patient.

A “therapeutically effective amount” is an amount sufficient to effect desired clinical results (i.e., achieve therapeutic efficacy). A therapeutically effective dose can be administered in one or more administrations. “Administering” refers to the physical introduction of a therapeutic agent to a patient in need thereof. Exemplary routes of administration for agents to inhibit NAC1 include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent may be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Therapeutic agents can be constituted in a composition, e.g., a pharmaceutical composition containing a chemical compound and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible.

As used herein, the terms “treating”, “treatment” and the like, shall include the management and care of a subject or patient for the purpose of combating a disease, condition, or disorder and includes the administration of a composition to prevent the onset of the symptoms or complications, alleviate the symptoms or complications, reduce at least one associated sign, symptom, or condition, or eliminate the disease, condition, or disorder. Treatment also refers to a prophylactic treatment, such as prevention of a disease (e.g., autoimmune disorders) or prevention of at least one sign, symptom, or condition associated with the disease (e.g., autoimmune disorders), such as increasing the effectiveness or efficacy of a vaccine. Treatment can also mean prolonging survival as compared to expected survival in the absence of treatment.

Nucleus accumbens-associated protein-1 (NAC1), encoded by NACC1 gene was identified as a vital modulator of immune suppression. NAC1 is a nuclear factor that belongs to the BTB (Broad-Complex, Tramtrack and Bric a brac)/POZ (POX virus and Zinc finger) gene family. Using NAC1′ mice, NAC1 was identified as key in triggering autoimmunity and Treg instability. NAC1 contributes to break of immune tolerance through its negative control of Treg development and function associated with deacetylation and destabilization of FoxP3 protein.

Provided herein are methods of treating an autoimmune disorder by administering a therapeutically effective amount of an inhibitor of NAC1. The autoimmune disorder can be an autoimmune arthritis. The autoimmune disorder can be an autoimmune colitis, such as ulcerative colitis. Embodiments of an inhibitor of NAC1 include both chemical and biological agents that inhibit the function of NAC1. Certain embodiments include an inhibitor of NAC1 corresponding to Formula I (referred to as NIC3):

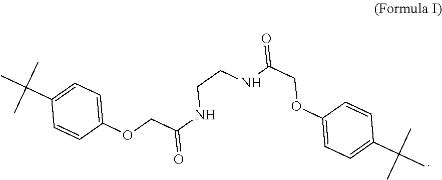 [SMILES: CC(C)(C)c1ccc(OCC(=O)NCCNC(=O)COc2ccc(C(C)(C)C)cc2)cc1]

Methods of treating an autoimmune disorder also include administering to the patient an effective amount of NAC1-targeted siRNA nanoliposomes or CRISP/Cas9 constructs.

Provided herein are methods of modulating an immune response in a patient by administering to the patient a therapeutically effective amount of an inhibitor of NAC1. In certain embodiments, administration of the NAC1 inhibitor modulates the immune response mediated by regulatory T cells. In certain embodiments, administration of the NAC1 inhibitor decreases the level of an immune response in a patient. Embodiments include both chemical and biological agents that inhibit the function of NAC1. Certain embodiments include a composition containing NIC3. Certain embodiments include a composition containing an isolated antibody or its binding fragment thereof that binds to NAC1. In some embodiments, expression of one or more allele(s) of the NAC1 gene is reduced in the cancer cell. In some embodiments, NAC1 activity is reduced in the cancer cell. In some embodiments, NAC1 expression or activity is not completely eliminated in the cancer cell. In some embodiments, NAC1 expression or activity is completely eliminated in the cancer cell.

FoxP3⁺ regulatory T cells (Tregs) are a distinct subset of CD4⁺ T cells integral to the maintenance of the balance of the immune system, and their dysregulation is a trigger of autoimmunity. NAC1 is a negative regulator of FoxP3 in Tregs and a critical determinant of immune tolerance. Phenotypically, NAC1^{-/-} mice show substantial tolerance to the induction of autoimmunity, as evidenced by the significantly decreased occurrences of autoimmune arthritis and colitis. Analysis of T cells from the wild-type (WT) or NAC1 knockout (^{-/-}) mice found that NAC1 is crucially involved in the early stage of T cell development. NAC1 positively affects CD8⁺ T cell differentiation, but negatively regulates Treg development. Compared with WT animals, NAC1^{-/-} mice displayed defects in CD8⁺ T cell development but generated a larger amount of CD4⁺ regulatory Tregs that exhibit a higher metabolic profile and immune suppressive activity, increased acetylation, and expression of FoxP3, and slower turnover of this transcriptional factor. Furthermore, treatment of Tregs with the pro-inflammatory cytokines IL-10 or TNF-α induced a robust upregulation of NAC1 but an evident downregulation of FoxP3 as well as the acetylated FoxP3, demonstrating that the reduction of FoxP3 by the NAC1-mediated deacetylation and destabilization of this lineage-specific transcriptional factor contributes considerably to break of immune tolerance. The pro-inflammatory cytokines-stimulated upregulation of NAC1 acts as a trigger of the immune response through destabilization of Tregs and suppression of tolerance induction. Therapeutic targeting of NAC1 by chemical or biological agents is a tolerogenic strategy for treatment of autoimmune disorders.

### Overall T Cell Population is Divergent in the WT and NAC1^{-/-} Mice

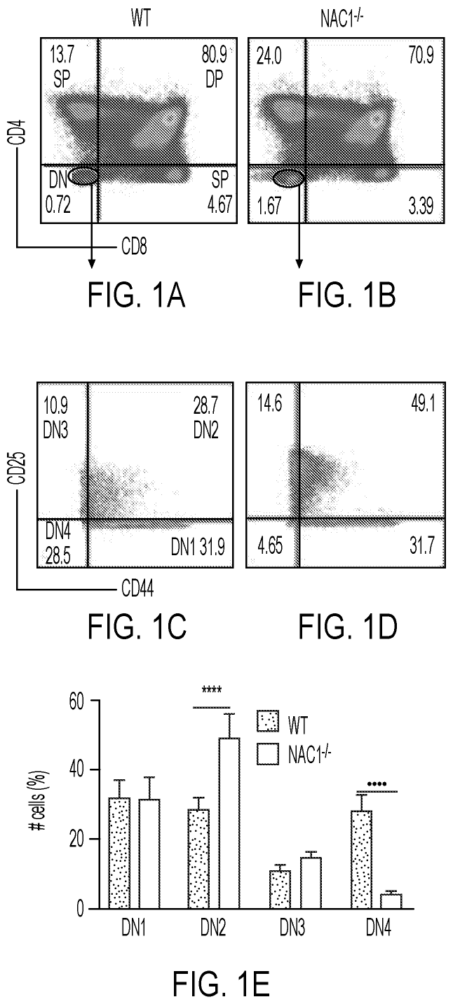

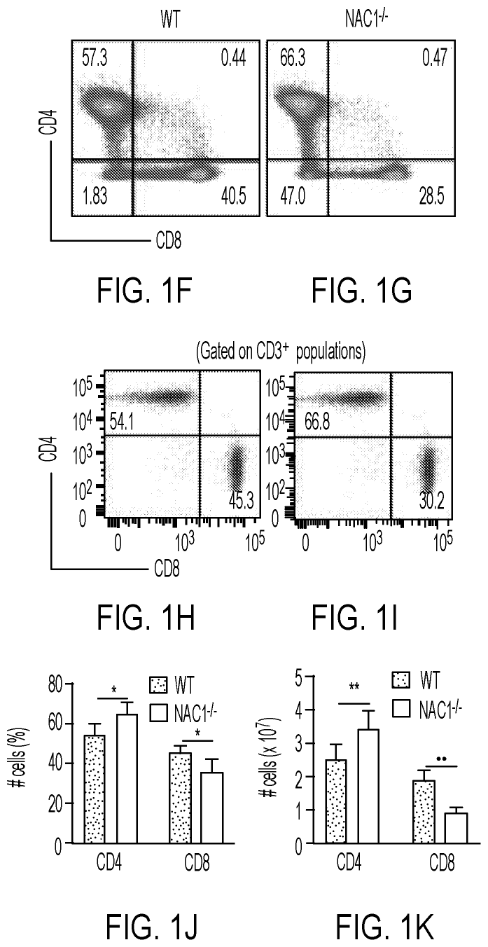

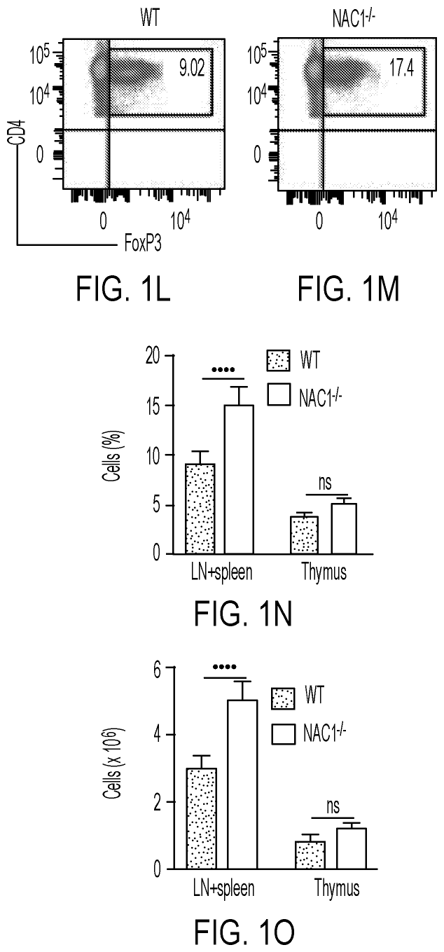

NAC1 participates in regulation of the self-renewal and pluripotency of embryonic stem cells and somatic cell reprogramming. NAC1 has a critical role in cellular metabolism. As metabolic reprogramming can significantly influence T cell activation, expansion, and effector function, NAC1's effects on T cell development and function were examined. T cell profiling in WT and NAC1^{-/-} mice was first performed. Compared with WT mice, development of T cells in the thymus of NAC1^{-/-} mice was curbed, as evidenced by increased numbers of thymocytes in the dominant-negative (DN) stage (1.67% vs. 0.72%) and decreased numbers of cells in the DN4 stage (4.65% vs. 28.5%; p<0.0001) (FIGS. 1A-1D). Although NAC1^{-/-} mice showed a reduction of total thymocytes and decreased cell amount in the DN4 stage, an accumulation of T cells in the DN2 stage was observed in those animals (49.1.5% vs. 28.7%; p<0.0001) (FIGS. 1A-1E). These alterations were correlated with a decreased percentage of TCRβ⁺ cells in DN4 cells found in NAC1^{-/-} mice. Despite a higher % of DN cells in NAC1^{-/-} mice, which show higher numbers of CD117 cells, WT animals had a greater number of TCRβ⁺ cells than NAC1^{-/-} mice. Like the gating on the DN4 population, there was a decrease in TCRβ⁺ cells in NAC1^{-/-} mice as compared with WT animals, which could be due to a higher % of DN4 cells in WT mice. Conversely, in the lymph nodes (LNs) and spleen of NAC1^{-/-} mice, there was a reduced percentage of CD8⁺ T cells (28.5% vs. 40.5%) but an increased percentage of CD4⁺ T cells (66.3% vs. 57.3%) as compared to the controls (FIGS. 1F-1I). Despite the similar numbers of the total CD4⁺ or CD8⁺ single positive (SP) T cells in the LNs and spleen, there was a significant increase of % CD4⁺ SP T cells (p<0.05) but a decrease of CD8⁺ SP T cells (p<0.01) in NAC1^{-/-} mice, as compared with WT controls (FIGS. 1J-1K). These results indicate the important role for NAC1 in the early stage of T cell development and in the differentiation of CD4⁺ and CD8⁺ SP cells.

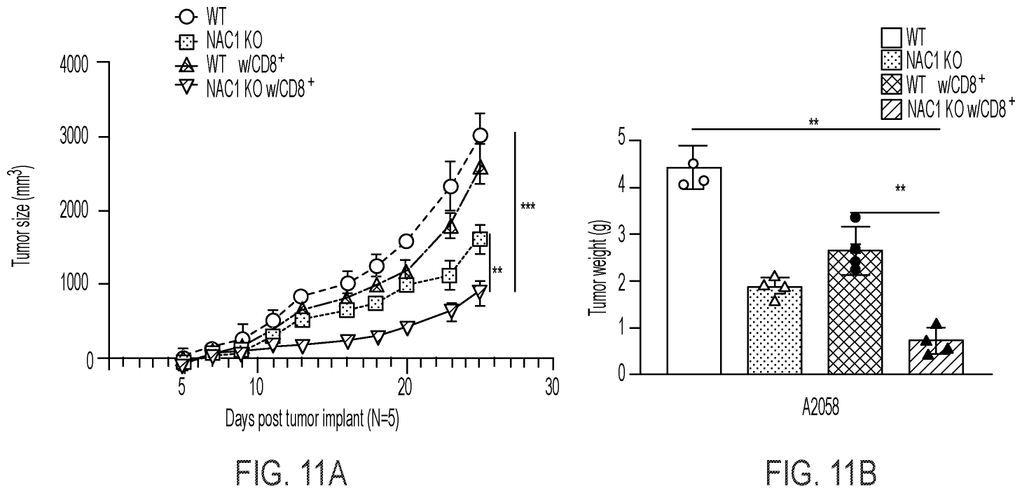

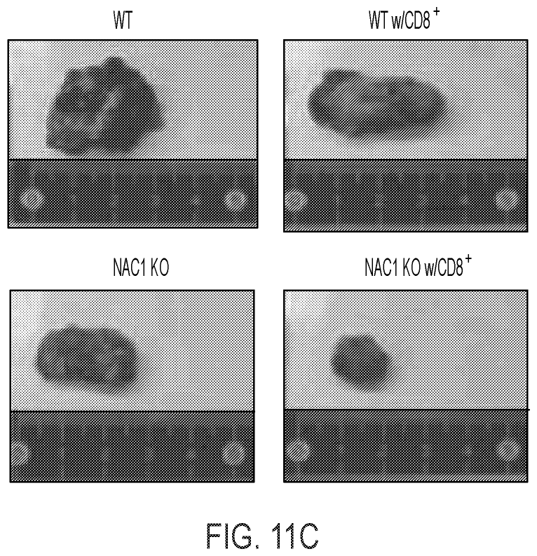

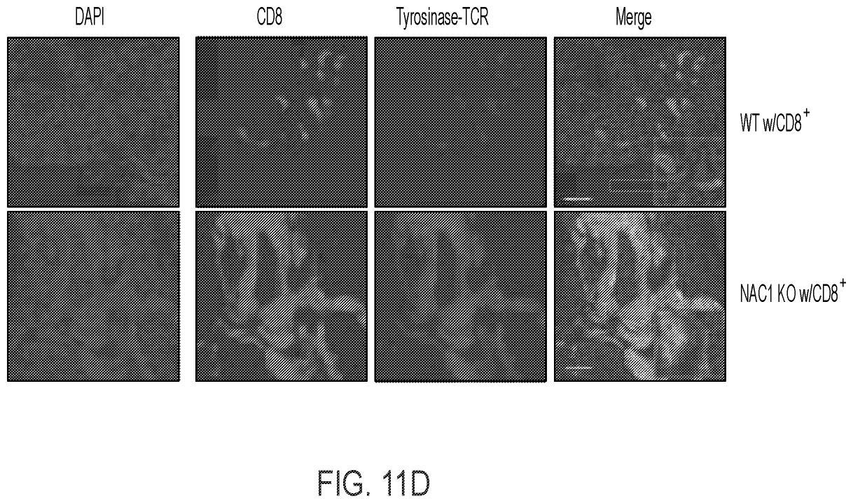

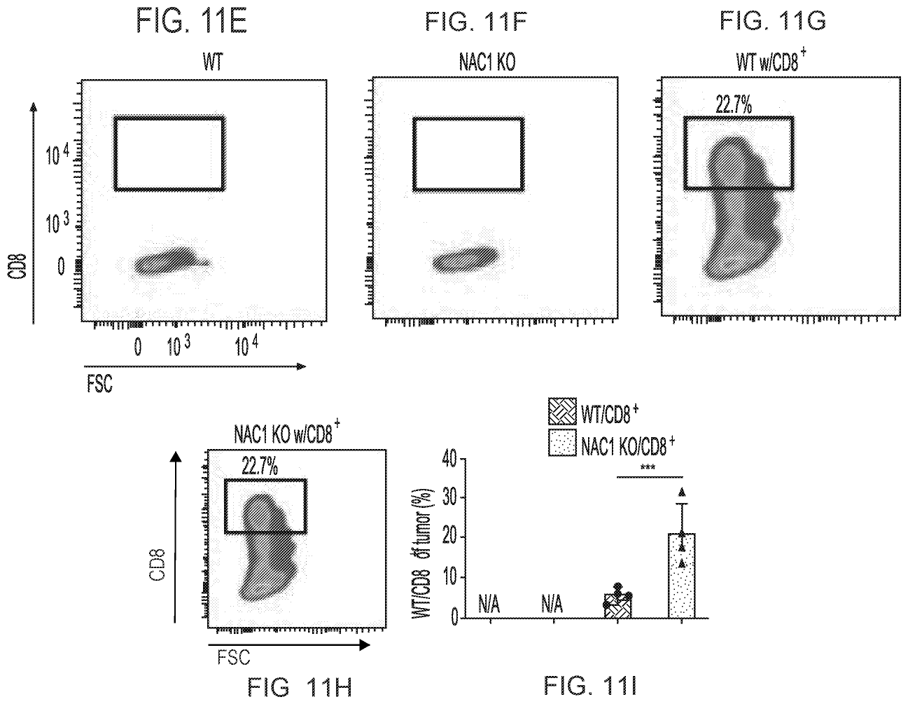

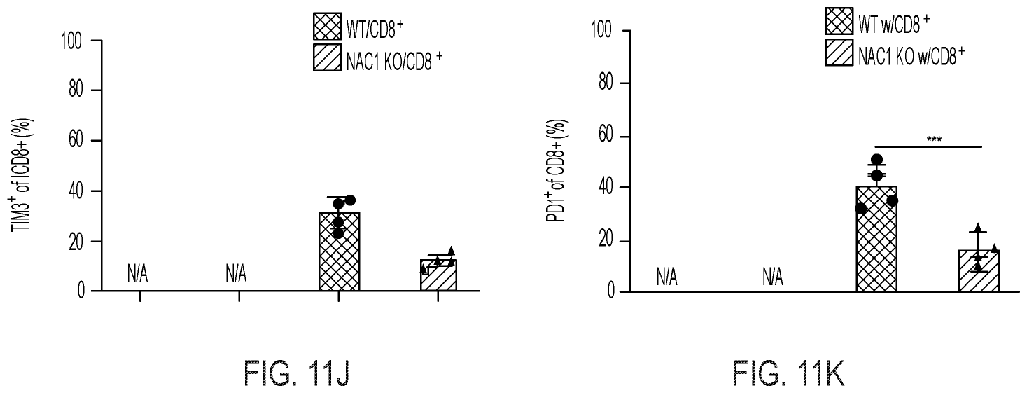

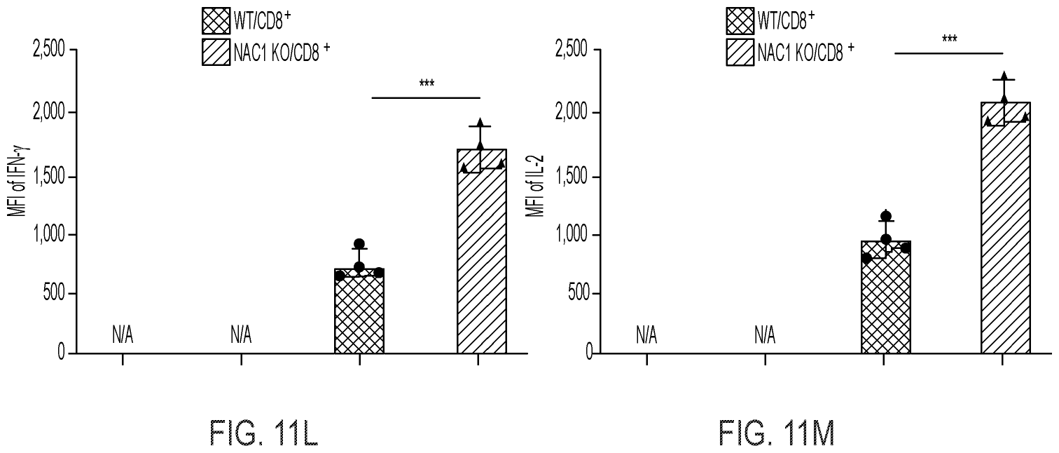

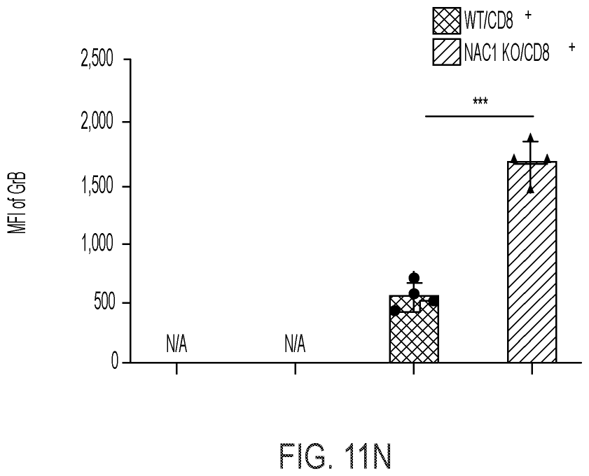

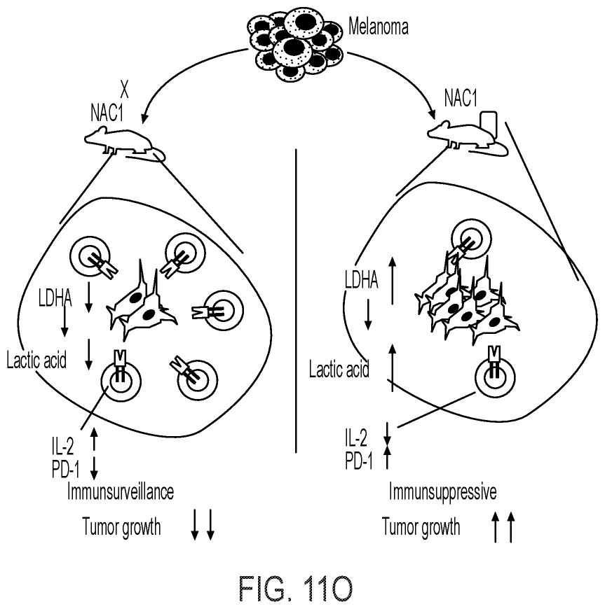

FIGS. 1A-1O demonstrates the impact of the loss of NAC1 on overall T cell populations. T cells from the thymus, peripheral lymph nodes (LNs) and spleen of WT or NAC1 mice were analyzed by flow cytometry and calculated for numbers or percentages. FIGS. 1A and 1B are flow cytometry analyses of the CD4 and CD8 in the thymus of WT and NAC1^{-/-} mice. The double negative (DN) populations (shown as circles with arrows pointing outward) were analyzed for DN1 to DN4 stages based on CD44 and CD25. FIGS. 1C and 1D are flow cytometry analyses of for DN1 to DN4 stages based on CD44 and CD25. Data shown are the representative of five mice per group of three independent experiments. FIG. 1E is a graphical representation of the percentages of total thymocytes, CD4 or CD8 single positive (SP) and the percentages of DN2 or DN4 cells from WT and NAC1^{-/-} mice. Data shown are the representative of three identical experiments. The values represent mean±S.D. (N=4 or 5). ****, p<0.0001, unpaired t-test. FIGS. 1F and 1G are flow cytometry analyses of the CD4 and CD8 in the LNs and spleen of WT and NAC1^{-/-} mice. FIGS. 1H and 11 are flow cytometry analyses of the CD4 and CD8 cells gated on CD3⁺ populations in the LNs and spleen of WT and NAC1^{-/-} mice. Data shown are the representative of five mice per group of three independent experiments. ***, p<0.001, unpaired t-test. FIGS. 1J and 1K are graphical representations of percentages of CD4 and CD8 T cells the LNs and spleen, respectively, of WT and NAC1^{-/-} mice.

### Deficiency of NAC1 Promotes Treg Development and Stability

The significant increase of total peripheral CD4⁺ T cell population observed in NAC1^{-/-} mice (FIGS. 1L and 1M) led to the investigation of the regulatory role of NAC1 in the development of Tregs, a unique sub-type of CD4⁺ T cells able to suppress excessive immune reaction. Indeed, compared with WT animals, NAC1^{-/-} mice showed a significant increase in % of Tregs in the LNs and spleen (p<0.0001; (FIGS. 1L and 1M). Moreover, NAC1^{-/-} animals had significantly higher % and numbers of Tregs in the LNs and spleen but not in the thymus ((FIGS. 1N and 1O). FIGS. 1L and 1M are flow cytometry analyses of the CD4 and FoxP3 cells in WT and NAC1^{-/-} mice, respectively. Data shown are the representative of five mice per group of three independent experiments. ***, p<0.001, unpaired t-test. FIGS. 1N and 1O are graphical representations of percentages of Tregs cells in the LNs+spleen and thymus of WT and NAC1^{-/-} mice, respectively. Data shown are representative of three identical experiments. The values represent mean±S.D. (N=4 or 5). ****, p<0.0001, unpaired t-test.

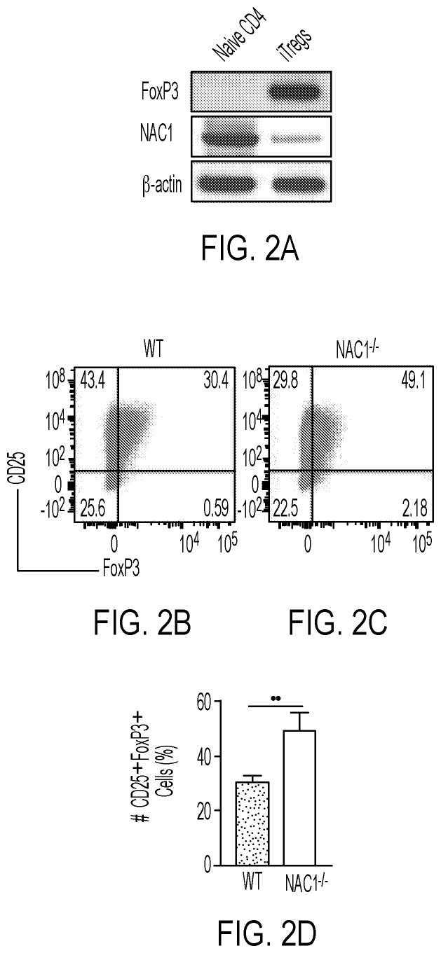

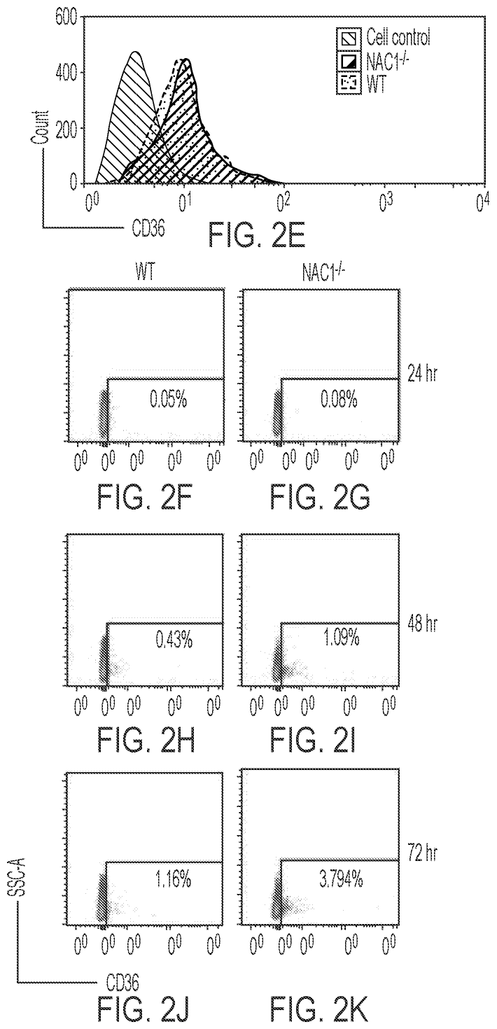

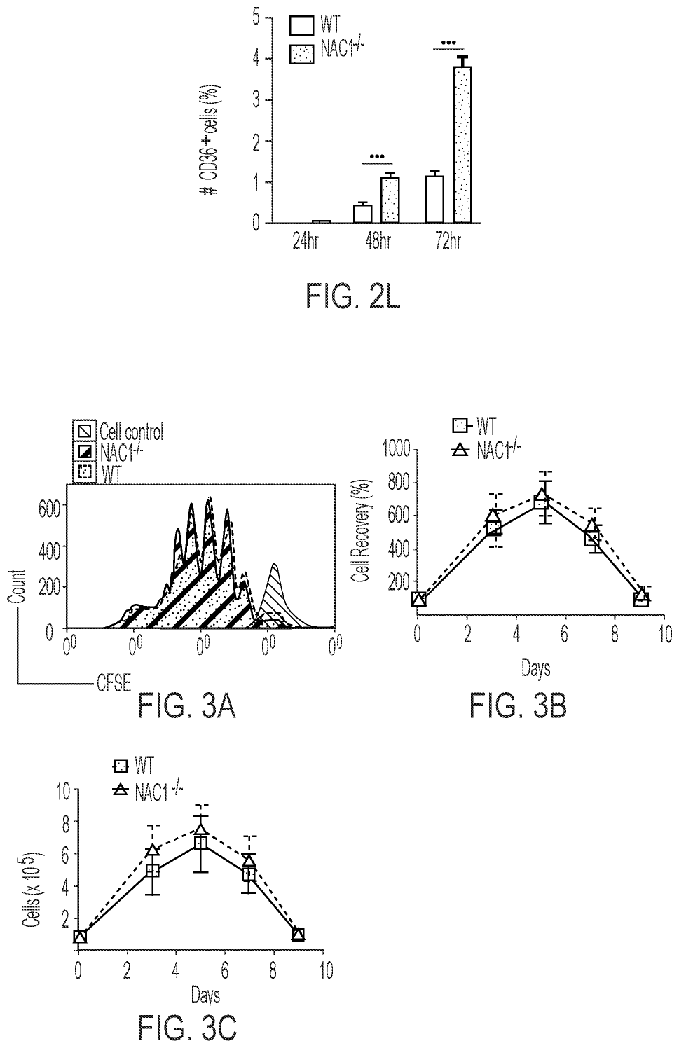

To prove the role of NAC1 in the development of Tregs, an in vitro system was used in which induced Tregs (iT_{regs}) are generated from naive CD4⁺CD25⁻ T cells. The naive CD4⁺CD25⁻ T cells from the LNs and spleen of WT or NAC1^{-/-} mice were treated with TGF-β to produce iT_{regs}. The naive CD4⁺CD25⁻ T cells from WT mice expressed abundant NAC1 but no detectable FoxP3; notably, the iT_{regs} from those T cells showed a robust expression of FoxP3 but a substantial reduction of NAC1 expression (FIG. 2A). Remarkably, a significantly greater amount of iT_{regs} were generated from NAC1^{-/-} than from WT CD4⁺CD25⁻ T cells (FIGS. 2B-2D).

FIGS. 2A-2L depict the loss of NAC1 enhancing the induction of iT_{regs} and expression of CD36. The naive CD4⁺CD25⁻ T cells from the pooled spleen and LNs of WT or NAC1^{-/-} mice were induced to iT_{regs} in the presence of TGF-β. FIG. 2A is a photograph of an immunoblot showing expression of NAC1 in naive CD4 and iT_{regs} of WT T cells. FIG. 2B and FIG. 2C are flow cytometry analyses of expression of CD25 and FoxP3. FIG. 2D is a graphical representation of the percentages of CD25⁺FoxP3⁺ populations in WT and NAC1^{-/-} mice. **, p<0.005, Student's unpaired t-test.

Furthermore, in response to stress the expression of CD36, an immuno-metabolic receptor that mediates metabolic adaptation and supports Treg survival and function, was significantly elevated in NAC1^{-/-} Tregs as compared with WT Tregs. FIG. 2E is a graphical representation of CD36 expression of untreated WT or NAC1^{-/-} Tregs when analyzed using flow cytometry. Tregs generated in vitro were treated with 10 mM lactic acid for various times and analyzed by flow cytometry for CD36 expression. FIG. 2F and FIG. 2G are flow cytometry analyses of expression of SSCA-A and CD36 in WT and NAC1^{-/-} mice, respectively, following 24 hours of lactic acid treatment. FIG. 2H and FIG. 2I are flow cytometry analyses of expression of SSCA-A and CD36 in WT and NAC1^{-/-} mice, respectively, following 48 hours of lactic acid treatment. FIG. 2J and FIG. 2K are flow cytometry analyses of expression of SSCA-A and CD36 in WT and NAC1^{-/-} mice, respectively, following 72 hours of lactic acid treatment. FIG. 2L is a graphical representation of the percentages of CD36⁺ Tregs following lactic acid treatment in WT and NAC1^{-/-} mice. The results shown are the mean S.E.M. of three identical experiments. *** P<0.001, Student's unpaired t-test. These results indicate that NAC1 exerts a negative control of Treg development at early stages. In addition to affecting CD4⁺ populations, NAC1 also affects CD8⁺ T cells. NAC1 deficiency led to a significant decrease of CD8⁺ SP T cell generation in the LNs and spleen, reduced production of cytokine, and shortened cellular survival. Viral Ag-stimulated development of memory CD8⁺ T cells was also suppressed in the absence of NAC1.

### NAC1^{-/-} Tregs Display Enhanced Functional Activities

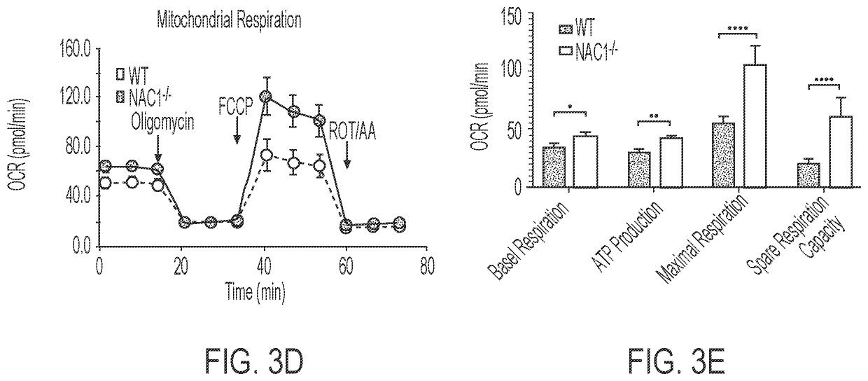

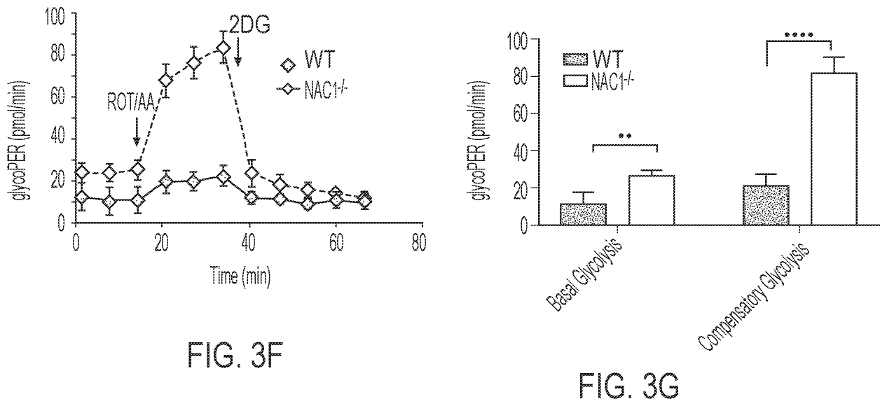

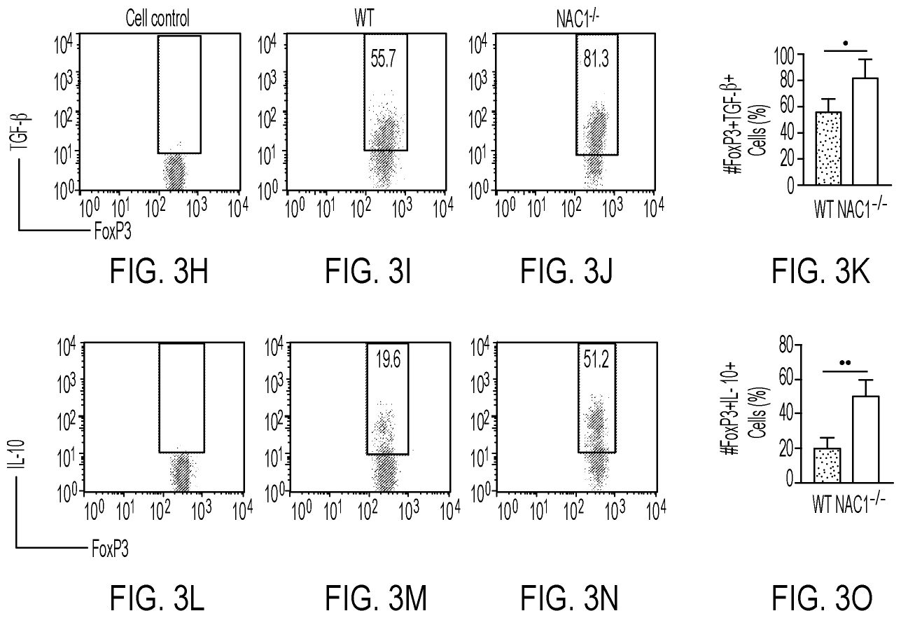

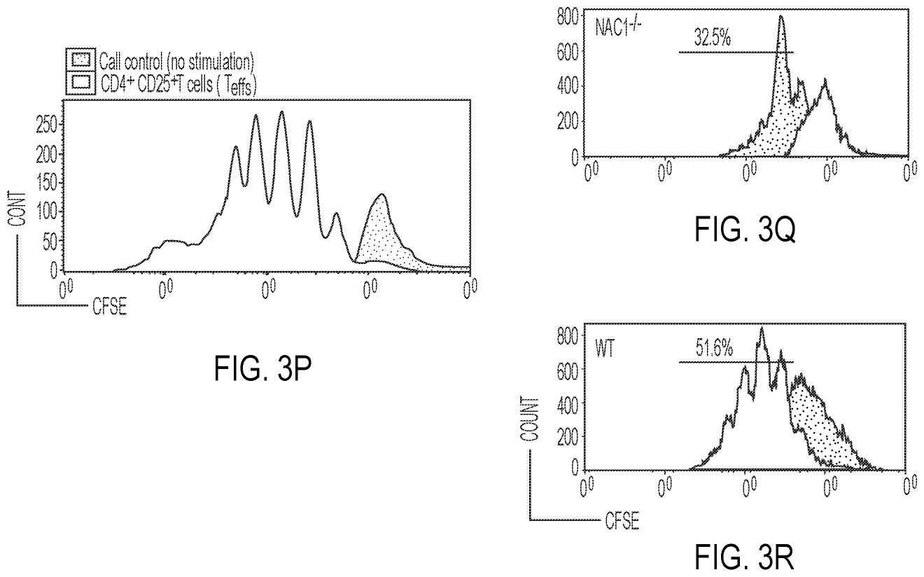

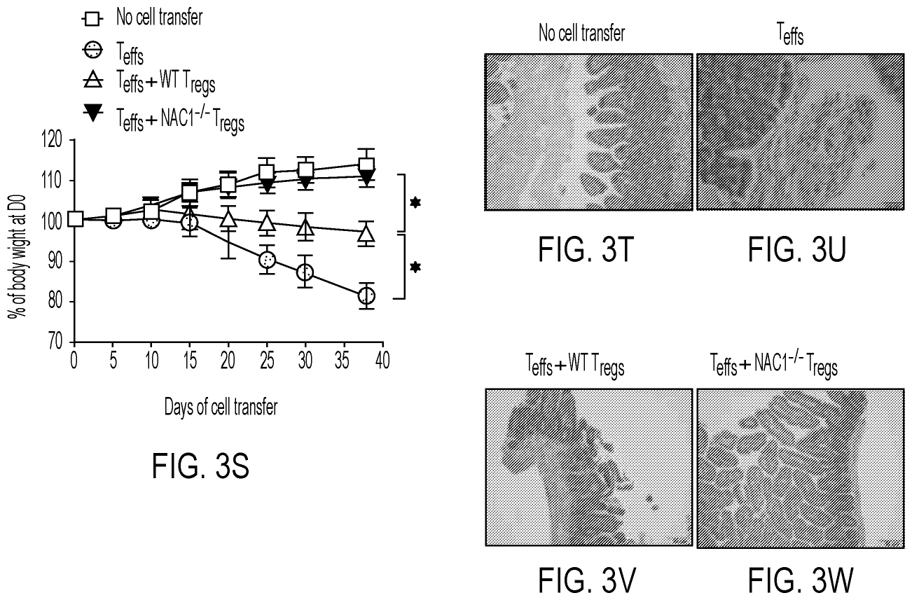

To further validate the negative control of Treg development by NAC1, the functional activity of the Tregs either from WT or NAC1^{-/-} mice was examined. CD4⁺CD25⁺ Tregs from the LNs and spleen of WT or NAC1^{-/-} mice were stimulated with the mouse CD3/CD28-loaded beads in the presence of rIL-2, and the metabolic differences between WT or NAC1^{-/-} cells were then analyzed using the Seahorse XF Cell Mito Stress Test kit. Although Tregs from WT or NAC1^{-/-} mice had similar proliferation and survival profiles (FIGS. 3A-3C), Tregs from NAC1^{-/-} mice exhibited a significantly higher oxygen consumption rate (OCR) and glycolytic rate (glycoPER) than Tregs from WT mice (FIGS. 3D-3G), indicating that NAC1^{-/-} Tregs are metabolically more active than the corresponding control Tregs. Consistently, NAC1^{-/-} Tregs produced significantly greater amounts of the suppressive cytokines, TGF-β and IL-10, than WT Tregs (FIGS. 3H-3O). These results clearly demonstrate a negative impact of NAC1 on the suppressive function of Tregs and inhibiting NAC1 may modulate autoimmunity through promoting Treg development and function. The enhanced function of NAC1^{-/-} Tregs was further demonstrated in an in vitro suppressive assay (FIGS. 3P-3R) and in an autoimmune colitis model subjected to in vivo co-transfer of Tregs with CD4⁺ T effectors (FIGS. 3S-3W), which showed that NAC1^{-/-} Tregs elicited a stronger immune suppressive effect on inflammation than WT Tregs. In addition, in a mouse tumor model receiving the in vivo co-transfer of Tregs with CD8⁺ T cells, NAC1^{-/-} Tregs displayed greater suppressive effect on antitumor immunity than the control Tregs, another evidence for the enhanced suppressive activity of NAC1^{-/-} Tregs.

FIGS. 3A-3W depict the loss of NAC1 enhancing the functional activity of Tregs. Purified CD4⁺ Tregs from the pooled LNs and spleen of WT or NAC1^{-/-} mice were stimulated with anti-CD3 plus CD28 antibodies in the presence of rIL-2 for 6 hr. FIG. 3A is a graphical representation of CD4⁺ Tregs in WT or NAC1^{-/-} mice when analyzed using flow cytometry. FIGS. 3B and 3C are graphical representations of percentages and numbers, respectively, of cell recovery on various days of analyses. The numbers of T cells present on day 0 were assigned a value of 100%, and numbers surviving on various days were used to calculate the percentage recovery relative to day 0. The data shown represent the mean S.E.M. of percentage change of three independent experiments. All P>0.05, Student's unpaired t-test). FIGS. 3D and 3E are graphical representations of the Oxygen consumption rate (OCR, y axis) trace and tabulated data (FIG. 3E) of Tregs. FIGS. 3F and 3G are graphical representations of the glycolytic rate (glycoPER, y axis) trace and tabulated data (FIG. 3G) of Tregs. * P<0.05, ** P<0.01, ****, P<0.0001, Student's unpaired t-test. FIG. 3H, FIG. 3I, and FIG. 3J are flow cytometry analyses of expression of expression of TGF-β in control, WT, and NAC1^{-/-} Tregs, respectively. FIG. 3K is a graphical representation of number of FOXP3⁺ TGF-β⁻ cells in WT or NAC1^{-/-} Tregs when analyzed using flow cytometry. *P<0.05, ** P<0.01, Student's unpaired t-test). FIG. 3L, FIG. 3M, and FIG. 3N are flow cytometry analyses of expression of expression of IL-10 in control, WT, and NAC1^{-/-} Tregs, respectively. FIG. 3O is a graphical representation of number of FOXP3⁺IL-10⁺ cells in WT or NAC1^{-/-} Tregs when analyzed using flow cytometry. * P<0.05, ** P<0.01, Student's unpaired t-test. FIGS. 3P, 3Q, and 3R are graphical representations of results from in vitro suppressive assay. CD4⁺ CD25^{-/-} T effectors pre-labelled with CFSE were stimulated with anti-CD3 plus CD28 antibodies in the absence (FIG. 3P) or presence of WT (FIG. 3Q) or NAC1^{-/-} Tregs (FIG. 3R) (1:1) for three days. Cell proliferation was analyzed by flow cytometry, gating on CFSE⁺ population. FIG. 3S is a graphical representation of changes of body weight of Rag1^{-/-} host mice after adoptive cell transfer of naive CD4⁺ T effectors (Teffs) with or without Tregs from WT or NAC1^{-/-} mice (n=5; *, p<0.05, Nested t test). FIGS. 3T, 3U, 3V and 3W are representative photographic images of H&E-stained sections of cecum tissues collected from control mice (FIG. 3T) and the Rag1^{-/-} host mice after adoptive cell transfer of naive CD4⁺ T effectors (Teffs) (FIG. 3U) and with or without Tregs from WT or NAC1^{-/-} mice (FIG. 3V and FIG. 3W, respectively).

### NAC1-Deficient Mice are Insusceptible to Induction of Autoimmunity

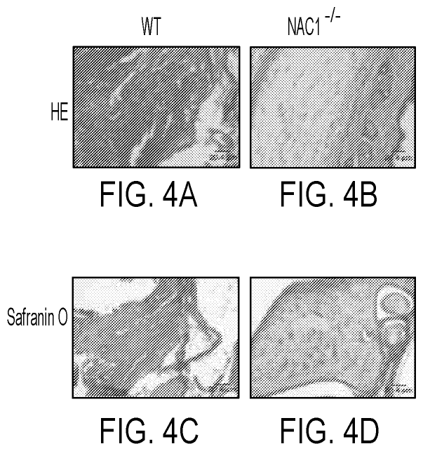

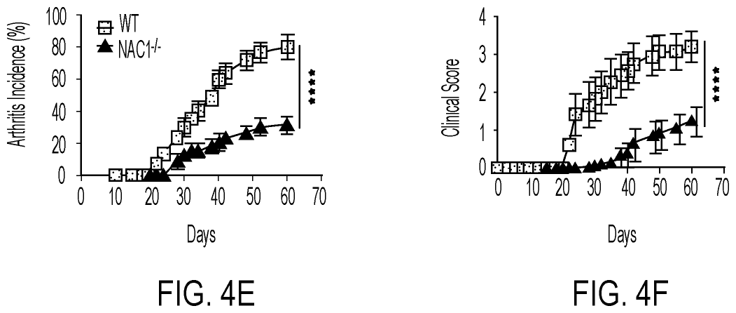

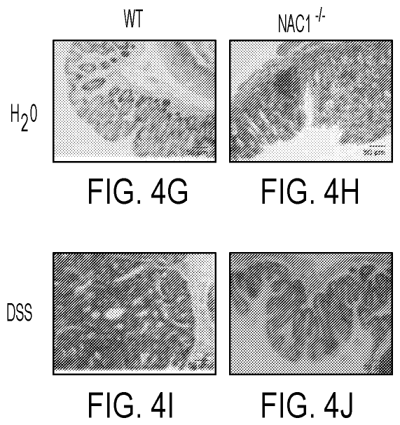

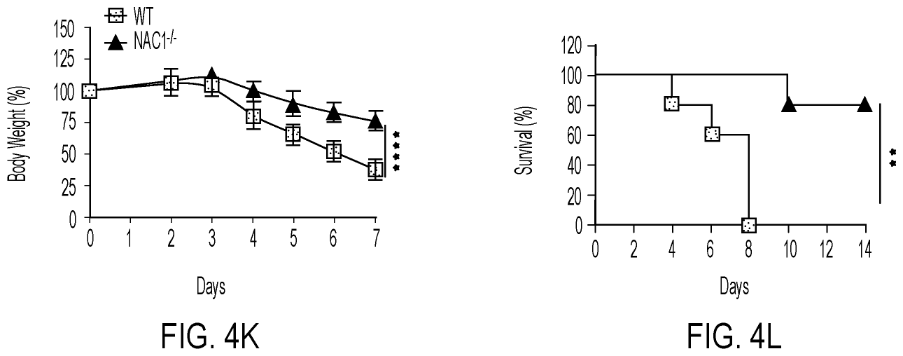

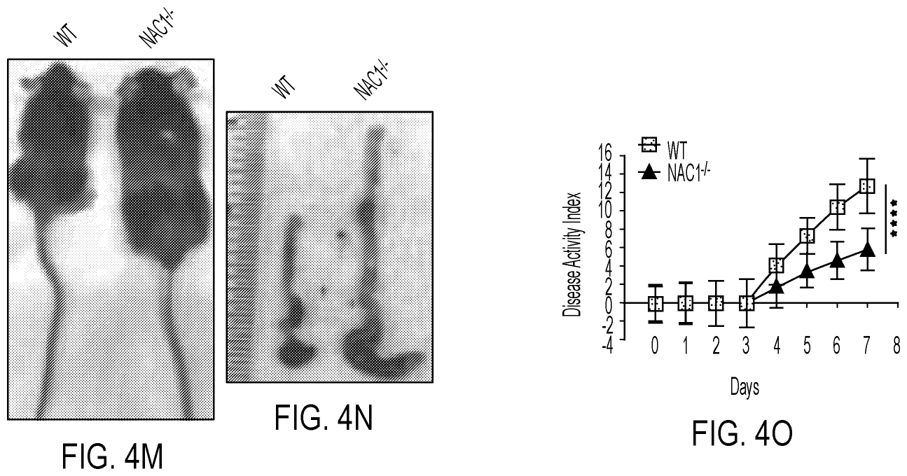

To further prove the impact of NAC1 on autoimmunity, the response of the WT and NAC1^{-/-} mice to induction of autoimmune arthritis and colitis were compared. Type II collagen was used to induce arthritis and dextran sulfate sodium (DSS) was given to mice to induce colitis. NAC1-deficient (NAC1^{-/-}) mice were significantly tolerant to induction of autoimmune arthritis and colitis (FIGS. 4A-4O). In the collagen-induced arthritis model (CIA), a significantly lower occurrence of CIA was observed in NAC1^{-/-} mice than in the littermate controls, as determined by the histologic evidence (FIGS. 4A-4D), disease incidence (FIG. 4E; p<0.0001) and disease score (FIG. 4F; p<0.0001). Tolerance to autoimmunity induction was recapitulated in a colitis model in which mice were given drinking water containing dextran sulfate sodium (DSS). WT mice developed autoimmune colitis within 3-4 days following DSS administration and showed visible signs of illness including hunched back, raised fur, symptoms of sepsis and reduced mobility because of diarrhea and anemia; strikingly, the occurrence of colitis declined remarkably in NAC1^{-/-} mice (FIGS. 4D-4O). In NAC1^{-/-} mice, the body weight loss (FIG. 4K), survival (FIG. 4L), colon shrinkage (FIG. 4N) and the disease activity index (FIG. 4O) were all significantly improved as compared with WT animals (p<0.0001). In both disease models, there were considerably larger amounts of pro-inflammatory immune cells in the joint (FIGS. 4A-4D) and the gut (FIGS. 4G-4J) tissues of WT mice than in those of NAC1^{-/-} animals, suggesting that weakened immune response accounts for the insusceptibility of NAC1^{-/-} mice to induction of autoimmunity. As NAC1^{-/-} mice showed enhanced amounts and functions of Tregs (FIGS. 1A-1O and FIGS. 3A-3W) and Tregs have a unique capacity to suppress immune response, the tolerance to the induction of autoimmune diseases observed in NAC1^{-/-} mice (FIGS. 4A-4O) could be a consequence of the enhanced Treg stability.

FIGS. 4A-4O demonstrate that NAC1^{-/-} mice are tolerant to induction of autoimmunity. WT or NAC1^{-/-} mice were challenged with either bovine type II collagen in complete Freund's adjuvant by one intradermal immunization at two sites in the base and slightly above of the tail on day 0, or by oral ingestion of 3% dextran sulfate sodium (DSS, MP Biomedicals) in drinking water for 5 days. FIGS. 4A-4D are photographic images of hematoxylin and safranin O staining of WT mice (FIGS. 4A and 4C) or NAC1^{-/-} mice (FIGS. 4B and 4D) to show the histology of the joints in an arthritic model. FIGS. 4E and 4F are graphical representations of arthritis incidence (FIG. 4E), and clinical score (FIG. 4F) as evaluated by examining the paws. Values are the mean S.E.M. of three independent experiments (n=10). P<0.0001 in B and C, simple linear regression. FIGS. 4G-4J are photographic images of the histology of the colon in H₂O and DSS-challenged WT mice (FIGS. 4G and 4I) and H₂O and DSS-challenged NAC1^{-/-} mice (FIGS. 4H and 4J) to show a colitis model. The severity of colitis activity was graded on the designated dates. FIGS. 4K and 4L are graphical representations of animal body weight change (FIG. 4K) and survival (FIG. 4L) of the WT or NAC1^{-/-} mice. FIGS. 4M and 4N are photographic images of the WT or NAC1^{-/-} animals and their colon length, respectively. FIG. 4O is a graphical representation of the resultant IBD disease activity index. Values are the mean±S.E.M. of three independent experiments (n=10). P<0.0001 in FIGS. 4K and 4O, simple linear regression. P<0.001 in FIG. 4L, survival curve comparison.

Autoimmune diseases such as type 1 diabetes, rheumatoid arthritis, ulcerative colitis and Crohn's disease are presumed to result from interaction between genetic and environmental factors and to be a consequence of compromised immune tolerance versus adaptive immune response; yet, how impaired balance between immune response and tolerance is triggered as well as the mechanisms by which tolerance is established and maintained remain elusive. How the stability of FoxP3 and suppressor Tregs are regulated is an important theme in Treg biology. NAC1 was identified as a critical determinant of immune tolerance. NAC1^{-/-} mice are substantially tolerant to the induction of autoimmunity, as evidenced by the significantly decreased occurrences of autoimmune arthritis and colitis (FIGS. 4A-4O). The promotive effect of NAC1 on autoimmunity is mediated through its negative regulation of the stability of Treg and FoxP3.

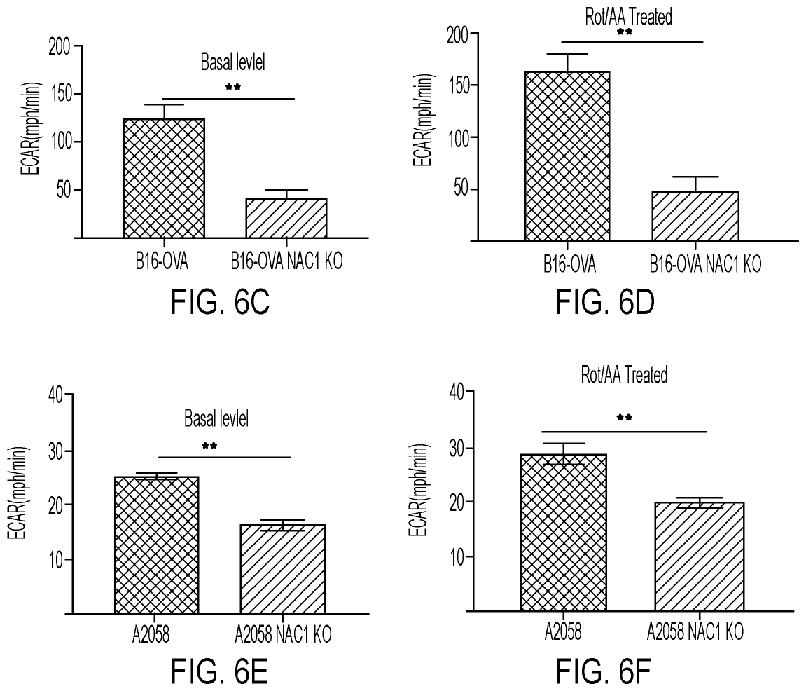

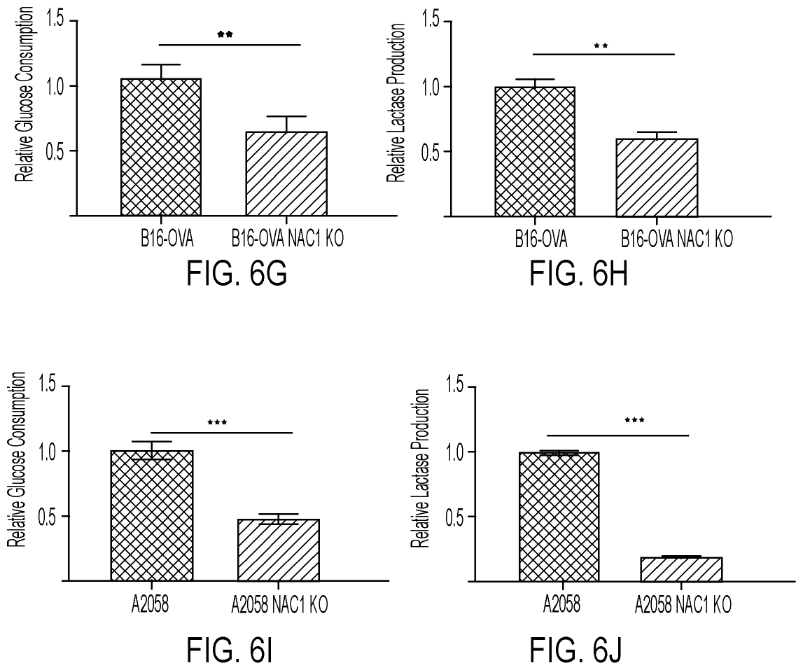

Although DNA methylation of FoxP3 has been reported to be associated with the stability and function of Tregs, the effects of NAC1 on Tregs do not appear to be associated with alterations in DNA methylation of FoxP3. The DNA methylation of FoxP3 in WT and NAC1^{-/-} Tregs was compared and the DNA methylation of FoxP3 was examined in the Treg-specific demethylated region (TSDR) of CNS2 (ADS443) and FoxP3 proximal promoter region (ADS1183). As a control, FoxP3 DNA methylation was similar in the unsorted lymphocytes from the LNs and spleen of NAC1^{-/-} mice (Table 1). In these experiments, greater than 100 CpG sites in the FoxP3 DNA promoter regions showed that there was no significant difference between WT and NAC1^{-/-} Tregs, indicating that NAC1 does not affect the DNA methylation of FoxP3. Moreover, we found that NAC1 does not act as a transcriptional regulator (FIG. 6). Instead, we show that NAC1 can interfere the acetylation and degradation of FoxP3 protein. Thus, the role of NAC1 in the post-transcriptional regulation of FoxP3 may account for the upregulation of FoxP3 in NAC1^{-/-} Tregs.

Treg stability is vital to the maintenance of immune tolerance but is often altered in autoimmunity; yet, how destabilization of Tregs occurs in autoimmune diseases remains elusive. Data here demonstrate that that may be the result of concomitant upregulation of NAC1 and downregulation of FoxP3 in Tregs treated with the pro-inflammatory cytokines such as IL-10 and TNF-α. The “basal” level of NAC1 in Tregs plays an important role in leashing the immune tolerance to keep the immune system vigilant to pathogens; inflammatory stimulation induces upregulation of NAC1, and this in turn destabilizes FoxP3 and converts FoxP3⁺ Tregs to FoxP3-Tregs that then become Th1 or Th17 CD4⁺ T effector cells, further breaking tolerance and instigating strong immune response.

### NAC1 and Cancer

Expression of NAC1 is closely associated with tumor development and poor prognosis in various types of cancers such as melanoma, urethral, ovarian, and lung cancer. NAC1 promotes autophagic response, disables cellular senescence, and facilitates oxidative stress resistance during cancer progression. More recently, NAC1 is a positive regulator of glycolysis in ovarian cancer through its stabilization of HIF-1α. The upregulation of glycolysis by tumor cells may contribute substantially to an acidic TME that suppresses the antitumor immune response and promotes cancer development.

NAC1 expression in melanoma cells contributes substantially to immune evasion through its positive regulation of lactate dehydrogenase A (LDHA) expression, leading to increased lactic acid (LA) production. Depleting tumor NAC1 can effectively suppress LDHA transcription, enhance the functional status of cytotoxic CD8⁺ T cells and reinforce the efficacy of ACT against melanoma.

Expression of NAC1 is Associated with the Prognosis of Melanoma Patients and Proliferation of Tumor Cells.

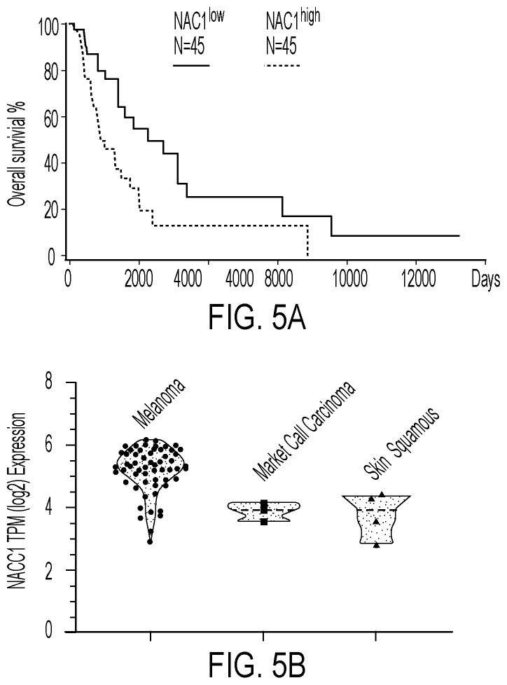

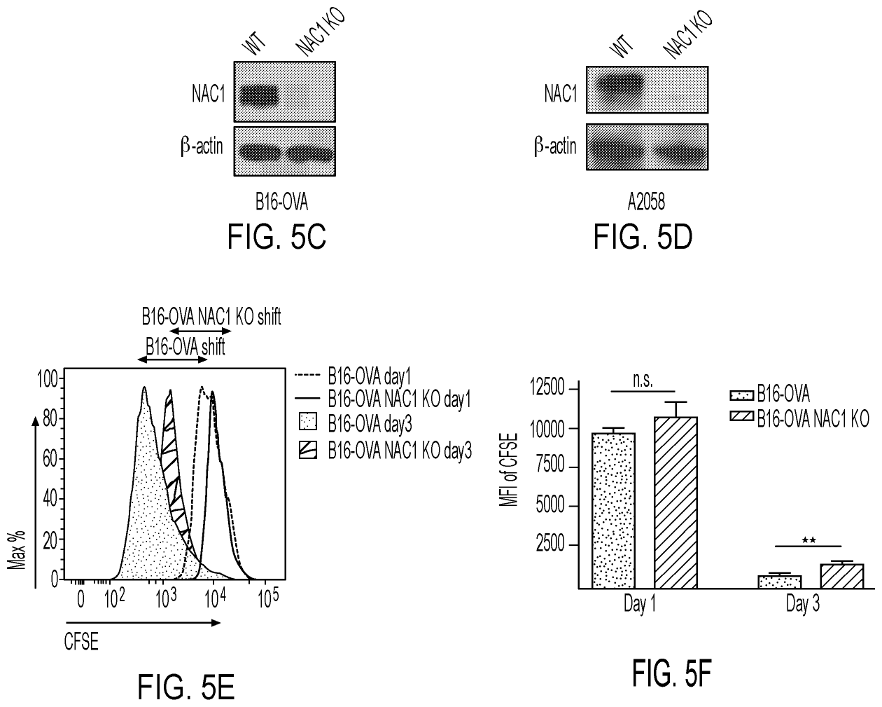

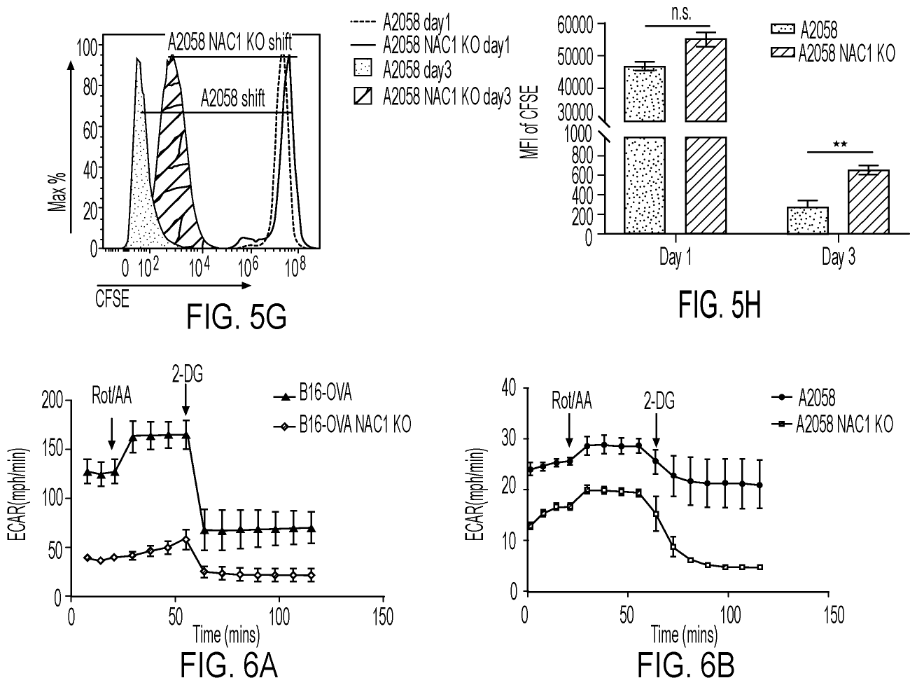

Analysis of TCGA dataset showed that patients with high NAC1 expression had a greatly reduced OS time as compared with the patients with low NAC1 expression (FIG. 5A). NAC1 expression was higher in melanomas than in Merkel cell and skin squamous carcinomas (FIG. 5B). NAC1 was knocked out in mouse B16 melanoma cells (FIG. 5C) and human A2058 melanoma cells (FIG. 5D) by Western blots. NAC1 KO in tumor cells remarkably inhibited their proliferation, as assayed by cell counting and cell division (FIGS. 5E-5H). These results suggest a role of NAC1 in tumor development and progression.

FIGS. 5A-5H demonstrate the effects of NAC1 on proliferation of melanoma cells and development of melanoma. FIG. 5A is a graphical representation of the survival of melanoma patients with high expression (top 30%; red) and low expression of NAC1 (bottom 30%; blue), calculated with the ‘R2: Tumor Skin Cutaneous Melanoma-TCGA-470-rsem-tcgars’ dataset (http://r2.amc.nl) from the TCGA database. FIG. 5B is a graphical representation of the distribution of NAC1 expression in human skin cancer cell lines (melanoma, Merkel cell carcinoma, and skin squamous carcinoma), determined by the CCLE database (https://portals.broadinstitute.org/ccle). FIG. 5C is a photographical representation of the Western blot analysis of protein expression of NAC1 and β-actin in WT and NAC1-deficient (NAC1 KO) mouse B16-OVA melanoma cells. FIG. 5D is a photographical representation of the Western blot analysis of protein expression of NAC1 and β-actin in WT and NAC1 KO human A2058 melanoma cells. FIG. 5E and FIG. 5F are graphical representations of the cell division rates of WT and NAC1 KO mouse B16-OVA melanoma cells as analyzed by CFSE between day 1 and day 3. FIG. 5G and FIG. 5H are graphical representations of the cell division rates of WT and NAC1 KO human A2058 melanoma cells were analyzed by CFSE between day 1 and day 3. In these experiments, the tumor cells were labeled with CFSE dye (5 μM) prior to seeding onto the 48-well tissue culture plates. The CFSE intensities were analyzed by flow cytometer (FIG. 5E and FIG. 5G) and quantified (FIG. 5F and FIG. 511) by the mean fluorescence intensity (MFI) of CFSE. One representative of three identical experiments is shown. **p<0.01. CCLE, cancer cell line encyclopedia; CFSE, carboxyfluorescein succinimidyl ester; NAC1, nucleus accumbens-associated protein-1; n.s., not significant; OVA, ovalbumin; TCGA, The Cancer Genome Atlas.

### NAC1 Supports Glycolysis of Melanoma Cells.

NAC1 plays a critical role in promoting glycolysis in hypoxic ovarian cancer cells. Consistently, melanoma cells with depletion of NAC1 showed a decreased ECAR when compared with the control cells (FIGS. 6A-6B). Furthermore, the basal level (FIGS. 6C-6E) and the maximum level (FIGS. 6D-6F) of ECAR were significantly lower in NAC1 KO B16-OVA cells and NAC1 KO A2058 cells when compared with B16-OVA and A2058 WT control cells. Treatment of the melanoma cells with rotenone and antimycin A (Rot/AA), inhibitors of the oxidative phosphorylation pathway, caused an elevated ECAR level in the WT control cells. However, the NAC1 KO tumor cells could not raise the ECAR level due to insufficient glycolysis support. Moreover, the lactate production and glucose uptake in the cellular supernatants of these melanoma cells was reduced (FIGS. 6G-6J). As expected, mouse NAC1 KO B16-OVA cells and human NAC1 KO A2058 cells secreted significantly less lactate compared with WT cells and altered glucose consumption compared with WT cells in vitro. Although OVA is not a natural Ag of mouse tumors, here B16-OVA cells were used as a model for cancer immunotherapy because expression of OVA can facilitate strong immune responses to tumor Ags. Thus, NAC1 plays a critical role in supporting metabolic reprogramming in melanoma cells.

FIGS. 6A-6J are graphical representations to demonstrate depleting NAC1 decreases the glycolytic rate of melanoma cells. FIG. 6A is a graphical representation of the time series of ECAR measurements in WT and NAC1 KO B16-OVA cells by the Seahorse Metabolic Analyzer. FIG. 6B is a graphical representation of the time series of ECAR measurements in WT and NAC1 KO A2058 cells by the Seahorse Metabolic Analyzer. Data are represented as mean±SEM; n=6 wells per condition from two independent experiments. FIG. 6C is a graphical representation of the basal glycolysis rates of WT and NAC1 KO B16-OVA cells. FIG. 6D is a graphical representation of the maximum glycolysis rates of WT and NAC1 KO B16-OVA cells. FIG. 6E is a graphical representation of the basal glycolysis rates of WT and NAC1 KO A2058 cells. FIG. 6F is a graphical representation of the maximum glycolysis rates of WT and NAC1 KO A2058 cells. In FIGS. 6C-6F, six wells per condition from two independent experiments were used and a one-way ANOVA with multiple comparisons correction was performed. FIG. 6G is a graphical representation of the quantification of relative glucose consumption in WT and NAC1 KO B16− OVA cells. FIG. 6H is a graphical representation of quantification of lactase production in the supernatant of WT and NAC1 KO B16-OVA cells. FIG. 6I is a graphical representation of the quantification of relative glucose consumption in WT and NAC1 KO A2058 cells. FIG. 6J is a graphical representation of the quantification of lactase production in the supernatant of WT and NAC1 KO A2058 cells. **p≤0.01, ***p≤0.001. ANOVA, analysis of variance; ECAR, extracellular acidification rate; NAC1, nucleus accumbens-associated protein-1; OVA, ovalbumin. Depletion of tumorous NA C1 invigorates cytotoxic CD8⁺ T cells.

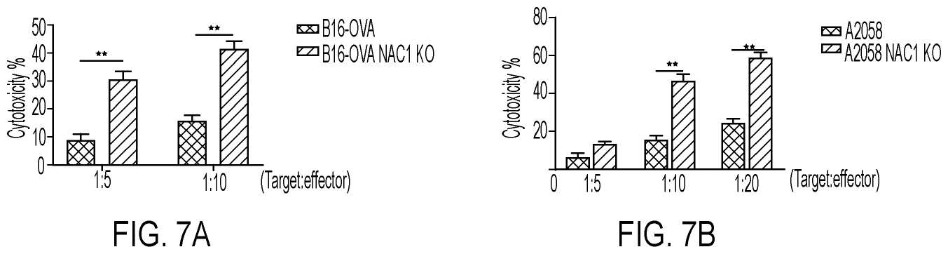

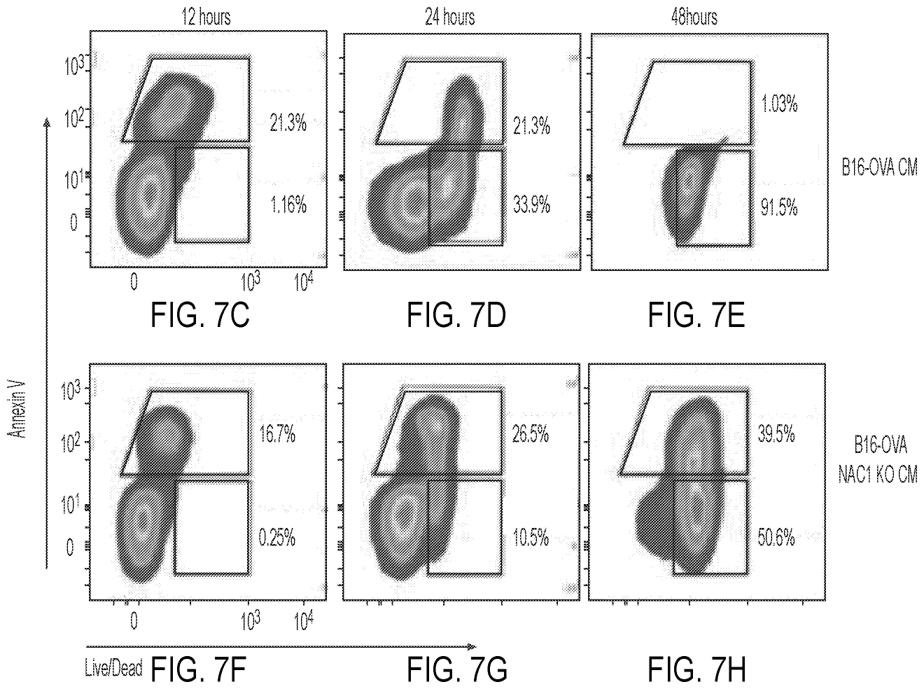

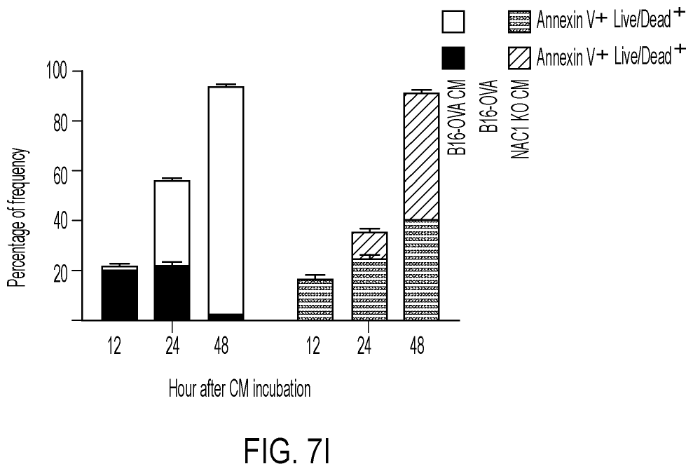

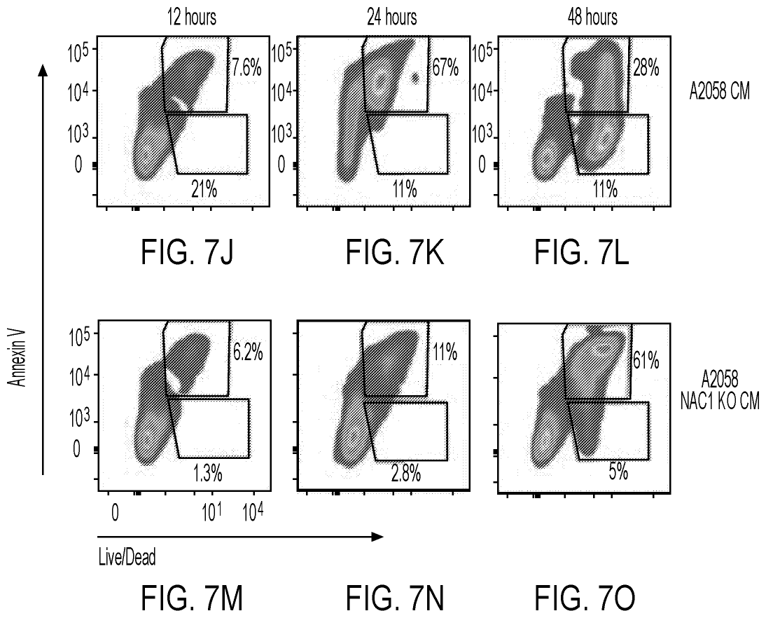

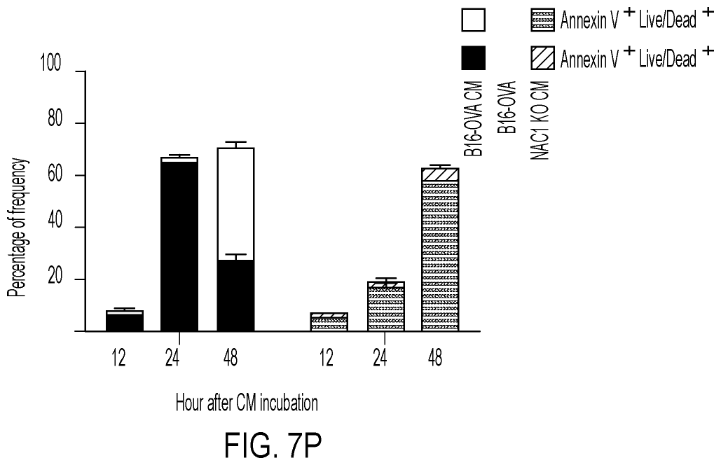

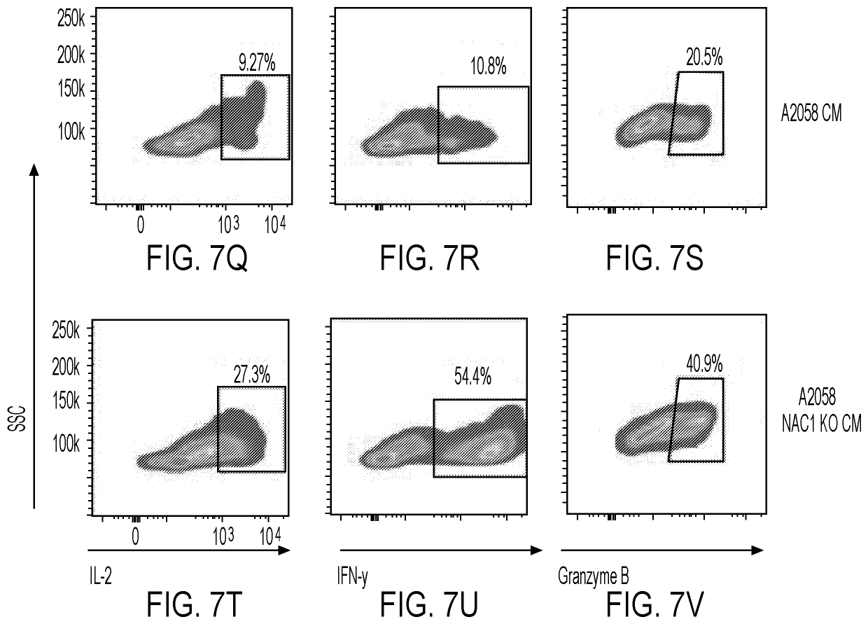

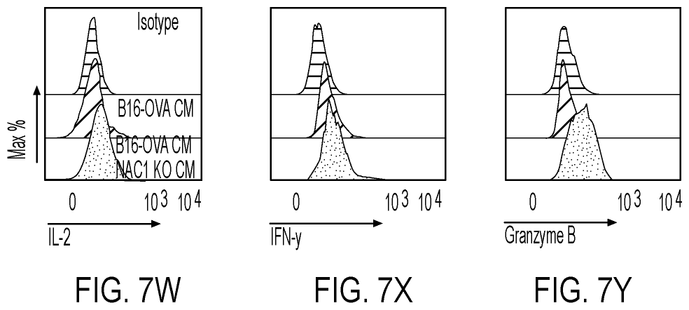

The activity of immune cells can be impacted by the metabolic alteration of tumor cells. Tumor cells have high glycolytic activity, leading to their secretion and accumulation of lactate and subsequent development of an acidic TME. The TME affects the development and function of immune cells through numerous avenues. For instance, massive LA production from tumor cells inhibit T cell cytotoxicity and effector functions. Expression of NAC1 negatively regulates the suppressive activity of regulatory T cells (T_{reg}). Therefore, it was evaluated whether the tumorous expression of NAC1 affects the cytocidal effect/activity of CD8⁺ T cells. The WT or NAC1 KO B16-OVA cells were co-cultured with CD8⁺ T cells prepared from the OT-I T cell receptor (TCR) transgenic mice, which specifically recognize ovalbumin (OVA) present on B16-OVA cells. The cytotoxicity of CD8⁺ T cells against tumor cells was significantly increased in NAC1 KO B16− OVA cells compared with that in WT cells (FIG. 7A). To further validate the effects of NAC1 on the cytotoxicity of human CD8⁺ T cells, human A2058 melanoma cells were used, which expressed tyrosinase Ag, and tyrosinase-specific human CD8⁺ T cells was constructed by transduction with a retroviral vector of anti-tyrosinase TCR. Similar results were obtained from the co-culture of human WT or NAC1 KO A2058 melanoma cells with the tyrosinase-specific human CD8⁺ T cells (FIG. 7B). To explore how expression of NAC1 in tumor cells alters the cytocidal effect of CD8⁺ T cells, we determined the effect of the CM from the cultures of WT B16-OVA, NAC1 KO B16− OVA, WT A2058, or NAC1 KO A2058 cells on CD8⁺ T cells. In these experiments, activated CD8⁺ T cells were incubated in the CM for 12, 24, or 48 hours. The incubation period was followed by assessments of CD8⁺ T cell apoptosis and function. Apoptosis of CD8⁺ T cells cultured in NAC1 KO B16-OVA or A2058 CM was significantly reduced compared with those cultured in the WT CM (FIGS. 7C-7P). Moreover, CD8⁺ T cells cultured in the NAC1 KO tumor CM produced more significant amounts of cytokines IFN-γ, granzyme B, and IL-2, than those cultured in the media of control tumor cells (FIGS. 7Q-7Y). This indicates that the expression of NAC1 in tumors can impair the activity of CD8⁺ T cells. In addition, CD8⁺ T cells cultured in the media of WT tumor cells had a higher percentage of PD-1⁺ TIM-3⁺ than those cultured in the NAC1 KO tumor CM, indicating that tumorous NAC1 may induce exhaustion of CD8⁺ T cells.

FIGS. 7A-7Y demonstrate that depletion of tumorous NAC1 strengthens the cytotoxicity of CD8⁺ T cells. For FIG. 7A, WT or NAC1 KO B16-OVA cells (targets) were co-cultured with OT-I CD8⁺ T cells (effectors) (ratio: 1:5 and 1:10) for 6 hours. FIG. 7A is a graphical representation of the quantification of percent cytotoxicity when calculated as (1−(R5/R0))×100, where R5=(target cells as % of total of 6 hours)/(effector cells as % of total of 6 hours), R0=(target cells as % of total of 0 h)/(effector cells as % of total at 0 h). For FIG. 7B, WT or NAC1 KO A2058 cells were co-cultured with anti-tyrosinase TCR transduced human CD8⁺ T cells (effectors) (ratio: 1:5, 1:10, 1:20) for 6 hours. FIG. 7B is a graphical representation of the quantification of percent cytotoxicity was calculated as (1−(R5/R0))×100, where R5=(target cells as % of total at 6 hours)/(effector cells as % of total of 6 hours), R0=(target cells as % of total of 0 hour)/(effector cells as % of total of 0 hour). FIGS. 7C-7H are representative contour plots of Annexin V and Live-Dead expression after incubation with WT conditional medium (CM) or NAC1 KO B16-OVA CM for 12 hours (FIG. 7C and FIG. 7F), for 24 hours (FIG. 7D and FIG. 7G), and for 48 hours (FIG. 7E and FIG. 7H). FIG. 71 is a graphical representation of the frequencies of the indicated Annexin V⁺ and Live-Dead⁺ expressing populations after incubation with WT conditional medium (CM) or NAC1 KO B16-OVA CM for 12, 24, and 48 hours. Apoptotic rates were assessed by flow cytometry. One representative experiment out of three is shown. FIGS. 7J-70 are representative contour plots of Annexin V and Live-Dead expression on the CM-treated human CD8⁺ T cells after incubation with WT CM or NAC1 KO A2058 CM for 12 hours (FIG. 7J and FIG. 7M), for 24 hours (FIG. 7K and FIG. 7N), and for 48 hours (FIG. 7L and FIG. 7O). FIG. 7P is a graphical representation of the frequencies of the indicated Annexin V⁺ and Live-Dead⁺ expressing populations after incubation with WT CM or NAC1 KO A2058 CM for 12, 24, and 48 hours, respectively. Apoptotic rates were assessed by flow cytometry. One representative experiment out of three is shown. FIGS. 7Q-7V are histograms showing expression of the indicated cytokines after incubation with WT CM and NAC1 KO A2058 CM for 6 hours-IL-2 (FIG. 7Q and FIG. 7T), IFN-γ (FIG. 7R and FIG. 7U), and Granzyme B (FIG. 7S and FIG. 7V), respectively. FIGS. 7Q-7V are histograms showing expression of the indicated cytokines after incubation with WT CM or NAC1 KO B16-OVA CM for 6 hours-IL-2 (FIG. 7W), IFN-γ (FIG. 7X), and Granzyme B (FIG. 7Y), respectively. One representative of three identical experiments is shown. IFN-γ, Granzyme B, and IL-2 expression were determined by flow cytometry. **p≤0.01; one way analysis of variance with multiple comparison correction. NAC1, nucleus accumbens-associated protein-1; OVA, ovalbumin.

### NAC1-Mediated LDHA Expression Contributes to LA Production.

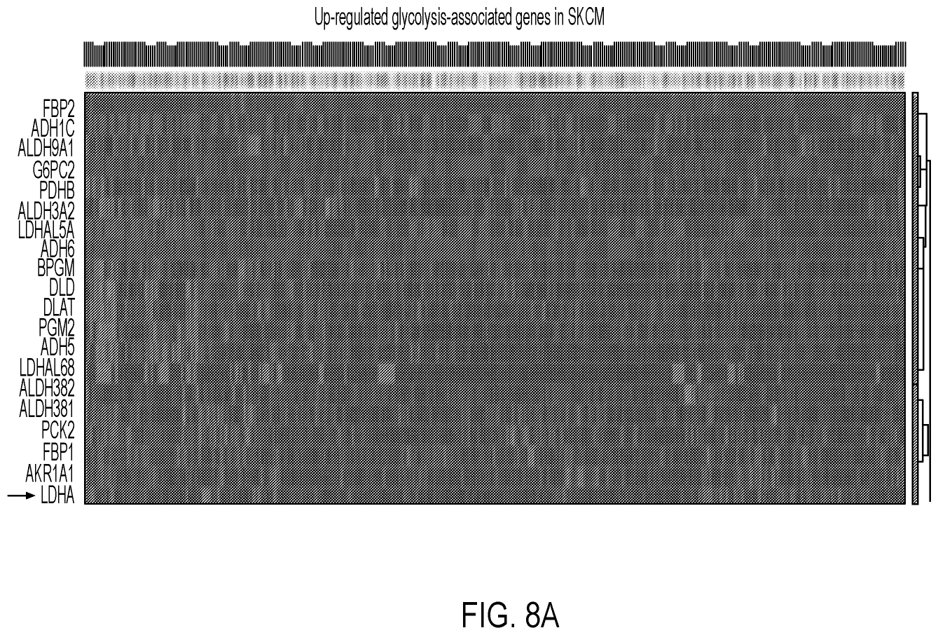

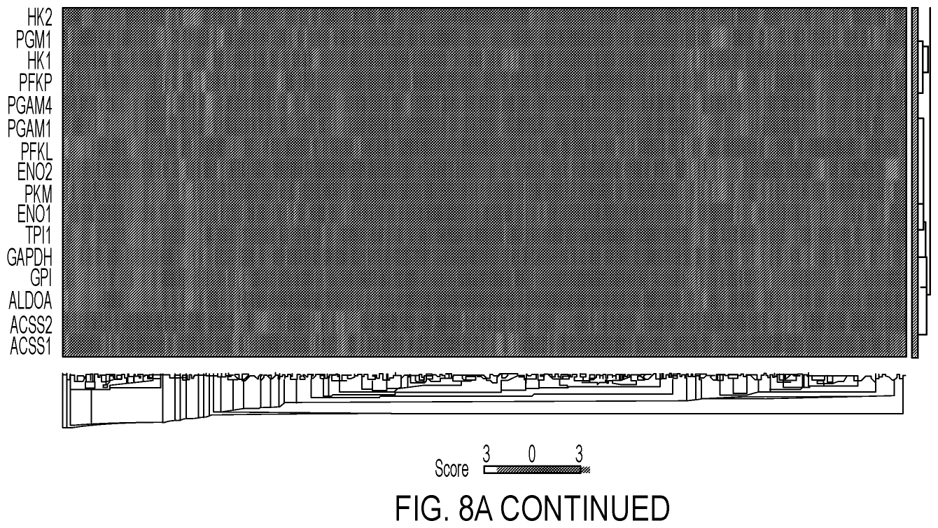

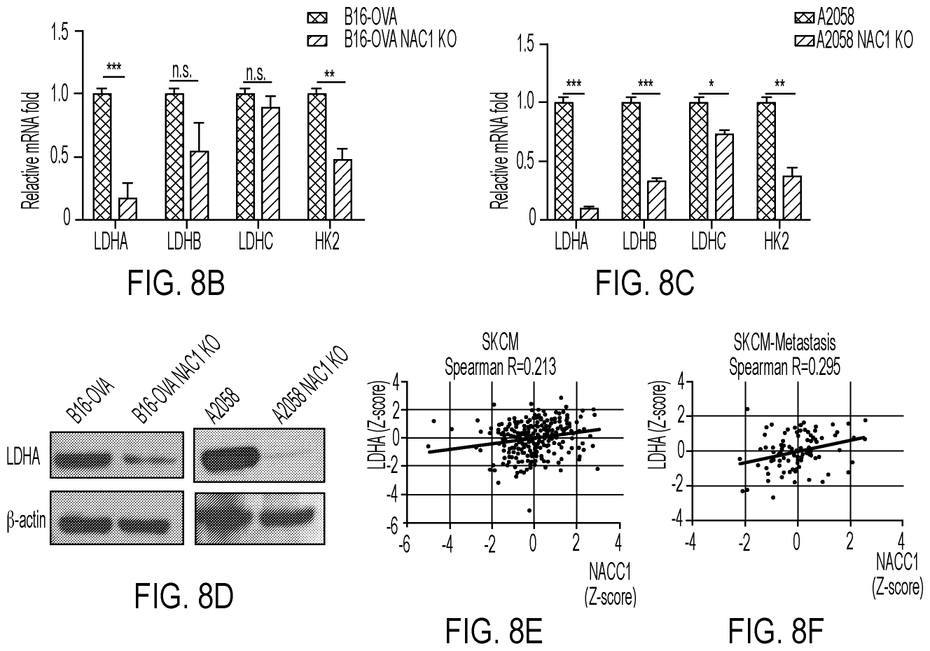

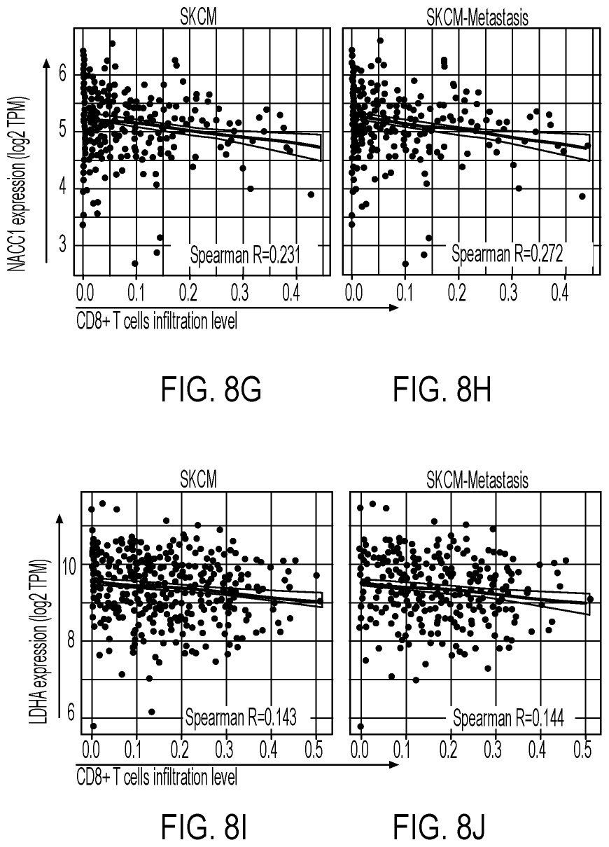

Because NAC1 has a critical role promoting glycolysis in melanoma cells (FIG. 6), the impact of tumorous NAC1 was evaluated on the glycolytic activity of CD8⁺ T cells. Using the TCGA-melanoma database, the possible association between NACC1 and glycolysis related genes were analyzed (FIG. 8A). Among the positively correlated genes, expression of LDHA demonstrated a strong correlation with NAC1 expression, consistent with ovarian cancer models. LDH family genes are the primary metabolic enzymes that convert pyruvate to lactate and vice versa. In addition, the enzymes also play a significant role in regulating nutrient exchange between tumor and stroma. To determine how NAC1 regulates the expression of LDHA, NAC1's effects on the mRNA expression of LDHA and other LDH family genes in the tumor cells were evaluated. Using qPCR, the LDHA and hexokinase 2 (HK2) mRNA levels were found to be significantly lower in NAC1 KO B16-OVA and NAC1 KO A2058 tumor cells than in B16-OVA or A2058 cells (FIGS. 8B-8C). This indicates NAC1 plays a role in the regulation of LDHA transcription. To confirm this observation in melanoma cells, the protein expression of LDHA in NAC1-expressing and NAC1 KO tumor cells was compared through Western blots. LDHA protein was remarkably downregulated in NAC1 KO B16-OVA and A2058 cells, compared with that in WT cells (FIG. 8D). In addition, analyses of the TCGA database revealed that LDHA expression is significantly correlated with NAC1 expression, with a higher correlation rate in patients with poor prognosis (Stage>III) (FIGS. 8E-8F). Using the TIMER, an algorithm was developed to analyze the abundance of tumor-infiltrating immune cells comprehensively. The NAC1-LDHA axis was evaluated for its involvement in the tumor-immune interactions in melanoma patients. The expressions of NACC1 (encoding NAC1) and LDHA (encoding LDHA) are inversely correlated with cytotoxic T lymphocyte (CTL) infiltration in SKCM and SKCM-Metastasis (FIGS. 8G-8J).

FIGS. 8A-8J demonstrate NAC1 promotes the expression of LDHA in tumor cells. FIG. 8A is a heatmap of normalized RNA-seq reads (Z score), top upregulated glycolysis-associated genes in the TCGA SKCM database. FIG. 8B is a graphical representation of the qRT-PCR analysis of mRNA of LDHA, LDHB, LDHC, and HK2 in WT and NAC1 KO B16-OVA cells. FIG. 8C is a graphical representation of the qRT-PCR analysis of mRNA of LDHA, LDHB, LDHC, and HK2 in WT and NAC1 KO A2058 cells. The results are presented relative to the level of GAPDH. n=5 per condition from two independent experiments. *p≤0.05, **p≤0.01, ***p≤0.001. FIG. 8D is a photographical representation of the Western blot analyses of expression of LDHA in whole-cell extracts from WT and NAC1 KO B16-OVA cells (left) and WT and NAC1 KO A2058 cells (right). β-actin was used as a loading control. One representative experiment out of three is shown. FIG. 8E and FIG. 8F are graphical representations of the correlations of NACC1 with LDHA gene expression (Z-score) as determined in a dataset including 470 melanoma tumors and 368 melanoma-metastatic tumors, respectively (‘R2: Tumor Skin Cutaneous Melanoma-TCGA- 470-rsem-tcgars’). Pearson's correlation was calculated. FIG. 8G and FIG. 8H are graphical representations of the correlations of CD8⁺ T cell infiltration with NACC1 expression in melanoma (SKCM, n=470) and metastasis melanoma (SKCM-metastasis, n=368). FIG. 8I and FIG. 8J are graphical representations of the correlations of CD8⁺ T cell infiltration level with LDHA expression in melanoma (SKCM, n=470) and metastasis melanoma (SKCM-metastasis, n=368). All correlations were estimated using TIMER algorithm. Pearson's correlation was calculated. GAPDH, glyceraldehyde 3-phosphate dehydrogenase; LDHA, lactate dehydrogenase A; NAC1, nucleus accumbens-associated protein-1; SKCM, skin cutaneous melanoma; TCGA, The Cancer Genome Atlas; TIMER, Tumor Immune Estimation Resource.

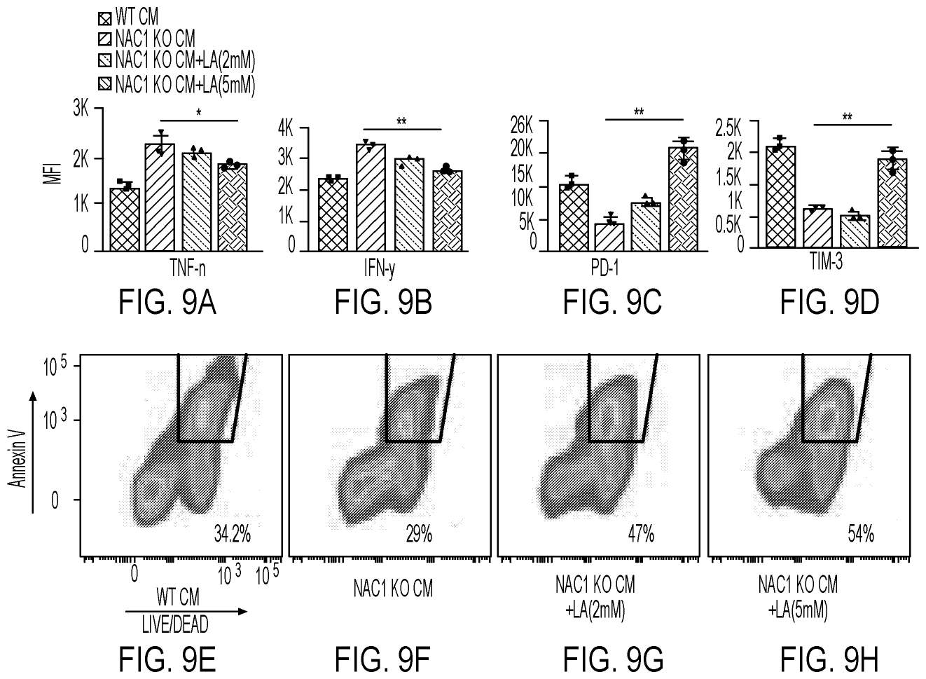

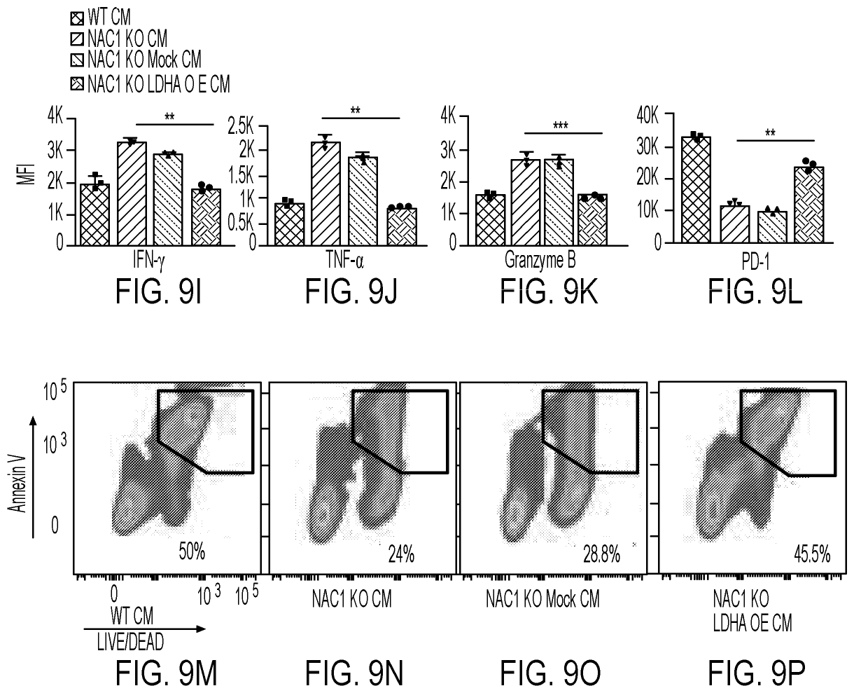

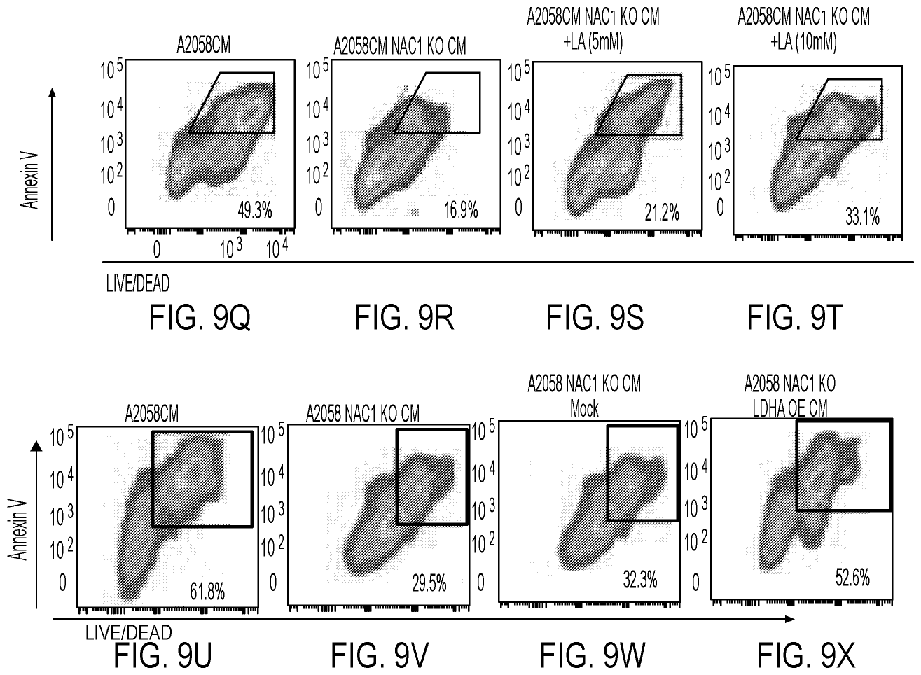

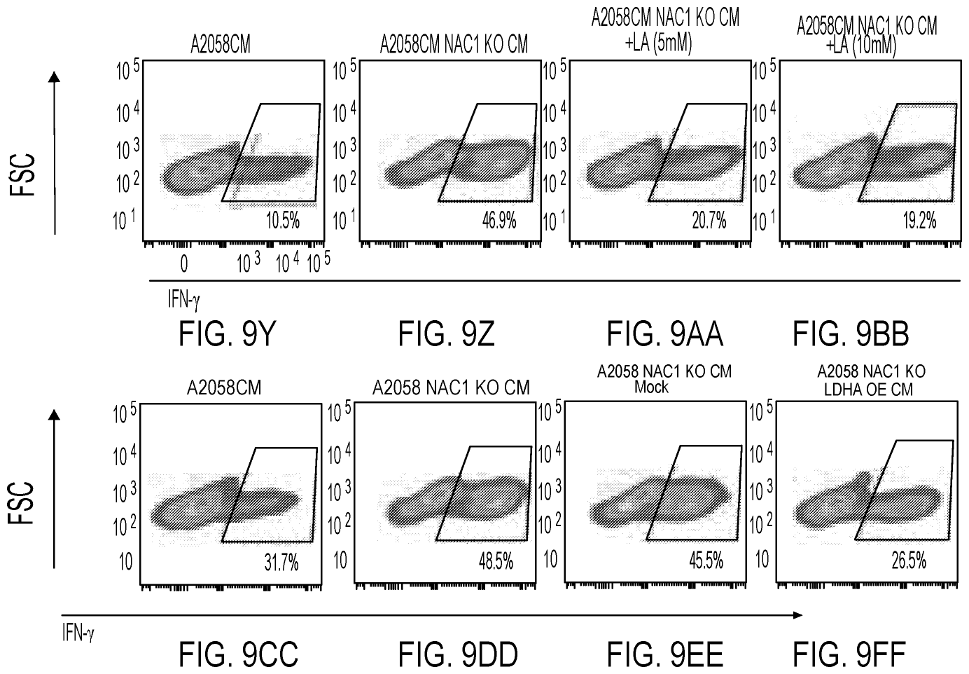

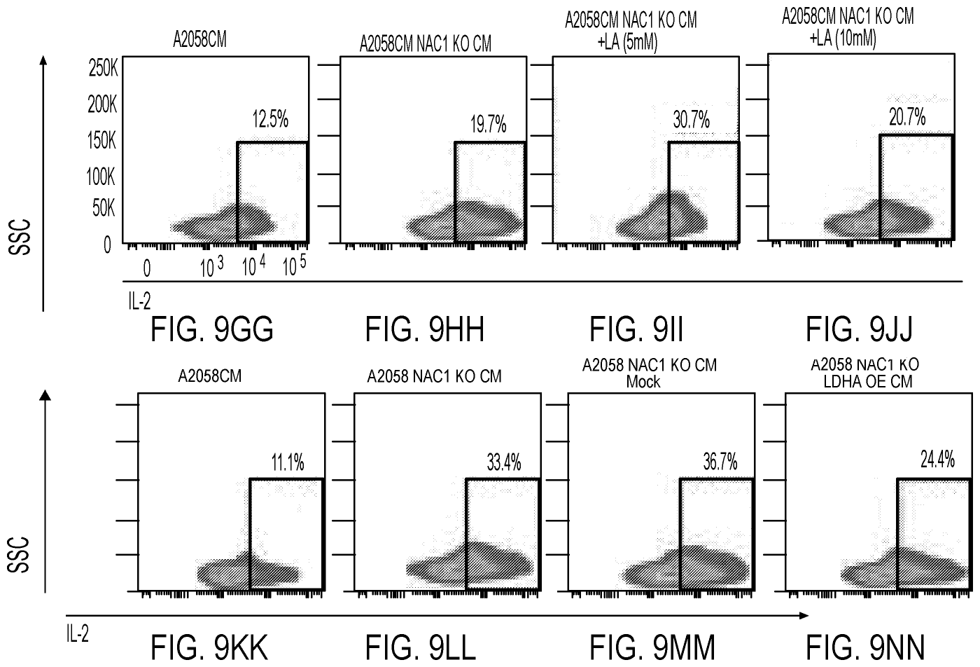

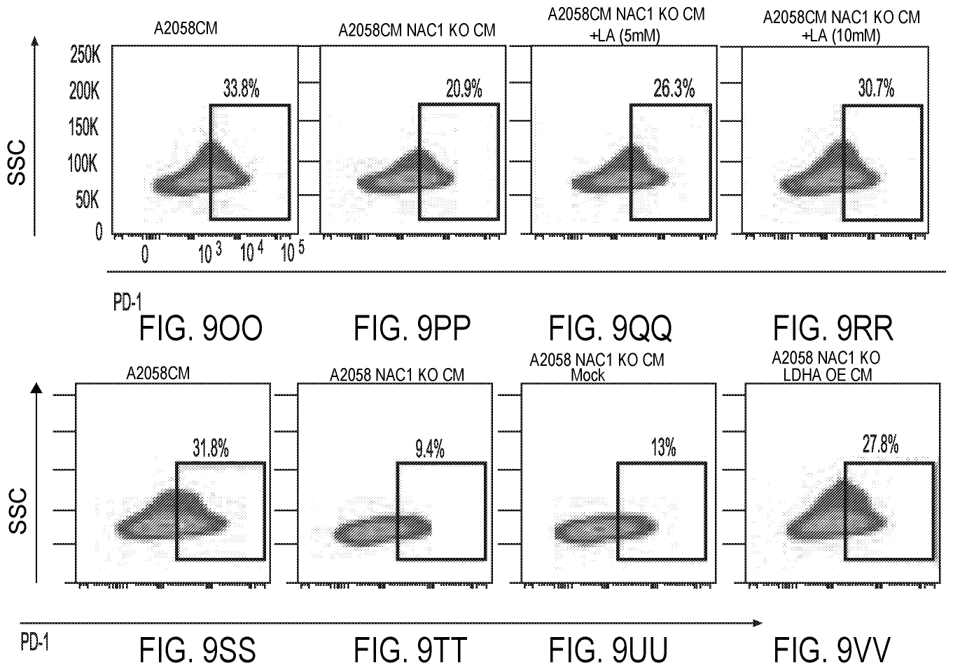

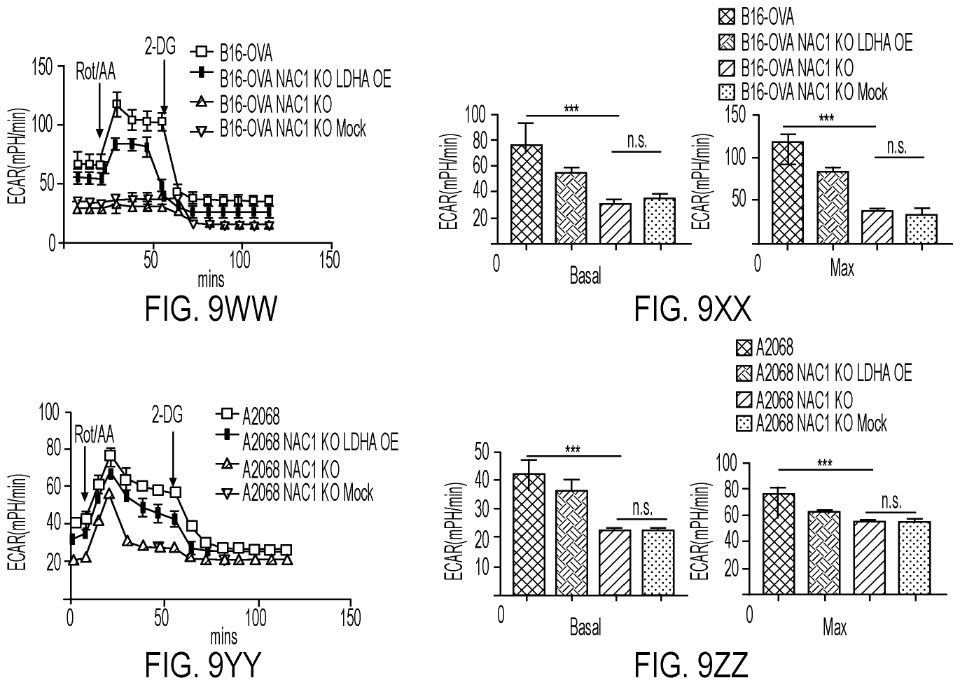

Next, mouse CD8⁺ T cells in the CM from NAC1 KO mouse B16-OVA cells were cultured and the medium was supplemented with or without 2 mM or 5 mM LA. The addition of LA reduced the production of cytokines including TNF-alpha and IFN-gamma and increased the expression of PD-1 and TIM-3 of CD8⁺ T cells (FIGS. 9A-9D). The addition of LA also caused a higher percentage of apoptosis determined by Annexin V staining (FIGS. 9E-9H). In parallel, NAC1 KO tumor cells were transfected with an LDHA expression retroviral vector or a retroviral backbone to overexpress LDHA in NAC1 KO B16-OVA. The mouse CD8⁺ T cells in the CM were cultured from NAC1 KO B16-OVA cells overexpressing LDHA (NAC1 KO LDHA OE) or an empty control (NAC1 KO Mock). The overexpression of LDHA conferred NAC1 KO mouse tumor cells with the ability to suppress the production of cytokines and increased expression of PD-1 (FIGS. 9I-9L) and apoptosis (FIGS. 9M-9P) of mouse cytotoxic CD8⁺ T cells. Similar results were obtained from human CD8⁺ T cells in the CM from NAC1 KO human A2058 cells, NAC1 KO human A2058 cells supplemented with the medium, with or without LA (5 mM or 10 mM), or NAC1 KO human A2058 cells overexpressing LDHA (NAC1 KO LDHA OE) (FIGS. 9Q-9VV).

To further prove that LDHA plays a role in metabolic reprogramming in melanoma cells expressing NAC1, their glycolysis profile of these mouse or human tumor cells (WT, NAC1 KO, NAC1 KO Mock, NAC1 KO LDHA OE) were analyzed and compared. Overexpression of LDHA (LDHA-OE) could partially restore glycolytic activity in the NAC1 KO mouse B16-OVA cells (FIGS. 9WW-9XX) and human A2058 tumor cells (FIGS. 9YY-9ZZ). Taken together, these results indicate that NAC1-mediated LDHA expression plays a critical role in supporting an immune-suppressive TME through its promotion of LA production.

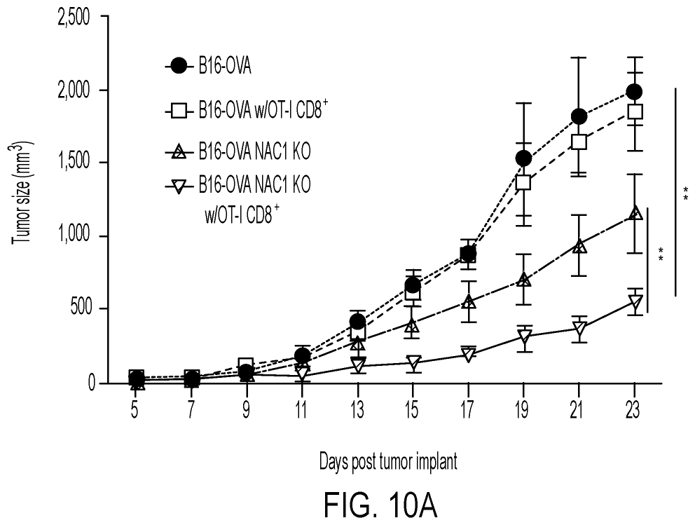

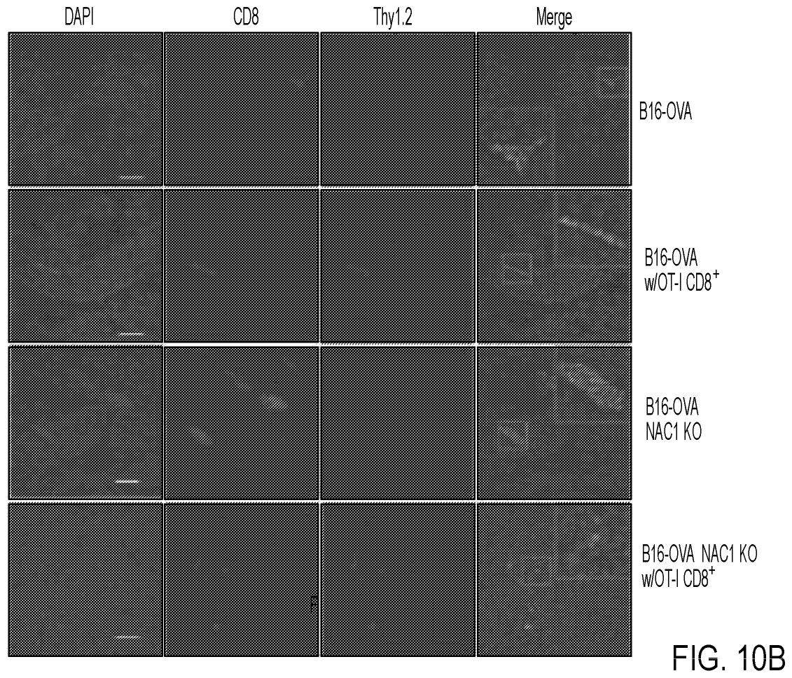

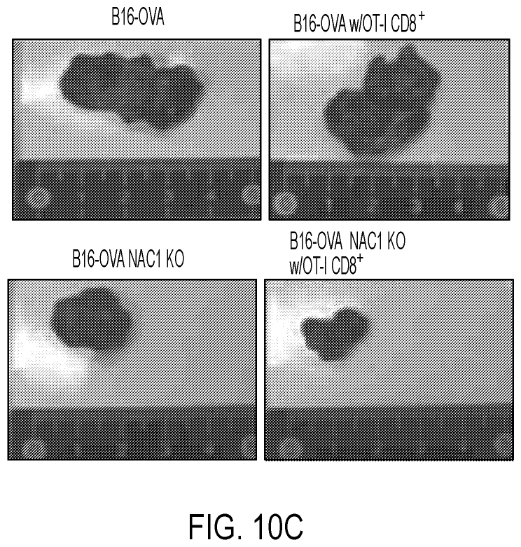

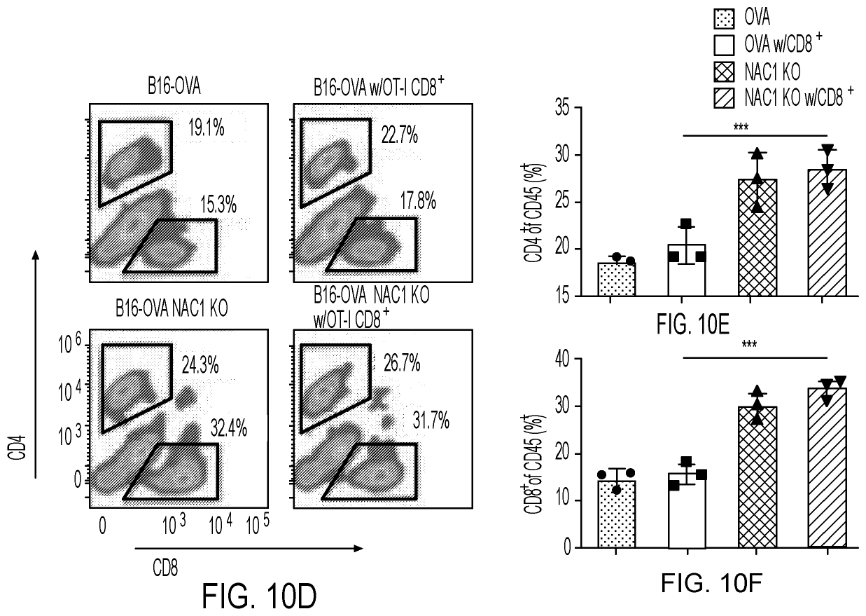

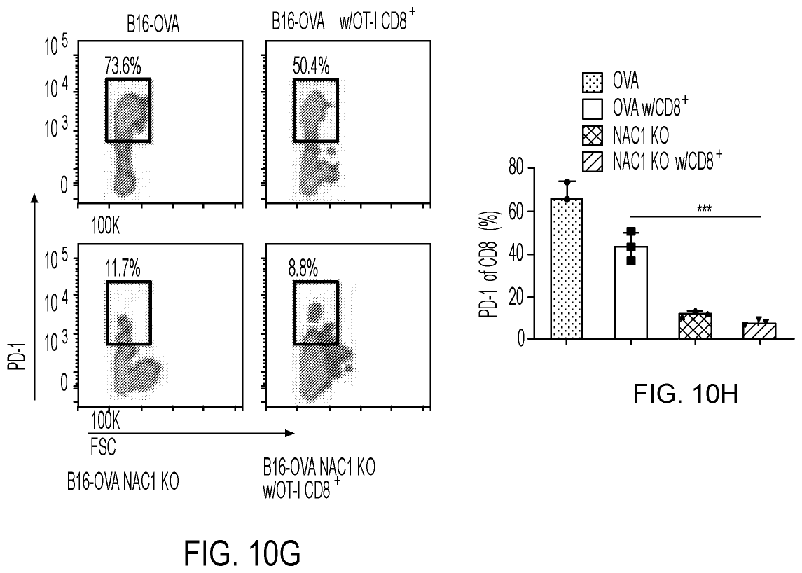

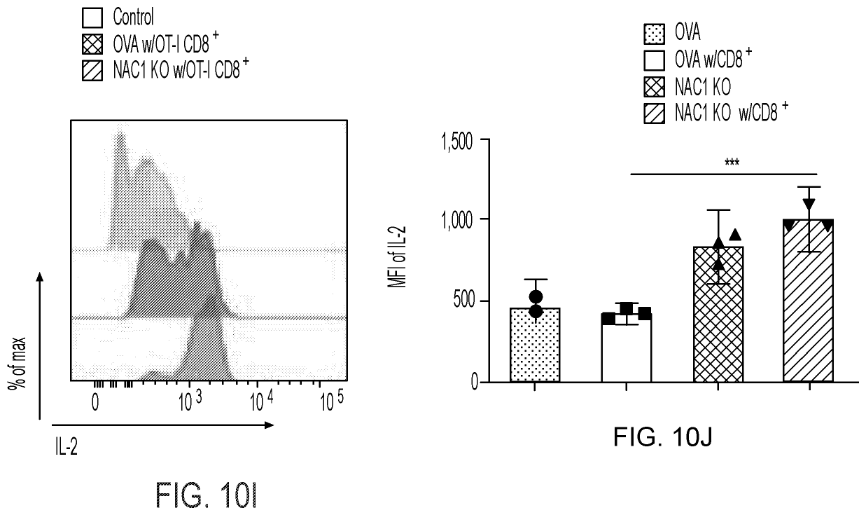

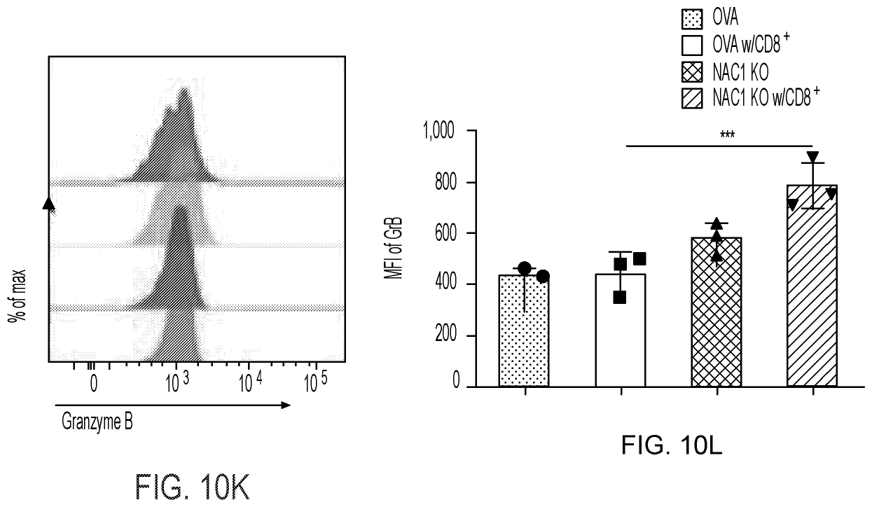

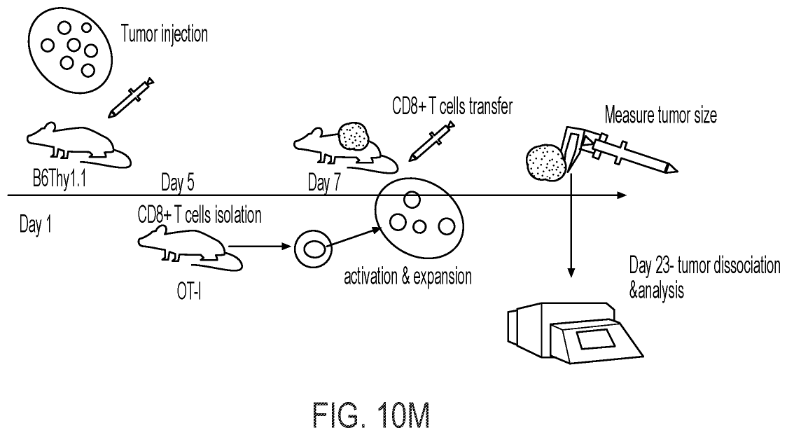

FIGS. 9A-9Z and 9AA-9ZZ demonstrate that effects of LDHA expression and lactic acid level on cytokine production, apoptosis, and exhaustion of CD8⁺ T cells. Mouse or human CD8⁺ T cells were incubated with CM from mouse WT or NAC1 KO B16-OVA cells, or human WT or NAC1 KO A2058 tumor cells for 24 hours. In parallel, some T cells were incubated with CM from NAC1 KO tumor cells overexpressing LDHA (NAC1 KO LDHA OE) or an empty control (NAC1 KO Mock), or CM supplemented with LA (2-5 mM for mouse T cells; 5-10 mM for human T cells). FIGS. 9A-9D are graphical representations of the production of cytokines TNF-α (FIG. 9A) or IFN-γ (FIG. 9B) and expression of PD-1 (FIG. 9C) and TIM-3 (FIG. 9D) of mouse CD8⁺ T cells with CM from mouse WT or NAC1 KO B16-OVA cells, or CM supplemented with LA by flow cytometry. One-way ANOVA with multiple comparisons correction. *p≤0.05, **p≤0.01. FIGS. 9E-9H are representations of the flow cytometry analysis of the apoptosis of mouse CD8⁺ T cells with CM from mouse WT (FIG. 9E) or NAC1 KO B16− OVA cells (FIG. 9F), or CM supplemented with LA (FIG. 9G and FIG. 9H). FIGS. 9I-9L are graphical representations of the production of cytokines IFN-γ (FIG. 9I) or TNF-α (FIG. 9J) and expression of Granzyme B (FIG. 9K) and PD-1 (FIG. 9L) of mouse CD8⁺ T cells with CM from mouse WT or NAC1 KO B16-OVA cells, or CM from NAC1 KO tumor cells overexpressing LDHA or an empty control by flow cytometry. One-way ANOVA with multiple comparisons correction. **p≤0.01. FIGS. 9M-9P are representations of the flow cytometry analysis of the apoptosis of mouse CD8⁺ T cells with CM from mouse WT (FIG. 9M) or NAC1 KO B16-OVA cells (FIG. 9N), or CM from NAC1 KO tumor cells overexpressing LDHA (FIG. 9O) or an empty control (FIG. 9P) by Annexin V staining. FIGS. 9Q-9X are representations of the analysis of the apoptosis of human CD8⁺ T cells with CM from human WT (FIG. 9Q and FIG. 9U) or NAC1 KO A2058 cells (FIG. 9R and FIG. 9V), or CM supplemented with 5 mM LA (FIG. 9S) or 10 mM LA (FIG. 9T), or CM from human NAC1 KO tumor cells overexpressing LDHA (FIG. 9W) or an empty control (FIG. 9X) by Annexin V staining. FIGS. 9Y-9FF are representations of the analysis of the production of IFN-γ of human CD8⁺ T cells with CM from human WT (FIG. 9Y and FIG. 9CC) or NAC1 KO A2058 cells (FIG. 9Z and FIG. 9DD), CM supplemented with 5 mM LA (FIG. 9AA) or 10 mM LA (FIG. 9BB), or CM from human NAC1 KO tumor cells overexpressing LDHA (FIG. 9EE) or an empty control (FIG. 9FF). FIGS. 9GG-9NN are representations of the analysis of the production of IL-2 of human CD8⁺ T cells with CM from human WT (FIG. 9GG and FIG. 9KK) or NAC1 KO A2058 cells (FIG. 9HH and FIG. 9LL), CM supplemented with 5 mM LA (FIG. 9II) or 10 mM LA (FIG. 9JJ), or CM from human NAC1 KO tumor cells overexpressing LDHA (FIG. 9MM) or an empty control (FIG. 9NN). FIGS. 9OO-9VV are representations of the analysis of the expression of PD-1 of human CD8⁺ T cells with CM from human WT (FIG. 9OO and FIG. 9SS) or NAC1 KO A2058 cells (FIG. 9PP and FIG. 9TT), CM supplemented with 5 mM LA (FIG. 9QQ) or 10 mM LA (FIG. 9RR), or CM from human NAC1 KO tumor cells overexpressing LDHA (FIG. 9UU) or an empty control (FIG. 9VV). FIG. 9WW is a graphical representation of the ECAR of mouse tumor cells measured by the Seahorse Metabolic Analyzer. Data are represented as mean±SEM; n=6 per condition from two independent experiments. FIG. 9XX is a graphical representation of basal glycolysis rates (left panel) and maximum glycolysis rate (right panel) of mouse tumor cells. One-way ANOVA with multiple comparisons correction. ***p≤0.001. FIG. 9YY is a graphical representation of ECAR of human tumor cells measured by the Seahorse Metabolic Analyzer. Data are represented as mean±SEM; n=6 per condition from two independent experiments. FIG. 9ZZ is a graphical representation of basal glycolysis rates (left panel) and maximum glycolysis rate (right panel) of human tumor cells. One-way ANOVA with multiple comparisons correction. ***p≤0.001. ANOVA, analysis of variance; CM, conditional medium; ECAR, extracellular acidification rate; LDHA, lactate dehydrogenase A; NAC1, nucleus accumbens-associated protein-1; OVA, ovalbumin.

### Depletion of Tumorous NAC1 Enhances the Efficacy of ACT of CTLs Against Melanoma.

To evaluate the impact of tumorous expression of NAC1 on melanoma immunotherapy, C57BL/6 congenic (B6. Thy1.1⁺) mice were inoculated s.c. with either WT B16-OVA or NAC1 KO B16-OVA tumor cells (1×10⁶ cells/mouse). Inoculation was followed by providing the mice with or without an ACT of OT-I CTLs (5×106 cells/mouse; Thy1.2⁺). The tumor sizes in the mice bearing WT B16-OVA tumors were significantly larger than the animals bearing NAC1 KO B16− OVA tumors (FIG. 10A). Transfer of OT-I CTLs had little effect on tumor growth in the mice inoculated with WT B16-OVA cells; however, the CTL treatment markedly inhibited tumor growth in the mice bearing NAC1 KO B16-OVA tumors (FIGS. 10A, 10C). Moreover, all (100%) NAC1 KO B16-OVA tumor bearing mice receiving OT-I CTLs survived to Day 23, compared with WT B16-OVA untreated control (40%) or WT B16-OVA group of mice treated with OT-I CTLs (60%). The potent inhibitory effect of CTLs on the growth of NAC1 KO tumors may be attributed to increased infiltration of Thy1.2⁺CD8⁺ cells into tumor tissues (FIGS. 10B, and 10D-10F), less exhaustion of CTLs (as evidenced by low expression of PD-1) (FIGS. 10G-10H), and increased production of proinflammatory cytokines IL-2 and Granzyme B by CTLs (FIGS. 10I-10L).

FIGS. 10A-10M demonstrate NAC1 deficiency increases CTL infiltration in tumors following an ACT in immune-competent mice. Mouse WT B16-OVA or NAC1 KO B16-OVA tumor cells (1×106) were injected s.c. in the flank of B6. Thy1.1⁺ mice (n=5), followed by treatment with or without the ACT of OT-I CD8⁺ T cells. FIG. 10A is a graphical representation of the progression of tumor size from mouse WT B16-OVA or NAC1 KO B16-OVA tumor cells, followed by treatment with or without the ACT of OT-I CD8⁺ T cells. FIG. 10B is a set of immunofluorescence DAPI, CD8, Thy1.2, and a merged staining of OT-I CD8⁺ T cell infiltration (Thy1.2⁺CD8⁺) in tumors. FIG. 10C is a set of photographic images of the indicated tumors as harvested on day 23. FIG. 10D is a flow cytometric analysis of tumor-infiltrating CD4⁺ T cells and CD8⁺ T cells from WT B16-OVA or B16-OVA NAC1 KO mice, followed by treatment with or without the ACT of OT-I CD8⁺ T cells. FIGS. 10E and 10F are graphical representations the percentage of tumor-infiltrating CD4⁺ T cells and CD8⁺ T cells from WT B16-OVA or B16-OVA NAC1 KO mice, followed by treatment with or without the ACT of OT-I CD8⁺ T cells. FIGS. 10G and 10H are histograms showing expression of PD-1⁺ (FIG. 10G) and quantitation of PD-1⁺ cells of total infiltrated Thy1.2⁺ CD8⁺ cells (FIG. 10H). FIGS. 101 and 10J are histograms showing production of IL-2 (FIG. 10I) and quantitation of MFI of IL-2 of total infiltrated CD8⁺ cells (FIG. 10J). FIGS. 10K and 10L are histograms showing production of Granzyme B (FIG. 10L) and quantitation of MFI of Granzyme B of total infiltrated CD8⁺ cells (right panel). FIG. 10M is a schematic representation of experimental paradigm. B6 Thy 1.1 mice were injected with mouse or human tumor cells on Day 1 followed by antigen specific CD8⁺ T cell transfer on day 7. The tumor size was monitored until day 23 post injection of the melanoma cells. All the results shown are representative of three identical experiments. *p≤0.05, ***p≤0.001. ACT, adoptive cell transfer; CTL, cytotoxic T lymphocytes; MFI, mean fluorescence intensity; NAC1, nucleus accumbens-associated protein-1; OVA, ovalbumin.

To further demonstrate the importance of the tumorous expression of NAC1 in antitumor immunity, the humanized (NOD-scid IL2rg^{null}, NSG) mouse melanoma model was tested. In these experiments, NSG immune-compromised mice were inoculated s.c. with either human WT A2058 or NAC1 KO A2058 melanoma cells into their flanks. When tumor sizes reached 100 mm³, the mice were injected with the human tyrosinase-specific CTLs via the tail vein. Tumor growth was significantly suppressed in NAC KO tumor-bearing mice receiving an ACT of CTLs than those without the CTL treatment (FIGS. 11A-11C). Analysis of tyrosinase-specific CTL infiltration in tumor tissues was conducted using immunofluorescence microscopy (FIG. 11D) and flow cytometry (FIGS. 11E-11I). These analyses revealed more significant amounts of CTLs in NAC1 KO tumors than in NAC1-expressing tumors. In addition, the CTLs in NAC1 KO tumors showed substantially higher levels of the cytokines, including IFN-γ, IL-2, and granzyme B (FIGS. 11J-11N). Consistently, the CTL exhaustion observed in NAC1-expressing tumor cells was significantly improved when tumor cells lost NAC1, as suggested by the reduced levels of TIM-3 and PD-1 (FIGS. 11J-7K). Together, these results imply that the tumorous expression of NAC1 may contribute to immune evasion through an acidic TME caused by upregulation of the LDHA-mediated production of LA. Targeting NAC1 in tumor cells may restrict LA production and improve the TME, thereby reinforcing immunotherapy such as ACT-based immunotherapy (FIG. 11O).

FIGS. 11A-11O demonstrate NAC1 deficiency increases tumor CTL infiltration following an ACT in immune-deficient mice. Human WT A2058 or NAC1 KO A2058 tumor cells (1×106) were s.c. injected into the flank of NSG mice, followed by treatment with or without the ACT of Tyrosinase-specific CD8⁺ T cells. FIG. 11A is a graphical representation of the progression of tumor size. FIG. 11B is a graphical representation of the tumor weight as evaluated by scales and represented graphically. Values are mean±SEM of three independent experiments (n=5). FIG. 11C is a set of photographs of the indicated tumors as harvested and photographed at day 25. FIG. 11D is a set of photographs of the CD8⁺ T cell infiltration (CD8⁺; green) in tumors (tyrosinase; red) as determined by immunofluorescence staining. Images are representative images taken from the tumors of three different mice. FIGS. 11E-11H are flow cytometric analysis of infiltrating CD8⁺ T cells in WT (FIGS. 11E and 11F) and NAC1 KO mice (FIGS. 11G and 11H) with or without the ACT of Tyrosinase-specific CD8⁺ T cells, respectively. FIG. 11I is a graphical representation of the tumor infiltrating CD8⁺ T cells in WT and NAC1 KO mice with or without the ACT of Tyrosinase-specific CD8⁺ T cells, respectively. Values are the mean±SEM of 3 independent experiments (n=5). FIG. 11J is a graphical representation of the flow cytometric analysis of infiltrating TIM-3⁺ CD8⁺ T cells. FIG. 11K is a graphical representation of the flow cytometric analysis of infiltrating PD-1⁺CD8⁺ T cells. Values are the mean±SEM of 3 independent experiments (n=5). FIG. 11L is a graphical representation of the flow cytometric analysis of IFN-7 production of infiltrating CD8⁺ T cells. FIG. 11M is a graphical representation of the flow cytometric analysis of the IL-2 production of infiltrating CD8⁺ T cells. FIG. 11N is a graphical representation of the flow cytometric analysis of Granzyme B production of infiltrating CD8⁺ T cells. Values are the mean±SEM of four independent experiments (n=4). FIG. 11O is a schematic representation of the mechanism. **p≤0.01, ***p≤0.001. ACT, adoptive cell transfer; CTL, cytotoxic T lymphocytes; LDHA, lactate dehydrogenase A; MFI, mean fluorescence intensity; NAC1, nucleus accumbens-associated protein-1.

The efficacy of antitumor immunity is often hindered by a multitude of factors that contribute to immune evasion. Expression of NAC1 negatively regulates the suppressive activity of CD4⁺ Tregs and the formation of CD8⁺ memory T cells. Tumorous NAC1 is a critical determinant of antitumor immunity, and NAC1 promotes immune evasion through LDHA-mediated production of LA, contributing to an acidic and immune-suppressive TME. Metabolic reprogramming of immune cells in the TME can impact antitumor therapeutic outcomes. A few connections between glycolytic metabolism and T cell regulation have been revealed. As the central players in the ACT, T cells develop rapid immune response through several stages, including initial cell growth followed by massive clonal expansion and differentiation, a contraction or death phase, and establishment and maintenance of immune memory. Metabolic reprogramming has important roles in these processes. During the initial growth phase, T cells undergo an activation-induced metabolic reprogramming, switching from the β-oxidation of fatty acids in T cells to glycolysis, pentose-phosphate and glutamino-lytic pathways in activated T cells. This phase represents the engagement of biosynthesis to produce proteins, nucleic acids, lipids, carbohydrates, and other macro-molecules for generation of new cells. Activated T cells upregulate glycolysis for their growth, proliferation, and function, but inhibiting glycolysis was reported to enhance CD8⁺ T cell memory and antitumor function. Whereas CD8⁺ T cells differentiate into CTLs, CD4⁺ T cells differentiate into either induced T_{regs} (iT_{regs}) that suppress uncontrolled immune responses or cells of the TH1, TH2 or TH17 subset of T cells (effector T cells, Teffs) that mediate appropriate immune responses. Glycolysis promotes CD4⁺ T cell differentiation into various Teffs, and inhibition of glycolytic activity blocks this process but promotes Tregs. Glycolysis is also critical for function of Teffs. Of these various T cell subsets, the iT_{regs} and memory T cells, mainly rely on lipid oxidation as a major source of energy, whereas CTLs and Teffs sustain high glycolytic activity and glutaminolytic activity. Lipid metabolism is believed to be the key metabolic pathway in Treg development and differentiation. Dendritic cells and macrophages also switch to glycolysis on activation even though they do not proliferate. Thus, metabolic reprogramming in various immune cells is intimately associated with their differentiation, survival, and function; yet the molecular mechanisms and pathways involved remain to be fully explored. Based on the functional association between metabolic reprogramming and T cell activation, expansion, and effector function, targeting T cell metabolism provides new directions to modulate therapeutic immunity.

Metabolic reprogramming in tumor cells also impacts antitumor immunity. One of the hallmarks of cancer is a metabolic reprogramming, which supports macromolecule synthesis, bioenergetics demand, and cellular survival. TME may be severely affected by the metabolic status of tumor cells, and can be such that even the most potent immune cells can accomplish their cytocidal function. The potency of ACT is impacted by metabolic alteration of tumor cells, which have the capability to cope by enhancing alter-native energy production mechanisms. While normal cells rely on respiration, malignant cells depend on glycolysis even in the presence of sufficient oxygen. Warburg metabolism consumes glucose and increases the production of LA, and this altered cellular metabolism causes the changes in the nutrient compositions in the TME. As both tumor and tumor stromal cells have high glycolytic activity, through secretion of lactate they can build an acidic TME that affects the development and function of immune cells. For instance, massive production of LA from tumor cells was reported to inhibit T cell cytotoxicity and effector functions. Tumor cells consume glucose more competently than T cells, and this deprives TILs of this key nutrient and thus weakens the cytotoxic functions of CD8⁺ T cells. Alterations in tumor cell metabolism can deprive TILs of essential nutrients that are required for effective response to the tumor cells, leading to immune evasion. The glucose-deficient TME may diminish CTLs activity through an immunosuppressive TME, and tumor cells with higher glycolytic activity have a strong capacity to evade immunosurveillance. On the other hand, cancer cells themselves can become impervious to cytocidal effects of ACT via reprogramming energy metabolism. Also, decreased expressions of tumor Ags and major histocompatibility complexes and increased expressions of inhibitory checkpoint molecules (e.g., PD-L1) may provide a growth advantage to tumor cells through evading T cell-mediated immune destruction. It was reported that Warburg glycolysis was associated with tumor cell resistance to TNF-related apoptosis-inducing ligand (TRAIL-induced cell death and chemotherapeutic agents such as paclitaxel and doxorubicin. Targeting the eukaryotic elongation factor-2 kinase-mediated glycolysis can sensitize cancer cells to paclitaxel and doxorubicin, and depletion of this kinase increases tumor cell sensitivity to TRAIL, curcumin, velcade, temozolomide, and AKT inhibitors via activating apoptosis. NAC1 plays an important role in regulating glycolysis and hypoxia response. Expression of NAC1 negatively regulates the suppressive activity of CD4⁺ Tregs and the formation of CD8⁺ memory T cells. Although several pathways, including NAC1, are known to promote metabolic reprogramming in tumor cells, whether and how they affect antitumor immunity remains largely unclear.

The antitumor immune response induced by CTLs can be weakened by acidification of the TME through the metabolic reprogramming of tumor cells. Practical approaches to restrain tumorous production of lactate may improve immune permissive-TME and strengthen immunotherapy. A recent study showed that bicarbonate administration to neutralize the acidic TME is a promising strategy to improve the efficacy of adoptively T cell transfer-based immune-therapy. Here, high expression of NAC1 in melanoma cells is associated with poor prognosis of melanoma patients and reduced cytotoxicity of CTLs (FIGS. 5 and 6). The effect of NAC1 on antitumor immunity is mediated through LDHA-regulated LA production (FIGS. 7 and 8).

LDHA-mediated LA production restrained CTL activity and function, and inhibitors of LDHA can strengthen the antitumor activity of CTLs both in vitro and in vivo. NAC1 regulates LDHA expression, as well as LA production in melanoma cells, may provide NAC1 a new target for modulating the TME, suppressing immune evasion, and enhancing the efficacy of ACT. Notably, both immune-competent B6. Thy1.1 mouse model and immune-deficient NSG mice bearing human melanoma show that depletion of tumor NAC1 significantly enhances the therapeutic efficacy of ACT (FIG. 10 and FIG. 11) by improving the TME and invigorating CTLs. Because the status of these immune cells can also be affected by the glycolytic activity of tumor cells, in addition to improving the TME and invigorating CTLs, targeting NAC1 may modulate other immune cell-mediated antitumor immunity.

Successful cancer immunotherapy could be hindered by the barriers such as low amount of tumor Ag-specific T cells due to clonal erasure, poor activation of T cells, accumulation of tolerogenic Ag-presenting cells in the TME, and formation of a hypoxic and immuno-suppressive TME. Notably, studies have indicated that the metabolic status of both immune cells and tumor cells can have a great impact on antitumor immunity. Immune cells and tumor cells can upregulate glycolysis, the anaerobic metabolism of glucose into ATP, when they turn into a highly proliferative state, to meet their needs for large amounts of energy as building materials. Immune activation, acquisition of effector functions, and generation of immune memory are closely coupled with alterations in energy metabolism. In particular, the transition from quiescence to activation is associated with a significant and prolonged escalation of aerobic glycolysis (Warburg effect), which can supply ATP, glycolytic intermediates for biosynthesis of DNA and cellular structural materials. NAC1 is a key modulator of tumor immune evasion and as demonstrated herein, its role is mediated through the LDHA-regulated production of LA. TME consists of tumor cells, tumor stromal cells and various immune cells. In addition to impact CTLs, NAC1 may play roles in other immune cells. Nevertheless, targeting NAC1 in tumor cells represents a novel strategy that significantly strengthens the adoptive T cell transfer-based cancer immunotherapy.

Embodiments include a method of reducing NAC1 expression or activity in a cancer patient includes administering to the cancer patient a chemical agent or a biological agent to inhibit the function of NAC1, along with an adoptive cell transfer therapy. Certain embodiments include providing a composition containing a nucleotide or a peptide-based agent to inhibit the function of NAC1, along with an adoptive cell transfer therapy. Certain embodiments include providing a composition containing NAC1-targeted siRNA nanoliposomes, along with an adoptive cell transfer therapy. Certain embodiments include providing a CRISP/Cas9 composition that suppresses or knockdowns NAC1, along with an adoptive cell transfer therapy. Certain embodiments include providing a composition containing an isolated antibody or its binding fragment thereof that binds to NAC1, along with an adoptive cell transfer therapy. Adoptive cell transfer therapy includes a chimeric antigen receptor T-cell (CAR T-cell) therapy or a tumor-infiltrating lymphocyte (TIL) therapy. In each of the foregoing embodiments, the inhibitor of NAC1 can be administered before, after, or concurrent with the adoptive cell transfer therapy.

Embodiments include a method of reducing NAC1 expression or activity in a cancer patient includes administering to the cancer patient (a) one or more DNA sequences encoding one or more guide RNAs (gRNAs) that are complementary to one or more target sequences in a NAC1 gene and (b) a nucleic acid sequence encoding a Clustered Regularly Interspaced Short Palindromic Repeat (CRISPR)-associated endonuclease, whereby the one or more gRNAs hybridize to the NAC1 gene and the CRISPR-associated endonuclease cleaves the NAC1 gene. The NAC1 sequence can be deleted in the patient relative to a patient to whom the CRISPR/Cas-based genome editing composition is not administered. The NAC1 expression or activity can be reduced in the patient relative to a patient to whom the CRISPR/Cas-based genome editing composition is not administered. In some embodiments, the one or more gRNAs are complementary to a target sequence in the NAC1 gene. In some embodiments, the one or more gRNAs comprise a trans-activated small RNA (tracrRNA) and a CRISPR RNA (crRNA) In some embodiments, the one or more gRNAs are one or more single guide RNAs. In some embodiments, the CRISPR-associated endonuclease is a class 2 CRISPR-associated endonuclease, and in some embodiments, the class 2 CRISPR-associated endonuclease is Cas9 or Cas12a. In some embodiments, expression of one or more allele(s) of the NAC1 gene is reduced in the cancer patient. In some embodiments, NAC1 activity is reduced in the cancer patient. In some embodiments, NAC1 expression or activity is not substantially eliminated in the cancer patient. In some embodiments, NAC1 expression or activity is substantially eliminated in the cancer patient. Embodiments include a method of reducing NAC1 expression or activity in a cancer patient that further includes introducing a chimeric antigen receptor T-cell (CAR T-cell) therapy or a tumor-infiltrating lymphocyte (TIL) therapy.

NAC1^{-/-} Tregs show enhanced suppressive function. The co-transfer of Tregs with tumor cells could enhance tumor growth by the suppression of the host antitumor immunity by Tregs. Because NAC1^{-/-} Tregs were more potent in the suppression than WT Tregs, tumor growth was faster and animal survival was shorted in hosts receiving NAC1^{-/-} Tregs with tumor cells than in hosts receiving WT Tregs with tumor cells.

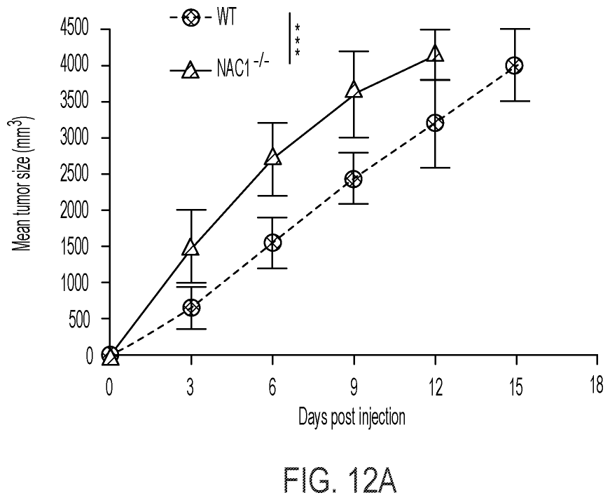

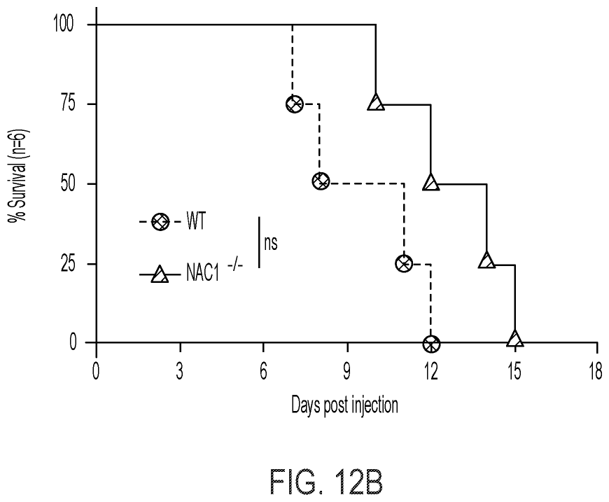

FIGS. 12A-12B demonstrate that NAC1^{-/-} Tregs show enhanced suppressive function. WT or NAC1^{-/-} Tregs (1×105) were injected s.c. in the flank region of the recipient mice with 1×106 B16 tumor cells on various days. FIG. 12A is a graphical representation of the tumor growth following injection of WT or NAC1^{-/-} Tregs. Data shown are the mean S.E.M. of tumor sizes of a representative of three identical experiments (N=6). ***, P<0.0001, simple linear regression. FIG. 12B is a graphical representation of the survival curves following injection of WT or NAC1^{-/-} Tregs. Data shown are the representative of three identical experiments (N=6). ns, P>0.05, Log-rank (Mantel-Cox) test.

### NAC1 and Vaccines

NAC1 modulates the functional activity of regulatory T cells (Tregs). NAC1 also plays a key role in the regulation of T cell memory formation. NAC1 has important roles in regulating CD8⁺ T cell function, survival, and memory. Interferon Regulatory Factor (IRF4), a transcription factor that is closely associated with T cell receptor (TCR) signaling, is involved in this regulation.

### Generation of Vaccinia Virus (VACV) as the Tool for Experiments on T Cell Memory Formation.

The VACV construct was developed as the tool for experiments on T cell memory formation because VACV can create a relatively strong and long-lasting T cell memory. HeLa cells were infected with VACV according to the VACV stock preparation protocol. HeLa cells altered their morphology 2 days post-infection. HeLa cells shrank in size and became more spherical, resulting in a less attached status. After the collection of VACV stock, a plaque assay was used to quantify the VACV titer. Following serial dilutions, the 10⁻⁷ dilution was found to provide a reliable plaque number. Quantification of the plaque assays showed that the virus titer was 3.85×10⁸ PFU/mL.

Defects in the Survival of NAC1^{-/-} CD8⁺ T Cells.

NAC1 has been shown to regulate cancer cell survival. Here, it was investigated whether NAC1 could interfere with T cell proliferation and survival. Cell proliferation between CD8⁺ T cells from WT and NAC1^{-/-} mice were compared. Naïve CD8⁺ T cells from the pooled LNs and spleen were labeled with carboxyfluorescein succinimidyl ester (CFSE) and stimulated with plate-coated anti-CD3 plus soluble anti-CD28 Abs, and then cell proliferation was determined by CFSE dilution. NAC1^{-/-} CD8⁺ T cells almost retained similar proliferation compared with the WT and NAC1^{-/-} mice. Three days after activation, NAC1^{-/-} CD8⁺ T cells doubled their population, but WT CD8⁺ T cells showed an almost 3-fold increase. On day 4, NAC1^{-/-} CD8⁺ T cells decreased their population, whereas WT T cells had an 8-fold increase in cell number compared with that on day 0. Moreover, 4 days later, WT CD8⁺ T cells also maintained robust survival as compared with NAC1^{-/-} CD8⁺ T cells. These results indicate that loss of NAC1 negatively affects the survival of CD8⁺ T cells.

Defects in Glycolysis and Oxidative Phosphorylation Rate of NAC1^{-/-} CD8⁺ T Cells.

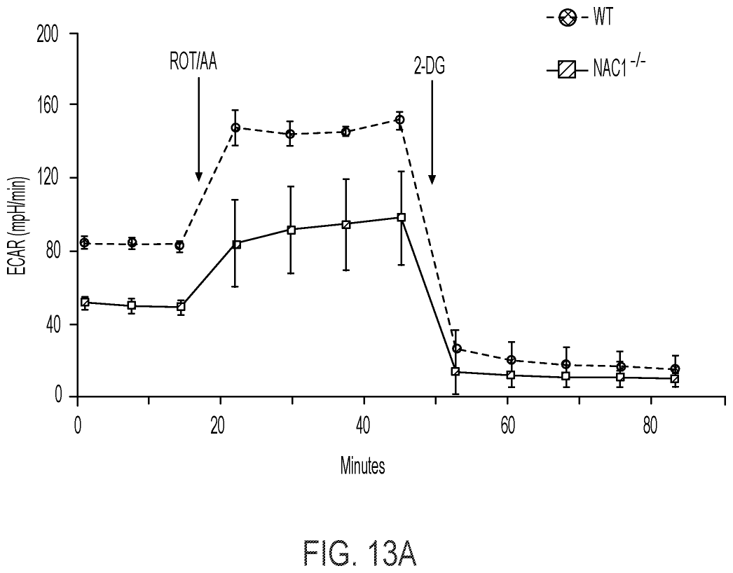

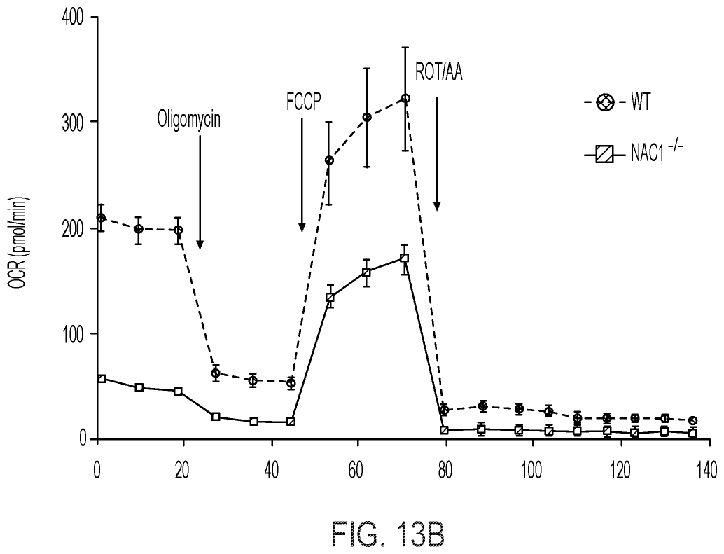

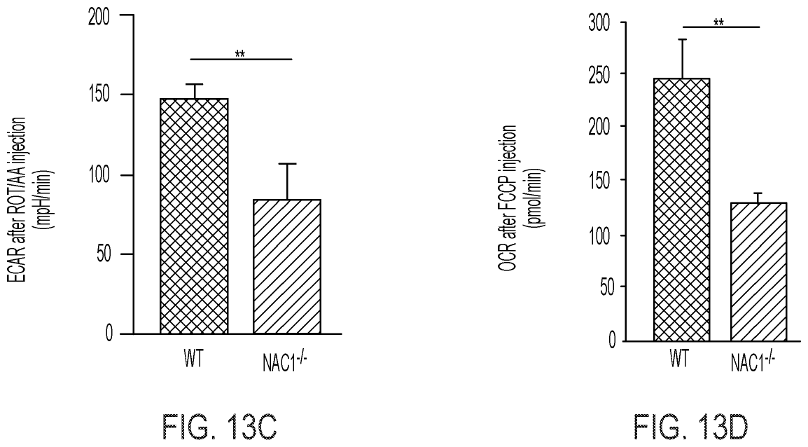

As NAC1 can regulate tumor cellular metabolism, this transcription co-regulator was hypothesized to play a role in T cell metabolism. To test this hypothesis, an Agilent Seahorse Assay was used to analyze glycolysis and oxidative phosphorylation in T cells. Following the addition of rotenone and antimycin A (Rot/AA), NAC1^{-/-} CD8⁺ T cells were observed to have a lower ECAR than WT cells (FIG. 13A, 13C), indicating that NAC1^{-/-} CD8⁺ T cells have a reduced glycolytic rate. After adding carbonyl cyanide-p-trifluoromethoxyphenylhydrazone (FCCP), a potent uncoupler of mitochondrial oxidative phosphorylation which disrupts ATP synthesis by transporting protons across cell membranes, NAC1^{-/-} CD8⁺ T cells had lower OCR than WT cells (FIG. 13B, 13D). These results indicate that after activation, NAC1^{-/-} CD8⁺ T cells have a reduced metabolic rate likely due to metabolic reprogramming caused by loss of NAC1.

FIGS. 13A-13D illustrate the defects in glycolysis and oxidative phosphorylation rate of NAC1^{-/-} CD8⁺ T cells. Cell metabolism was tested using the Agilent Seahorse Assay on day 3 after activation. (A, C) The cells were activated with coated anti-CD3 and soluble anti-CD28 Abs. Then, 2×10⁵ cells were plated in each well of seahorse microplate for glycolytic rate testing. The drugs were injected into each well at the time indicated in the figures. FIG. 13A is a graphical representation of the Extra Cellular Acidification Rate (ECAR), which was measured as a proxy for glycolytic rate. FIG. 13C is a graphical representation of the ECAR level after ROT/AA injection as compared between WT and NAC1^{-/-} groups. Sample numbers (n=4), the p-value is 0.0076. The activated 2×10⁵ T cells were seeded in a microplate before mitochondrial stress testing. The different drugs were added to the well according to the indicated time points as shown in FIG. 13B. FIG. 13B is a graphical representation of the Oxygen Consumption Rate (OCR), which was tested as a proxy for oxidative phosphorylation. The OCR level was then compared after FCCP injection between the two groups. FIG. 13D is a graphical representation of the OCR level after FCCP injection as compared between WT and NAC1^{-/-} groups. Sample numbers (n=4), p-value is 0.0054 (**, p≤0.01).

Sustained Survival of the VACV-Specific CD8⁺ T Cell in NAC1^{-/-} Mice.

How NAC1 influences antigen (Ag)-specific cell generation and survival in vivo was assessed. To monitor the Ag-specific T cell proliferation and survival in vivo, the mice with VACV (2×10⁶ PFU/mouse) were challenged. The number and frequency of the VACV-specific CD8⁺ T cells were determined in the next 5 weeks. Both WT and NAC1^{-/-} mice responded to VACV infection.

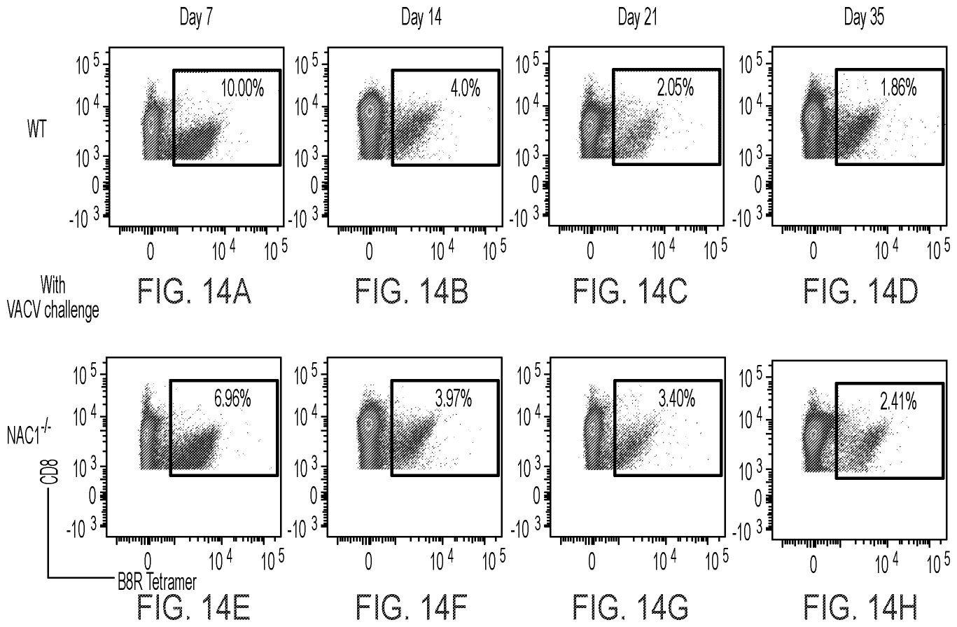

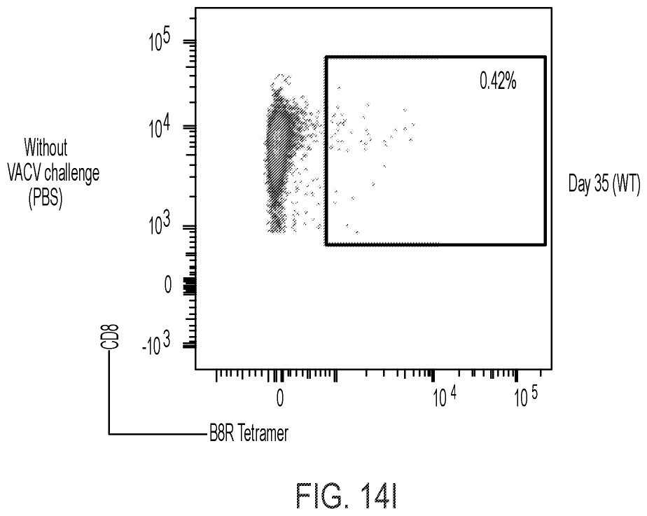

FIGS. 14A-141 provide the comparison of development of viral Ag-specific CD8⁺ T cells. The VACV-specific CD8⁺ T cell frequency was monitored in WT or NAC1^{-/-} mice for 35 days after VACV challenge. Mice were challenged with VACV at 2×10⁶ PFU/mouse. The spleen and LNs (superficial cervical, axillary, brachial, and inguinal nodes) were dissected, smashed, and stained with CD8 Ab and B8R tetramer. On day 7, day 14, day 21, and day 35, Ag-specific cell frequencies were analyzed by flow cytometry. FIGS. 14A-14H are flow cytometry analyses of CD8 Ab and B8R tetramer in WT (FIGS. 14A-14D) or NAC1^{-/-} mice (FIGS. 14E-14H) on day 7, day 14, day 21, and day 35 after VACV challenge. VACV-uninfected mice were used as a control. The spleen and lymph nodes were also dissected, smashed, and stained with CD8 Ab and B8R tetramer. FIG. 141 is a flow cytometry analysis of CD8 Ab and B8R tetramer in VACV-uninfected mice. The plotting data shown are representative of three identical experiments (n=5).

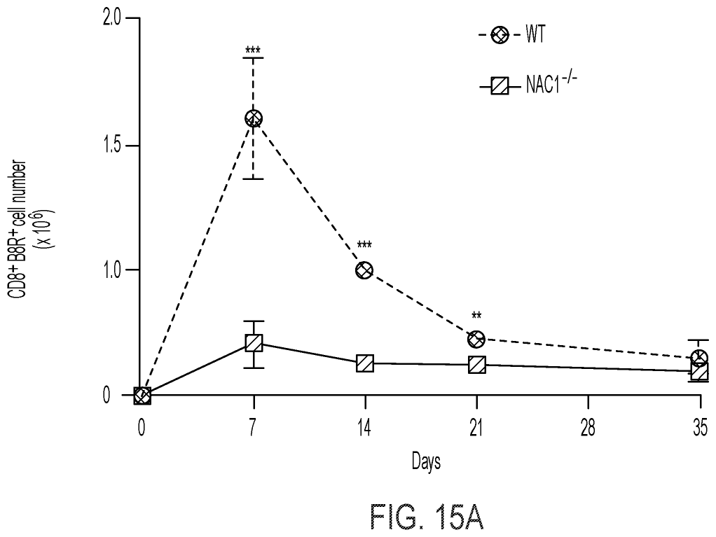

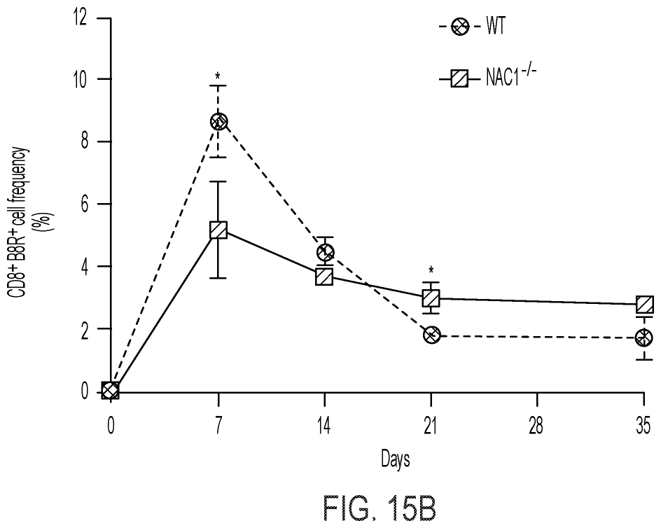

The T cell responses peaked on day 7 after the VACV challenge, then started to decline. Levels of IFNγ secretion were higher in WT CD8⁺ B8R⁺ T cells, but no obvious difference was observed for TNFα cytokine secretion. The VACV-specific CD8⁺ T cell number was significantly higher in WT than in NAC1^{-/-} mice during the initial 3 weeks (FIG. 15A). The VACV-specific CD8⁺ T cell numbers in both groups were decreased to a similar level on day 35 after viral infection (FIG. 15A). Although NAC1^{-/-} mice revealed a lower number of VACV-specific CD8⁺ T cells, its cell frequency in these mice was not continually lower than that in the WT group (FIG. 15B). In fact, after one week, the frequency of the VACV-specific CD8⁺ T cells decreased more slowly in the NAC1^{-/-} mice than that in the WT controls. In contrast, on day 21, NAC1^{-/-} mice maintained a higher frequency of the VACV-specific CD8⁺ T cells than WT controls. There was no significant difference in the VACV-specific CD8⁺ T cell frequency between those two groups 35 days later. These results indicate that NAC1 negatively affects the survival of the Ag-specific CD8⁺ T cell in vivo.

FIGS. 15A-15B demonstrate the sustained VACV-specific CD8⁺ T cell survival in NAC1^{-/-} mice. Mice were challenged with VACV at 2×10⁶ PFU/mouse. The spleen and lymph nodes (superficial cervical, axillary, brachial, and inguinal nodes) were dissected and smashed. The total live cell number for each mouse was calculated with trypan blue staining using a Bio-Rad cell counter. T cells were stained with CD8 Ab and B8R tetramer and analyzed by flow cytometry. Total Ag-specific CD8⁺ T cells were calculated for each mouse (n=5). FIG. 15A is a graphical representation of the VACV-specific CD8⁺ T cell number during the 35 days post-infection. The p-values are 0.00038 (day 7), 8.2×10⁻⁵ (day 14), and 0.0066 (day 21). The Ag-specific CD8⁺ T cell frequency was monitored for 35 days. FIG. 15B is a graphical representation of the VACV-specific CD8⁺ T cell frequency during the 35 days post-infection. P-values are 0.011 (day 7), and 0.016 (day 21). (*, p≤0.05; **, p≤0.01; ***, p≤0.001).

Enhanced CD8⁺ T Cell Memory Formation in NAC1^{-/-} Mice.

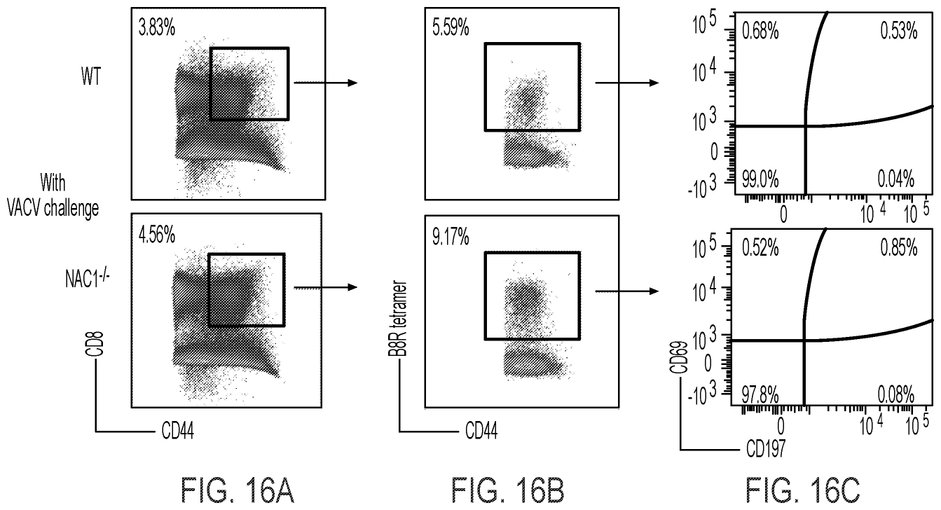

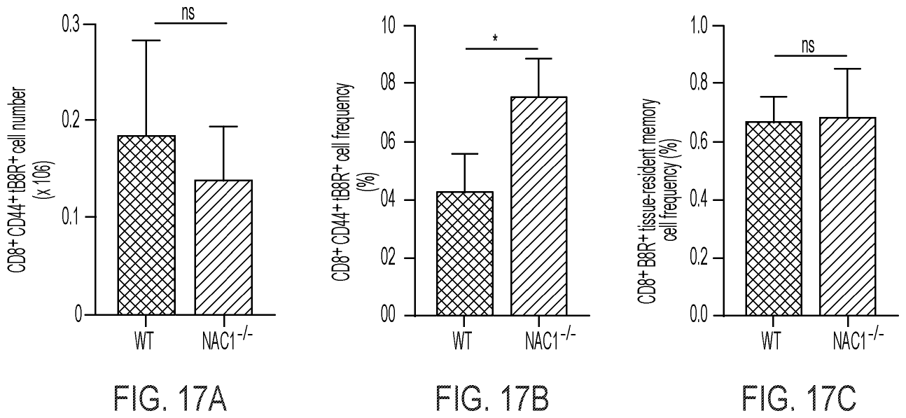

When a host eliminates a viral infection, memory T cells maintain homeostatic proliferation and survive past the initial immune response. To determine the effect of NAC1 on memory of CD8⁺ T cells, VACV was used to challenge mice and 35 days later, and the memory formation of T cells in both WT and NAC1^{-/-} mice were examined. A higher frequency of the VACV-specific memory CD8 T cells was observed in NAC1^{-/-} mice than that in WT controls, as analyzed by flow cytometry (FIG. 16B and FIG. 17B). Notably, there was no significant difference in the number of the VACV-specific memory CD8⁺ T cells between WT and NAC1^{-/-} groups (FIG. 17A), which was consistent with the Ag-specific CD8⁺ T cell number shown in FIG. 15A. Although the NAC1^{-/-} mice were less responsive to the VACV challenge and generated a smaller number of the VACV-specific CD8⁺ T cells in the early stage, their slowly decreased VACV-specific T cells resulted from the longer and better memory T cell formation after the effector stage. The memory T cell subsets (FIG. 16C) were further analyzed and found that a small portion of the tissue-resident memory (CD44^{hi}CD69^{hi}CD197^{low}) and central memory (CD44^{hi}CD69^{low}CD197^{hi}) CD8⁺ T cells existed, and most of them were effector memory (CD44^{hi}CD69^{low}CD197^{low}) T cells. There was no significant difference in the number of the tissue-resident memory T cells between NAC1^{-/-} and WT mice (FIG. 17C). On day 7 after VACV challenge, CD8⁺ B8R⁺ CD127⁺ CD62L⁺ (precursor) cells were higher in NAC1^{-/-} mice. For the terminally differentiated CD8⁺ B8R⁺ CD127⁻ KLRG1⁺ cells, that there was no significant difference relative to the wild-type. Based on these data, the higher memory precursor cells in NAC1^{-/-} mice are related to later higher memory T cell frequency. These results demonstrate that NAC1 suppresses memory CD8⁺ T cell formation in vivo.

FIGS. 16A-16C provide analyses of CD8⁺ T cell memory formation. T cell memory population was investigated in WT or NAC1^{-/-} mice after VACV challenge. The mice were sacrificed on day 35. The spleen and LNs were dissected, smashed, and stained before flow cytometry. FIG. 16A is a flow cytometry analysis of memory CD8⁺ T cells (CD8⁺ CD44⁺) in WT (top) or NAC1^{-/-} mice (bottom) after VACV challenge. FIG. 16B is a flow cytometry analysis of VACV-specific memory CD8⁺ T cells (CD8⁺CD44⁺ B8R⁺), gating on memory CD8⁺ T cells, in WT (top) or NAC1^{-/-} mice (bottom) after VACV challenge. FIG. 16C is a flow cytometry analysis of tissue-resident memory (CD44^{hi}CD69^{hi}CD197^{low}), central memory (CD44^{hi}CD69^{low} CD197^{hi}) and effector memory (CD44^{hi}CD69^{low}CD197^{low}) T cells, gating on VACV-specific memory CD8⁺ T cells, in WT (top) or NAC1^{-/-} mice (bottom) after VACV challenge. The plotting data shown are representative of three identical experiments (n=5).

FIGS. 17A-17C illustrate the enhanced CD8⁺ T cell memory formation in NAC1^{-/-} mice. Quantification for T cell memory population after 35 days. The mice were sacrificed on day 35. The spleen and lymph nodes were dissected and smashed. The total live cell number for each mouse was calculated with trypan blue staining through a Bio-Rad cell counter. T cell surface markers were stained before flow cytometry. FIG. 17A is a graphical representation of the VACV-specific memory CD8⁺ T cell (CD8⁺ CD44⁺ B8R⁺) number for each mouse, as calculated and compared between WT and NAC1^{-/-} groups. FIG. 17B is a graphical representation of the VACV-specific memory CD8⁺ T cell (CD8⁺ CD44⁺ B8R⁺) frequency for each mouse, as calculated and compared between WT and NAC1^{-/-} groups. The p-value is 0.040. FIG. 17C is a graphical representation of the tissue-resident memory (CD44^{hi}CD69^{hi}CD197^{low}) T cell frequency when analyzed between WT and NAC1^{-/-} groups. (*, p≤0.05; ns, p>0.05).

### Involvement of IRF4 in the NAC1-Mediated Restrain of CD8⁺ T Memory Formation.

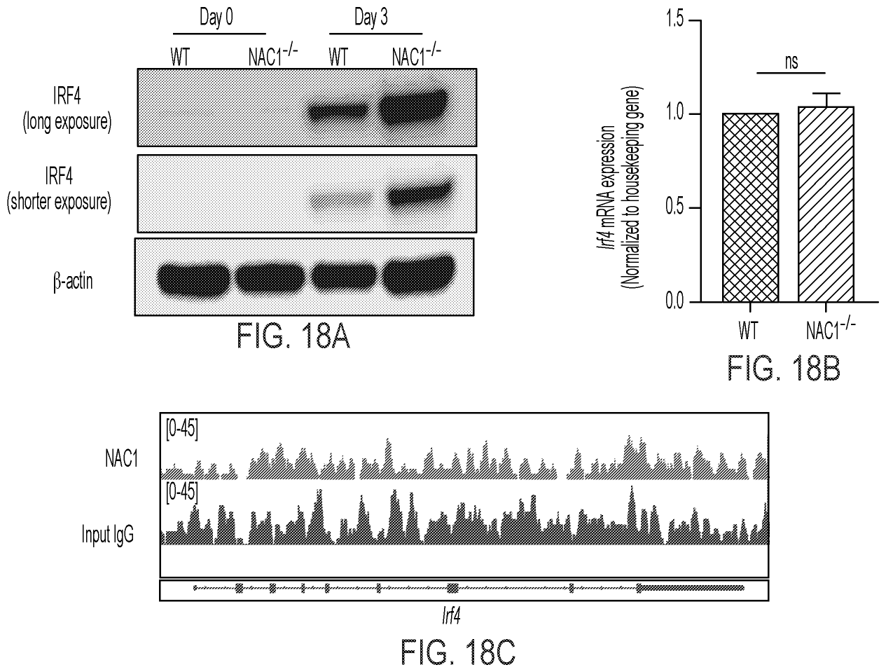

It was reported that IRF4 can support the resident memory CD8⁺ T cell maintenance and play a pivotal role in T cell activation. Therefore, the IRF4 expression in CD8⁺ T cells were examined. Naive CD8⁺ T cells were isolated from WT or NAC1^{-/-} mice and stimulated with plate-coated anti-CD3 plus soluble anti-CD28 Abs, and IRF4 protein expression was determined by Western blot. Before activation, the basal expression of IRF4 was barely detectable in both groups, but evidently increased after activation. Notably, on day 3, NAC1^{-/-} T cells had a much higher expression of IRF4 than the WT control (FIG. 18A), suggesting that NAC1 may control the expression of IRF4. However, there was no significant difference in the mRNA level for Irf4 between the two groups (FIG. 18B). In addition, the CHIP-seq analysis did not reveal any binding of NAC1 protein to Irf4 (FIG. 18C). Thus, the regulation of IRF4 by NAC1 does not occur at transcriptional level but at a post-transcriptional level.

FIGS. 18A-18C illustrate the regulation of IRF4 in CD8⁺ T cells by NAC1. CD8⁺ T cells were isolated from WT or NAC1^{-/-} mice and analyzed for expression of IRF4. FIG. 18A is a photographic representation of the Western blot analysis of IRF4 expression. For the day 0 sample, T cells were not activated. For the day 3 sample, T cells were activated and cultured for 3 days. FIG. 18B is a photographic representation of the Q-PCR analysis of mRNA expression. CD8⁺ T cells had been cultured for 3 days, and then RNA was extracted, and qPCR was performed (ns, p>0.05). FIG. 18C is a representation of the CHIP-seq analysis. WT CD8⁺ T cells were isolated from mice and CHIP was performed with anti-NAC1 and anti-IgG Abs (Input control). The sequenced data was visualized by IGV.

An animal model was used to investigate the regulation of CD8⁺ T cells by the NAC1 during viral infection. NAC1 controlled CD8⁺ T cell survival (FIGS. 16A-16C) and regulated cell metabolism (FIGS. 13A-13D). Using VACV, it was demonstrated that during the virus infection, loss of NAC1 sustained the survival of Ag-specific CD8⁺ T cells (FIGS. 15A-15B). At the recovery stage, NAC1^{-/-} CD8⁺ T cells maintained higher memory T cell formation (FIGS. 17A-17C) and IFR4 expression (FIGS. 18A-18C) than WT cells. These results indicate that NAC1 can repress CD8⁺ T cell memory formation and IRF4 is involved in this regulation.

NAC1 is important for tumor growth and metabolic reprogramming; consequently, targeting NAC1 can suppress tumor growth. For example, the NAC1 inhibitor, NIC3, can interrupt NAC1 homodimerization and shows antitumor activity. The role of NAC1 in T cell biology may be multifaceted, potentially playing different roles in different T cell subsets such as CD8, Th1, Th2, Th17, and Tregs. Loss of NAC1 impaired CD8⁺ T cell survival after activation in vitro and NAC1^{-/-} CD8⁺ T cells demonstrated decreased glycolysis after activation. Because T cells require different metabolic profiles during different stages of differentiation, NAC1-mediated alternations in glycolysis and oxidative phosphorylation may result in different T cell memory statuses during pathogen infections.

There are three important phases during T cell anti-viral immune response: activation and proliferation, death, and memory formation. NAC1 may differentially influence T cells during distinct phases after viral infection. NAC1 supported T cell survival. After viral infection, NAC1^{-/-} animals developed a smaller number of virus-specific CD8⁺ T cells. However, these virus-specific CD8⁺ T cells died slower than WT controls after the effector peak. After 35 days, NAC1^{-/-} animals maintained a higher frequency of virus-specific memory CD8⁺ T cells. These results indicate that NAC1 represses CD8⁺ T cell memory formation and that loss of NAC1 reduces the death of memory CD8⁺ T cells. Therefore, targeting NAC1 can be an effective approach to improving the effectiveness of some vaccines whose protection period is short. For example, the effectiveness of the COVID-19 vaccine had been proved to gradually decrease after 5-6 months among fully vaccinated people. Thus, it is advised that a booster should be given after 6 months. Alternatively, the decrease in protection can be slowed down by prolonging the lifetime and population of memory T cells to maintain vaccine effectiveness.

Embodiments include providing a chemical agent or a biological agent to inhibit the function of NAC1, along with administering a vaccine against an infection. Certain embodiments include providing a composition containing a nucleotide or a peptide-based agent to inhibit the function of NAC1, along with administering a vaccine against an infection. Certain embodiments include providing a composition containing NAC1-targeted siRNA nanoliposomes. Certain embodiments include providing a CRISP/Cas9 composition that suppresses or knockdowns NAC1, along with administering a vaccine against an infection. Certain embodiments include providing a composition containing an isolated antibody or its binding fragment thereof that binds to NAC1, along with administering a vaccine against an infection. In each of the foregoing embodiments, the inhibitor of NAC1 can be administered before, after, or concurrent with the vaccine. In some embodiments, expression of one or more allele(s) of the NAC1 gene is reduced in the subject. In some embodiments, NAC1 activity is reduced in the subject. In some embodiments, NAC1 expression or activity is not completely eliminated in the subject. In some embodiments, NAC1 expression or activity is completely eliminated in the subject.

Embodiments include a method of reducing NAC1 expression or activity in a subject who has received or is a receiving a vaccine against an infection by introducing into the subject (a) one or more DNA sequences encoding one or more gRNAs that are complementary to one or more target sequences in a variant NAC1 gene and (b) a nucleic acid sequence encoding a CRISPR-associated endonuclease, whereby the one or more gRNAs hybridize to the NAC1 gene and the CRISPR-associated endonuclease cleaves the NAC1 gene. The NAC1 sequence can be deleted in the patient relative to a patient to whom the CRISPR/Cas-based genome editing composition is not administered. The NAC1 expression or activity can be reduced in the patient relative to a patient to whom the CRISPR/Cas-based genome editing composition is not administered. In some embodiments, the one or more gRNAs are complementary to a target sequence in the NAC1 gene. In some embodiments, the one or more gRNAs comprise a trans-activated small RNA (tracrRNA) and a CRISPR RNA (crRNA). In some embodiments, the one or more gRNAs are one or more single guide RNAs. In some embodiments, the CRISPR-associated endonuclease is a class 2 CRISPR-associated endonuclease, and in some embodiments, the class 2 CRISPR-associated endonuclease is Cas9 or Cas12a. In some embodiments, expression of one or more allele(s) of the NAC1 gene is reduced in the subject. In some embodiments, NAC1 activity is reduced in the subject. In some embodiments, NAC1 expression or activity is not completely eliminated in the subject. In some embodiments, NAC1 expression or activity is completely eliminated in the subject.

In the setting of chronic infection, exhausted T cells express higher IRF4 and repress memory T cell formation. It has been reported that IRF4^{-/-} mice develop fewer memory CD8⁺ T cells due to their initially poor activation. Furthermore, in the conditional tamoxifen-induced Cre-lox knockout system, it was found that IRF4 supported resident memory T cell formation. Therefore, the roles of IRF4 in T cell memory formation are still controversial. Results herein show that NAC1 restrained overall CD8⁺ T cell memory formation but not the tissue-resident memory T cell population, and this was accompanied by down-regulation of IRF4 protein. Down-regulation of IRF4 appears to occur post-transcription, as the PCR analysis did not show significant difference in irf4 mRNA between WT and NAC1^{-/-} CD8⁺ T cells (FIG. 18). Taken together, this study demonstrates the critical regulation of CD8⁺ T cell memory formation by NAC1, likely through IRF4, and suggests that targeting NAC1 may provide a potentially effective approach to enhancing vaccine efficacy.

### Methods and Materials

Cell lines and mice. C57BL/6 (B6), Rag1^{-/-} and FOXP3-IRES-mRFP (FIR) reporter mice were purchased from The Jackson Laboratory (Bar Harbor, ME). NAC1^{-/-} mice were generated by Dr. Jian-long Wang and crossed in the C57BL/6 background for more 10 generations. B16-F10 (CRL-6475) and A2058 (CRL-11147) cell lines were obtained from ATCC (Manassas, VA). Platinum-E (Plat-E) cell lines were purchased from Cell Biolabs (San Diego, California, USA). B16-F10 cells transfected to express chicken ovalbumin (OVA) (B16-OVA) have been previously described. OT-I TCR Tg mice, B6. Thy1.1 Tg mice and NSG (NOD-scid IL2R^{gnull}) mice, 6-8 weeks, were purchased from The Jackson Laboratory (Bar Harbor, Maine, USA). All the animal experiments were performed in compliance with the regulations of The Texas A&M University Animal Care Committee (IACUC) and in accordance with the guidelines of the Association for the Assessment and Accreditation of Laboratory Animal Care.

T cell culture. T cells were cultured in 48-well plates containing 1 ml RPMI 1640 (Invitrogen) with 10% fetal calf serum (Omega Scientific, CA). T cell isolation kits including mouse CD4⁺ (#130-104-454), CD8a⁺ (#130-104-075) and CD4⁺ CD25⁺ Treg (#130-091-041), T cell activation/expansion kit (#130-093-627) and Treg expansion kit (#130-095-925) were purchased from the Miltenyi Biotec (Auburn, CA). Recombinant mouse TGF-β (#763104), IL-10 (#575106), and TNF-α (#575206) were obtained from BioLegend (San Diego, CA).

Cytokine secretion, cell recovery, and proliferation cell division. IL-2 and IFN-γ were measured using ELISA (Song et al., 2004) and TGF-β1 (#141403, BioLegend) and IL-10 (#130-090-489, Miltenyi Biotec) were measured using flow cytometry. In vitro T cell survival was determined using trypan blue exclusion. Proliferation/division of T cells were measured using the CellTrace™ CFSE Cell Proliferation Kit (#C34554, Invitrogen).

Metabolic assays. Purified CD4⁺ Tregs were plated in the Cell-Tak-coated Seahorse Bioanalyzer XFe96 culture plates (300,000 or 100,000 cells/well, respectively) in assay medium consisting of minimal, unbuffered DMEM supplemented with 1% BSA and 25 mM glucose, 2 mM glutamine (and 1 mM sodium pyruvate for some experiments). Basal rates were taken for 30 min, and then streptavidin-complexed anti-CD3bio at 3 mg/mL±anti-CD28 at 2 mg/mL or PMA (CAS 16561-29-8) (Fisher) was injected and readings were taken for 1-6 hr. In some experiments, oligomycin (2 mM), carbonyl cyanide p-trifluoromethoxyphenylhydrazone (FCCP) (0.5 mM), 2-deoxy-d-glucose (10 mM) and rotenone/antimycin A (0.5 mM) were injected to obtain maximal respiratory and control values. Because ECAR values tend to vary among experiments, both a representative trace and normalized data (calculated as the difference between maximal and basal ECAR values) were shown in the figures.

In vitro mouse Treg generation. Naive CD4⁺CD25⁻ T cells from the LNs and spleen of WT or NAC1^{-/-} mice were incubated with the indicated reagents including TGF-β in the CellXVivo™ Mouse Treg Cell Differentiation Kit (#CDK007, R&D Systems) for 5 days.

In vitro Treg suppression assay. CD4⁺ CD25⁺ Tregs were co-cultured with the CFSE-labeling CD4⁺ CD25⁻ responder T cells from the pooled LNs and spleen of C57BL/6 mice in a ratio of 1:1. To stimulate T cells, the mixed T cells were treated with the T cell activation/expansion kit (#130-093-627; Miltenyi Biotec). As controls, CD4⁺ CD25⁺ Tregs and CD4⁺ CD25⁻ responder T cells were cultured without any stimulus. Suppression of responder T cells was determined by measuring CFSE dilution.

Retroviral transduction. Full-length cDNA of NAC1 was provided by Dr. Ie-Ming Shih and Tian-Li Wang (John Hopkins University (Nakayama et al., 2006), and subcloned into the Mig vector containing GFP for retroviral transduction of mouse Tregs (Haque et al., 2016a).

Antibodies and reagents. PE-, PE/Cy7, Alexa 647, APC or APC/Cy7-conjugated anti-mouse CD4 (GK1.5), CD8 (53-6.7), CD25 (3C7), CD45RB (C363-16A), CD25 (3C7), CD44 (IM7), CD117 (2B8), TCRVβ (H57-597), TGF-β1(TW7-16B4) and FoxP3 (MF-14) were purchased from BioLegend (San Diego, CA). Rabbit NAC1 antibody (#4183) and actin (#8457) were purchased from Cell Signaling (Beverly, MA). Anti-NAC1 antibody (ab29047) for immunoprecipitation was obtained from Abcam (Cambridge, MA). Cycloheximide were purchased from Sigma-Aldrich Corporation (Sigma-Aldrich, St Louis, MI, USA).

RT-PCR. Retrovirally transduced Tregs with Mig or Mig-NAC1 were unsorted or sorted, and total RNA was extracted from the Tregs using QIAgen RNeasy mini kits. Samples were subjected to reverse transcription using a high-capacity cDNA synthesis kit (Applied Biosystems). PCR analysis was performed using TaqMan real-time PCR (Thermo Fisher Scientific). Primers used are: FoxP3 forward: 5′-CCCAGGAAAGACAGCAACCTT-3′, FoxP3 reverse: 5′-TTCTCACAACCAGGCCACTTG-3′; NAC1 forward: 5′-TGC TTA GTT AAC TTA CTG CAG GGC TTC AGC CGA-3′, NAC1 reverse: 5′-TAA GCA CTC GAG ATG GCC CAG ACA CTG CAG ATG-3′.

CpG DNA methylation. CpG DNA methylation was analyzed by bisulphite treatment of RNase-treated genomic DNA, followed by PCR amplification and pyrosequencing (Pyro Q-CpG), which was performed by EpigenDX. Eight mouse genes, including FoxP3, Ctla4, Ikzf2, Ikzf4, Tnfrsf18, Il2ra, Cd274 and Irf4, were screened for methylation percentage in various regulatory regions. Sequences analyses for FoxP3 were: FoxP3 promoter, FoxP3 CNS2 and FoxP3 3′ region.

RNA-Seq. Tregs were mechanically disrupted and homogenized using a Mini-BeadBeater-8 (BioSpec Products, Bartlesville, Oklahoma). RNA was extracted using a RNeasy Mini Kit (Qiagen, Valencia, California). RNA concentration and integrity were measured using an Agilent 2100 Bioanalyzer (Agilent Technologies, Santa Clara, California). All samples had an RNA Integrity Value (RIN) of >7.5. RNA-Seq libraries were prepared using the Illumina TruSeq Stranded mRNA Library Prep Kit (Illumina, San Diego, California) and sequenced on an Illumina HiSeq 2500 Sequencer (Illumina, San Diego, California) as 75 base pair (bp) paired-end reads.

CHIP-seq. ChIP was performed as described (Ubaid et al., 2018), with some modifications. Tregs were subjected to sonication using a Bioruptor® Pico sonication device (Diagenode) to obtain 100-500-bp chromatin fragments. A total of 250 μg of sonicated chromatin fragments were incubated with 10 μg of NAC1 antibody for crosslinking with magnetic beads (no. 11201D, Dynabeads® M280 sheep anti-mouse IgG, Dynal Biotech, Invitrogen). The cross-linked samples were reversed at 65° C. for overnight, and the precipitated DNA was treated with RNase A and proteinase K, and then purified using the QIAquick PCR purification kit (QIAGEN). The DNA libraries were prepared following the guidelines from Illumina (Fasteris Life Sciences; Plan-les-Ouates, Switzerland). Input DNA was sequenced and used as a control. The DNA libraries were sequenced on Illumina HiSeq2500, producing 25-35 million reads per sample.

ATAC-Seq. Tregs were freshly dissected and processed for ATAC-seq. In brief, the tissues were resuspended in 1 ml of lysis buffer (1×PBS, 0.2% NP-40, 5% BSA, 1 mM DTT, protease inhibitors), followed by Dounce homogenization with a loose pestle using 20 strokes. The lysates were then filtered through a 40-μm cell strainer, and the nuclei were collected by centrifugation at 500 g for 5 min. Tagmentation was performed immediately according to the reported ATAC-seq protocol (Yang et al., 2020).

Pulse-chase analysis. Isolated Tregs from WT or NAC1^{-/-} mice were activated and expanded with kits (#130-104-454 and #130-095-925; Miltenyi Biotec), then treated with cycloheximide (150 μg/ml) for various periods of time. FoxP3 protein was analyzed by immunoblotting.

Collagen-induced arthritis. C57BL/6 mice (4 months old) were injected at the base of the tail with 0.1 mL of emulsion containing 100 μg of bovine type II collagen (CII) (Chondrex, Redmond, WA, USA) in complete Freund's adjuvant (CFA) (Chondrex), using a 1-mL glass tuberculin syringe with a 26-gauge needle. Mice were assessed for arthritis in the paws (Haque et al.).

DSS-induced colitis. Colitis was induced in mice by oral ingestion of 3% dextran sulfate sodium (DSS, SKU 02160110-CF; MP Biomedicals) in drinking water for 5 days. The severity of colitis activity was graded on designated dates as described (Wirtz et al., 2017). Body weight, occult or gross rectal bleeding, and feces consistency (on scales of 0-4) were monitored for each mouse. The resultant IBD disease activity index is the average of the scores of the colitis symptoms. The occult blood in mouse fecal samples was detected using Hemoccult Test Kit (Beckman Coulter Inc, Fullerton, CA).

T cell transfer model of colitis. Naive CD4⁺ T effectors (Teffs, CD45RB^{hi}CD25⁻) from B6 mice and CD4⁺ Tregs (CD45RB^{lo}CD25⁺) from WT or NAC1^{-/-} mice were purified using a high-speed cell sorter. Naive CD4⁺ Teffs (6×10⁵ cells/mouse) without or with Tregs (2×10⁵ cells/mouse) were then i.p. transferred into Rag1^{-/-} mice. Body weights were recorded twice a week. When loss of body weight exceeded 20% after transfer, the host mice were sacrificed.

Histology and immunohistochemistry. Joint or colon tissues were fixed with 10% neutral formalin solution (VWR, West Chester, PA), and the fixed samples were prepared and stained with H&E as described in Lei et al., 2011. For immunofluorescent microscopy, the tissues were frozen in cryovials on dry ice immediately following resection. Cryo-sectioning and immunofluorescent staining were performed as described in Lei et al., 2011.

Statistical analysis. Multiple Paired or unpaired Student's t-test or one-way analysis of variance, simple linear regression and survival curve comparison were performed to analyze the differences between the groups, using GraphPad Prism (GraphPad Software, San Diego, CA); significance was set at 5%.

Reagents. H-2K^{b} VACV B8R (TSYKFESV) Tetramer (#TB-M538-1, MBL), anti-mouse CD36 antibody (clone HM36, BioLegend), anti-human/mouse/rat NAC1 antibody (clone SWN-3, BioLegend), anti-human FoxP3 antibody (clone 206D, BioLegend), and L-(⁺)-Lactic acid (#ICN19022805, MP Biomedicals)

Viral infection. VACV infection was performed by an intraperitoneal injection of viruses (2×10⁶ PFU/mouse) as described in Salek-Ardakani et al., 2008.

Murine Melanoma Model. WT or NAC1^{-/-} Tregs (1×105) were injected s.c. in the flank region of the recipient mice inoculated with 1×10⁶ B16 tumor cells. Tumor sizes were measured by a caliper and tumor volumes were calculated as: V=long diameter×short diameter²×0.52.

Cell Culture. A2058, and Plat-E cell lines were cultivated in DMEM medium supplemented with 10% heat-inactivated fetal calf serum, 0.5% penicillin/streptomycin. B16-OVA were cultivated in RPMI 1640 medium supplemented with 10% heat-inactivated fetal calf serum, 0.5% penicillin/streptomycin. All reagents were from Sigma-Aldrich (St Louis, MI).

PBMC isolation. PBMCs were isolated from healthy donor blood samples from the Gulf Coast Regional Blood Center (Houston, Texas, USA). Mononuclear cells were isolated by the density gradient centrifugation using Ficoll-Paque PLUS from Sigma-Aldrich (GE17-1440-02, St Louis, Michigan, USA).

CD8⁺ T cell purification, expansion, and transduction. Murine CD8⁺ T cells were isolated from the pooled lymph nodes and spleen of OT-I TCR Tg mice by the magnetic bead separation using the MojoSort Mouse CD8 T Cell Isolation Kit from BioLegend (#480008, San Diego, California, USA). Purified CD8⁺ T cells were activated with anti-CD3 (5 μg/mL, plate-coated) and anti-CD28 (5 μg/mL, soluble) and cultured in RPMI containing 10% FBS, 100 μg/mL penicillin/streptomycin, 2 mM L-glutamine, 20 mg/mL NEAA, and 5 μl/mL β-mercaptoethanol from Sigma-Aldrich (#M6250, St Louis, Michigan, USA). Human CD8⁺ T cells were isolated from PBMCs by magnetic bead separation using the MojoSort human CD8 T Cell Isolation Kit from BioLegend (#480012, San Diego, California, USA). Purified CD8⁺ T cells were activated and expanded with anti-CD3 (5 μg/mL, plate-coated) and anti-CD28 (5 μg/mL, soluble), human rIL-2 (100 U/mL) and cultured in RPMI containing 10% FBS, 100 μg/mL penicillin/streptomycin, 2 mM L-glutamine, 20 mg/mL NEAA, and 5 μl/mL p-mercaptoethanol from Sigma-Aldrich (St Louis, Michigan, USA). Two days after activation, human CD8⁺ T cells were transduced with anti-tyrosinase TCR construct. The retro-viral plasmid pMSGV1 backbone integrated with anti-tyrosinase TCR was a gift from Dr. Richard Morgan as previously described. The construct was transfected into the packing cell line Plat-E. After 48 hours, the retrovirus-enriched supernatant was harvested and purified with 0.45 μM filter from Sigma-Aldrich (#SLHV004SL, St Louis, Michigan, USA). Retrovirus was enriched, and CD8⁺ T cells were transduced with a RetroNectin (#T202)-coated plate according to the manufacturer's instructions from Takara Bio (San Jose, California, USA)

Plasmid transfection and retroviral transduction. B16-OVA mouse melanoma cells were transfected with CRISPR plasmids (BTBD14B CRISPR/Cas9 KO Plasmid (m) from Santa Cruz Biotech (#sc-426213, Dallas, Texas, USA) to specifically knockout the expression of NACC1. Transfected GFP⁺ cells were sorted using a high-speed cell sorter. In parallel, we used B16-OVA cell line as a non-transfected control (WT). In the human melanoma cell line A2058, NACC1 was knocked out with CRISPR plasmids (BTBD14B CRISPR/Cas9 KO Plasmid (h)) from Santa Cruz Biotech (#sc-410250, Dallas, Texas, USA). Transfected GFP⁺ cells were sorted using a high-speed cell sorter. Non-transfected cells (WT) served as control. cDNA of LDHA was obtained from Dr. Sunmin Kang (Emory University, Georgia, USA). We subcloned the cDNA of LDHA into the retroviral vector backbone pMIG (#9044, Addgene) as previously described. Cloning was confirmed by PCR amplification and gene sequencing. The pMIG-LDHA plasmid was transfected into the packaging cell line of Plat-E. After 48 hours, the retrovirus-enriched supernatant was harvested and purified with 0.45 μM filter from Sigma-Aldrich (St Louis, MI). NAC1 KO B16-OVA and NAC1 KO A2058 tumor cells were cultured with this viral supernatant overnight with 10 μg/mL polybrene from Sigma-Aldrich (St Louis, MI). The transduced GFP⁺ cells were sorted using a high-speed cell sorter. In parallel, we used the pMIG transduced NAC1 KO B16-OVA and NAC1 KO A2058 tumor cells as mock controls.

In vitro analysis of CD8⁺ T cells. For cytotoxicity analysis, after 24 hours of activation 5×10⁵ CD8⁺ T cells were cultured with WT B16-OVA and NAC1 KO B16-OVA tumor cells in the ratio of 1:5 and 1:10 respectively. Cytotoxicity was measured by the CytoTox 96 Non-Radioactive Cytotoxicity Assay from Promega (#G1780, Madison, Wisconsin, USA). For cytokine expression analysis, the CD8⁺ T cells were cultured with the conditional medium (CM) that was collected from WT B16-OVA and NAC1 KO B16-OVA melanoma cells for 12 hours. Before harvesting the cells, the cells were blocked with the Monensin Solution from BioLegend (#420701, San Diego, California, USA) for 4 hours. For apoptosis analysis, after 12 hours of activation the CD8⁺ T cells were incubated with the CM that was collected from WT B16-OVA and NAC1 KO B16-OVA cells for 12 hours, in the absence or presence of L-LA from Sigma-Aldrich (St Louis, MI). The apoptosis analysis was performed using APC Annexin V Apoptosis Detection Kit (#640920) with the Aqua Live/Dead (#423101) from BioLegend (San Diego, California, USA).

Glycolysis analysis. For lactate production analysis, the LA concentration in the supernatants of cells cultured for 24 hours was measured enzymatically using a Lactate-Glo Assay from Promega (#J5021, Madison, Wisconsin, USA). For glucose-uptake production analysis, the cells were cultured for 24 hours with an associated fresh medium before harvesting these cells. Intracellular glucose concentration was quantified enzymatically using a Glucose Uptake-Glo Assay from Promega (#J1341, Madison, Wisconsin, USA, USA). For the Seahorse glycolysis analyzer: 10 000 cells/well were seeded in a Seahorse XF96 cell culture plate and were allowed to adhere overnight. The next day, plates were further incubated at 37° C. in a non-C02 incubator for 30 min, followed by testing with Glycolytic Rate Assay Kit of Agilent Seahorse XF (#103344-100). One hour before measurement, cell culture media was replaced with Seahorse XF DMEM with pH 7.4, containing 1 mM pyruvate, 2 mM glutamine, and 10 mM glucose. The following concentrations of each drug were used for extracellular acidification rate (ECAR) acquisitions: 2-DG 50 μM, Rote-none and Antimycin A, 0.5 μM. All reagents were from Agilent (Santa Clara, California, USA).

Quantitative real-time PCR. Total RNA of WT B16-OVA, NAC1 KO B16-OVA, WT A2058 and NAC1 KO A2058 tumor cells was obtained using the RNeasy Mini Kits from QIAGEN (#74104, Germantown, Maryland, USA). Complementary DNA was synthesized with a Maxima H Minus First Strand cDNA Synthesis Kit and amplified by qPCR with PowerUp SYBR Green Master Mix (#A25742, Thermo Scientific, Massachusetts, USA) using the CFX96 Touch Real-Time PCR Detection System from Bio-Rad (Hercules, California, USA). The primer sequences are included in online supplemental table 1.

Flow Cytometric analysis. For mouse tissue, each tumor was minced using the mouse Tumor Dissociation Kit from Miltenyi Biotec (#130-096-730, Auburn, California, USA). All samples were then washed with flow cytometry buffer, and the cells were further passed through a 100 μm cell strainer. The samples were incubated for 30 min at 4° C. in the dark with the following antibodies: APC-PD-1 (#135210), BV711-TIM3 (#134021), PE-IL-2 (#503808), FITC-IFN-γ (#505806), APC-Thy1.2 (#140331), Pacific Blue-Granzyme B (#515408), PE-TNFα (#506306), PE-CD4 (#100408), APC-perforin (#154304), APC-Annexin V (#640941) and FITC-CD8 (#100706) from BioLegend (San Diego, California, USA). Intracellular staining was performed after incubation of single-cell suspensions with BD GolgiStop from BD Biosciences (#AB_2869012, San Diego, California, USA) in medium for 4 hours using Intracellular Staining Permeabilization Wash Buffer and Fixation Buffer from BioLegend (#421002, San Diego, California, USA). Stained cell populations were acquired by LSRFortessa from BD Biosciences (Franklin, New Jersey, USA), and the results were analyzed by using FlowJo software from Tree Star (Ashland, Oregon, USA).

Immunoblotting. Cells were lysed in ice-cold RIPA Lysis Buffer (#89900) for 30 min. Pierce protease inhibitors and Halt phosphatase inhibitors from Thermo Fisher Scientific (Waltham, Massachusetts, USA). Protein concentration was measured using the Bio-Rad protein assay kit (#5000002, Hercules, California, USA). Equal protein concentrations were loaded per condition. Proteins were separated with Nu-PAGE 4-12% Bis-Tris gels (#NP0321BOX) using MES×1 running buffer (#NP0002) at 150V constant. Protein was transferred using the wet transfer Xcell II Blot Module. Membranes were incubated in blocking buffer (5% milk in TBST) for 1 hour before incubation with primary antibodies at 4° C. overnight. After washing 3 times for 10 min with TBST, membranes were incubated with HRP-conjugated antibodies for 1 hour at room temperature. Primary antibodies used were NAC1 (#ab29047, Abcam), β-actin (#664802, BioLegend), and LDHA (#AF7304, R&D system). Other reagents were from Thermo Fisher Scientific (Waltham, Massachusetts, USA).

Murine melanoma models. For the B16 melanoma model, 1×10⁶B16-OVA or NAC1 KO B16-OVA melanoma cells were s.c. inoculated into the right flank of B6. Thy1.1 mice (N=5). In vitro activated OT-I CD8⁺ T cells (5×10⁶) were washed and re-suspended in cold PBS before i.v. injection into B6. Thy1.1 mice through the tail vein when the tumor sizes reached around 50 mm³. Tumor sizes were calculated as V=long diameter×(short diameter/2). When mice-bearing tumors reach a maximum size of 2000 mm³, tumors were prepared for analysis. In the mouse model of human melanoma, 1×10⁶ human WT A2058 or NAC1 KO A2058 melanoma cells were s.c. inoculated into the right flank of NSG mice. Human CD8⁺ cells were activated and transduced with an anti-tyrosinase TCR, and the transduced GFP⁺ cells were resuspended in cold PBS before intravenous injection in NSG mouse (5×10⁶/mouse) through the tail vein when the tumor sizes reached around 50 mm3.

Histology and immunohistochemistry. Tumor tissues were fixed with 10% neutral formalin solution (VWR, West Chester, Pennsylvania, USA), and the fixed samples were prepared and stained with H&E as described. The tissues were frozen in cryovials on dry ice immediately following resection for immunofluorescent microscopy. Cryosectioning and immunofluorescent staining were performed as described. FITC-CD8 and Alexa Fluor 647 CD90.2 from BioLegend (San Diego, California, USA) were used to detect the tumor-infiltrating OT-I CD8⁺ T cells.

Database analyses. Kaplan-Meier estimation curves for overall survival (OS) of individual metastatic melanoma patients were generated with the microarray analysis and visualization platform R2 (http://r2.amc.nl) by using the ‘R2: Tumor Skin Cutaneous Melanoma The Cancer Genome Atlas (TCGA)-470-rsem-tcgars’. Correlation of LDHA expression with NACC1 was determined with the ‘R2: Tumor Skin Cutaneous Melanoma-TCGA-470-rsem-tcgars’ dataset containing data from 470 melanoma patients (http://cancergenome.nih.gov). Pearson's correlation was calculated with the transform 2 log setting. The NAC1 expression of skin cancers was determined with The Cancer Cell Line Encyclopedia (https://depmap.Org/portal/ccle/), including 70 types of human skin cancer cell line. The infiltration level of CD8⁺ T cells in SKCM was determined with Tumor Immune Estimation Resource (TIMER2.0) with official instruction.

T Cell Isolation, T Cell Activation, and T Cell Culture. CD8⁺ T cells were isolated from mouse spleen and lymph nodes (LNs) using MojoSort Mouse CD8⁺ Naive T Cell Isolation Kit. T cells were activated by plate-coated 4 μg/mL anti-CD3 (clone 2C11) and 4 μg/mL anti-CD28 (clone 37.51) antibodies (Abs) in T cell culture medium. T cells were cultured in RPMI medium with 10% FBS, 1% NEAA, 55 μM 2-ME, 2 mM L-glutamine, and 1% Penicillin-streptomycin. T cells were split based on their density.

Virus Preparation and Titration. Vaccinia virus Western Reserve strain (VACV-WR) stock was prepared and grown in HeLa cells. When the HeLa cells neared confluency, they were infected at the optimal multiplicity of infection (MOI) around 2 PFU/cell. Later, the vaccinia virus stock was titrated with Vero C1008 cells through a plaque assay. When the Vero cells reached confluence, the virus stock was serial diluted and added to each 6-well. After two days of incubation, the plaque numbers were counted after Crystal Violet Staining. The detailed methods for both are in the protocol described previously. The viral stock was then kept at −80° C. for future usage.

Viral Infection. VACV-WR infection was performed by intraperitoneal injection (2×106 PFU/mouse) as described previously.

Western Blot. T cell protein was extracted with M-PER™ Mammalian Protein Extraction Reagent (Thermo Scientific #78503). The protein samples were then collected and quantified using BCA protein assay (Thermo Fisher #23225). The IRF4 primary Ab used is rabbit anti-mouse IRF4 Ab (CST #62834T). The j-ACTIN primary Ab utilized is rat anti-mouse ACTIN Ab (BioLegend #664802). The second HRP-conjugated anti-rabbit (BioLegend #406401) and anti-rat (BioLegend #405405) Abs were purchased from BioLegend.

Memory T Cell Tetramer Staining and Flow Cytometry. Pooled superficial cervical, axillary, brachial, and inguinal lymph nodes were combined with the spleen of each mouse for analysis. The tissues were pulverized, and cells were filtered using a 40 μm cell strainer. T cell preparation and staining with different surface markers were described in a previous publication. Following this, the VACV tetramer was used to detect VACV-specific T cells at room temperature for 30 min in a cell-staining buffer (BioLegend #420201). The MHC class I B8R (TSYKFESV) tetramer was synthesized in the NIH Tetramer Core. All flow cytometry experiments were completed in the Texas A&M University COM-CAF core facility with the BD Fortessa X-20. The final plotting was performed in FlowJo Software.

RNA Extraction, cDNA Synthesis, and qPCR. NA extraction was completed with the RNeasy Mini Kit (Qiagen #74104). DNA was removed by TURBO DNA-free Kit (Ambion #AM1907). The cDNA was synthesized with High-capacity cDNA Reverse Transcription Kit (Thermo Fisher #4368813). The qPCR was accomplished with primers described below. Irf4: Forward-GCAATGGGAAACTCC-GACAGT, Reverse-CAGCGTCCTCCTCACGATTGT. Gapdh: Forward-GTTGTCTCCTGCGACTTCA, Reverse-GGTGGTCCAGGGTTTCTTA. Bio-Rad CFX384 Touch Real-Time PCR Detection System was used to perform qPCR.

Seahorse Assay. The Seahorse assays were performed with Agilent Seahorse XF Cell Mito Stress Kit (#103010-100) and Agilent Seahorse XF Glycolytic Rate Assay Kit (#103346-100) according to their user guides. Approximately 2×10⁵ cells were plated in each well of the microplate before the glycolytic rate and mitochondrial stress tests. The drugs were injected into each sample at different times. The Extra Cellular Acidification Rate (ECAR) was measured in the glycolytic rate and the Oxygen Consumption Rate (OCR) was tested to indicate oxidative phosphorylation.

CHIP-Seq. The CHIP-seq sample preparation was finished with Zymo-Spin CHIP Kit (#D5209). The Ab utilized for NAC1 was the mouse NAC1 Ab (BioLegend #849301). The IgG control Ab selected was Go-ChIP-Grade™ Purified Mouse IgG1 (BioLegend #401409). The sample was sequenced in TIGSS Molecular Genomics Core at Texas A&M University. The sequencing data were then visualized by the Integrative Genomics Viewer (IGV).

CFSE Labeling. CD8⁺ T cells were isolated from pooled LNs and spleen. Then, the T cells were labeled with CFSE for 10 min at room temperature. Then, cells were activated with precoated anti-CD3 and soluble anti-CD28 Abs as we described in Section 2.2. After two days, the samples were analyzed by flow cytometry.

When ranges are disclosed herein, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited. Other objects, features and advantages of the disclosure will become apparent from the foregoing drawings, detailed description, and examples. These drawings, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein. It should be understood that although the disclosure contains certain aspects, embodiments, and optional features, modification, improvement, or variation of such aspects, embodiments, and optional features can be resorted to by those skilled in the art, and that such modification, improvement, or variation is considered to be within the scope of this disclosure.

What is claimed is:

1. A method for enhancing or inducing an anti-tumor response in a patient, the method comprising:
administering to the patient a therapeutically effective amount of an inhibitor of expression or activity of nucleus accumbens-associated protein-1 (NAC1).

2. The method of claim 1, wherein the anti-tumor response is an increase in CD8⁺ T cell-mediated anti-tumor immunity.

3. The method of claim 1, wherein the anti-tumor response is a persistent anti-tumor T cell memory.

4. The method of claim 1, wherein the patient has been administered an adoptive cell transfer therapy.

5. The method of claim 4, wherein the adoptive cell transfer therapy is a chimeric antigen receptor T-cell therapy.

6. The method of claim 4, wherein the adoptive cell transfer therapy is a tumor-infiltrating lymphocyte therapy.

7. The method of claim 1, wherein the inhibitor of NAC1 is a NAC1-targeted siRNA.

8. The method of claim 7, wherein the NAC1-targeted siRNA is administered as a nanoliposome.

9. The method of claim 1, wherein the inhibitor of NAC1 is a CRISPR/Cas-based genome editing composition comprising one or more vectors encoding: (a) one or more guide RNAAs (gRNAs) that are complementary to one or more target sequences in a NAC1 gene and (b) a nucleic acid sequence encoding a Clustered Regularly Interspaced Short Palindromic Repeat (CRISPR)-associated endonuclease, whereby the one or more gRNAs hybridize to the NAC1 gene and the CRISPR-associated endonuclease cleaves the NAC1 gene, and wherein the NAC1 gene is deleted in the patient relative to a patient to whom the CRISPR/Cas-based genome editing composition is not administered.

10. The method of claim 1, wherein the inhibitor of NAC1 corresponds to Formula I:

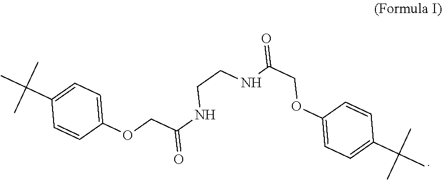 [SMILES: CC(C)(C)c1ccc(OCC(=O)NCCNC(=O)COc2ccc(C(C)(C)C)cc2)cc1]

11. The method of claim 1, wherein the inhibitor of NAC1 is an isolated antibody or its binding fragment thereof that binds to a NAC1 protein.

12. A method of treating an autoimmune disorder in a patient, the method comprising:
administering a therapeutically effective amount of an inhibitor of nucleus accumbens-associated protein-1 (NAC1).

13. The method of claim 12, wherein the inhibitor of NAC1 corresponds to Formula I:

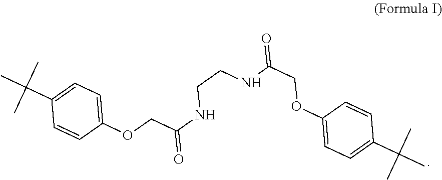 [SMILES: CC(C)(C)c1ccc(OCC(=O)NCCNC(=O)COc2ccc(C(C)(C)C)cc2)cc1]

14. The method of claim 12, wherein the inhibitor of NAC1 is a NAC1-targeted siRNA administered as a nanoliposome.

15. The method of claim 12, wherein the inhibitor of NAC1 is a CRISPR/Cas-based genome editing composition comprising one or more vectors encoding: (a) one or more guide RNAs (gRNAs) that are complementary to one or more target sequences in a NAC1 gene and (b) a nucleic acid sequence encoding a Clustered Regularly Interspaced Short Palindromic Repeat (CRISPR)-associated endonuclease, whereby the one or more gRNAs hybridize to the NAC1 gene and the CRISPR-associated endonuclease cleaves the NAC1 gene, and wherein the NAC1 gene is deleted in the patient relative to a patient to whom the CRISPR/Cas-based genome editing composition is not administered.

16. The method of claim 12, wherein the autoimmune disorder is autoimmune arthritis.

17. The method of claim 12, wherein the autoimmune disorder is autoimmune colitis.

18. A method of enhancing effectiveness of a vaccine in a subject, the method comprising:
administering to the subject a therapeutically effective amount of an inhibitor of nucleus accumbens-associated protein-1 (NAC1).

19. The method of claim 18, wherein the inhibitor of NAC1 is administered before, after or concurrent with the vaccine.

20. The method of claim 18, wherein the vaccine is a COVID-19 vaccine.


SEQUENCE LISTING (11 sequences)

SEQ ID NO: 1 - RNA, 27 nt, synthetic construct
  source 1..27: mol_type = other RNA
tgatgtacacgttggtgcctgtcacca

SEQ ID NO: 2 - RNA, 25 nt, synthetic construct
  source 1..25: mol_type = other RNA
tgtagcagaagctgaggatctgctg

SEQ ID NO: 3 - DNA, 21 bp, synthetic construct
  source 1..21: mol_type = other DNA
cccaggaaagacagcaacctt

SEQ ID NO: 4 - DNA, 21 bp, synthetic construct
  source 1..21: mol_type = other DNA
ttctcacaaccaggccacttg

SEQ ID NO: 5 - DNA, 33 bp, synthetic construct
  source 1..33: mol_type = other DNA
tgcttagttaacttactgcagggcttcagccga

SEQ ID NO: 6 - DNA, 33 bp, synthetic construct
  source 1..33: mol_type = other DNA
taagcactcgagatggcccagacactgcagatg

SEQ ID NO: 7 - protein, 8 aa, synthetic construct
TSYKFESV

SEQ ID NO: 8 - DNA, 21 bp, synthetic construct
  source 1..21: mol_type = other DNA
gcaatgggaaactccgacagt

SEQ ID NO: 9 - DNA, 21 bp, synthetic construct
  source 1..21: mol_type = other DNA
cagcgtcctcctcacgattgt

SEQ ID NO: 10 - DNA, 19 bp, synthetic construct
  source 1..19: mol_type = other DNA
gttgtctcctgcgacttca

SEQ ID NO: 11 - DNA, 19 bp, synthetic construct
  source 1..19: mol_type = other DNA
ggtggtccagggtttctta